(19) United States
(12) Patent Application Publication
(10) Pub. No.: US 2024/0382185 A1
(43) Pub. Date: Nov. 21, 2024

(54) Environmentally Responsible Diagnostic Test System with Advanced Collection, Assay, and Data Management Innovations

(71) Applicant: ASSAYA LLC, Atlanta, GA (US)

(72) Inventor: Clas Sivertsen, Lilburn, GA (US)

(21) Appl. No.: 18/197,683

(22) Filed: May 15, 2023

(51) Int. Cl. A61B 10/02; A61B 10/00; A61L 2/08; A61L 2/20

(52) U.S. Cl. CPC A61B 10/02; A61B 10/0051; A61L 2/081; A61L 2/206

(57) ABSTRACT

A standing packet, known as a “spacket”, is disclosed. The spacket includes a sheeted material having a first sheet and a second sheet sealed to form a tube. The first sheet and second sheet have perforations along at least a portion of the top part of the formed tube. A third sheet is placed between the first sheet and the second sheet and adhered to the formed tube. The third sheet forms a concave internal cup folded in such a way that the tube stands upright during use, whereby the top part of the formed tube may be removed during use.

### BACKGROUND OF THE INVENTION

The field of diagnostic testing has seen significant advancements over the years, leading to improved detection and management of various diseases and health conditions. However, despite these advancements, there are still several challenges and issues that need to be addressed in order to further enhance the efficiency, sustainability, and user experience of diagnostic testing processes.

One of the primary concerns is the environmental impact of diagnostic tests and their components, which often generate a considerable amount of plastic waste. 2 Billion lateral flow assays are produced every year—all of them go into landfills and waste. It takes approximately 400 years to degrade. This waste not only contributes to pollution and resource depletion but also increases the overall cost of healthcare. Therefore, there is a pressing need for the development of more environmentally friendly alternatives to traditional diagnostic test components, such as specimen collection devices, reagent containers, and packaging materials.

Another challenge faced by healthcare professionals and end-users is the complexity and inefficiency of diagnostic test procedures. This complexity often leads to errors in sample collection, handling, and result interpretation, which can have significant consequences for patient care. Streamlining the diagnostic testing process and simplifying the user experience are essential for reducing the risk of errors and improving overall testing efficiency.

Furthermore, the lack of standardization in diagnostic test components and packaging often results in increased costs, inefficient use of storage and shipping space, and potential confusion among healthcare professionals and end-users. The development of standardized, user-friendly packaging systems and components is necessary to overcome these issues and facilitate easier handling and transportation of medical products.

In addition, there is a growing demand for non-invasive diagnostic testing methods, which can provide rapid and accurate results without causing discomfort or harm to patients. The development of innovative, non-invasive testing techniques that can detect pathogens and other health indicators in a quick and efficient manner is crucial for improving patient care and reducing the need for invasive sampling procedures.

With the increasing reliance on digital technology in healthcare, there is also a need for better data management systems and electronic resources that can streamline the diagnostic testing process, reduce paper waste, and enhance user experience. Integrating digital platforms and tools into diagnostic testing procedures can provide healthcare professionals and end-users with more efficient, accessible, and user-friendly solutions.

Innovations in this area should focus on improving sustainability, streamlining processes, enhancing user experience, promoting standardization, and developing non-invasive testing methods, all while leveraging the potential of digital technology to revolutionize the traditional testing process.

### SUMMARY OF THE INVENTION

The present invention relates to a comprehensive diagnostic test system designed to address various issues associated with the current state of medical testing, including sample collection, test execution, result interpretation, data management, and environmental impact. This system comprises a suite of eleven distinct but interconnected innovations, each addressing specific challenges and inefficiencies within the realm of medical testing. Collectively, these inventions form an advanced, environmentally responsible diagnostic test system that substantially improves upon the current state of the art.

### 1. Sterile Wood Abrasive Collector (SWAC)

The first invention in the system, the Sterile Wood Abrasive Collector (SWAC), addresses the environmental concerns and resource limitations associated with traditional nasal and nasopharyngeal swabs, which are predominantly made from non-renewable materials such as plastics and non-sustainable cotton. The SWAC offers an eco-friendly alternative, as it is constructed from a renewable and biodegradable material-wood.

Furthermore, the SWAC is designed to provide a more comfortable and less invasive experience for patients during sample collection. Current nasal and nasopharyngeal swabs can cause significant discomfort and even injury when inserted into the nose or throat, whereas the SWAC's design reduces the risk of injury and increases patient comfort. The SWAC also allows for a more efficient and effective sample collection, as its design ensures a consistent and reliable sample size, reducing the likelihood of inadequate or contaminated samples.

### 2. Pathogen Exhalate Collector (PEC)

The second invention, the Pathogen Exhalate Collector (PEC), focuses on improving the process of capturing exhaled pathogens during diagnostic testing. Traditional methods for collecting exhaled samples, such as breath condensate collection or cough sampling, can be cumbersome, time-consuming, and prone to contamination. The PEC offers a more efficient and reliable means of collecting exhaled samples by redesigning the collection device itself.

The PEC features an innovative design that allows for optimal airflow and particle capture, while minimizing the risk of contamination or sample loss. This new design ensures that the exhaled sample is representative of the patient's true pathogen load, increasing the accuracy and reliability of diagnostic testing. The PEC also offers the potential for improved patient compliance, as it requires less effort and discomfort during sample collection compared to traditional methods.

### 3. Environmentally Friendly Buffer Test Tube Replacement—“the Spacket”

The third invention, an environmentally friendly buffer test tube replacement, addresses the issues of waste and resource consumption associated with traditional plastic buffer test tubes. Current test tubes are predominantly made from single-use plastics, which are not easily recyclable and contribute to the growing problem of plastic pollution. The proposed replacement is a sustainable, foldable packet known as a “spacket,” which can be used in place of traditional plastic buffer test tubes.

The spacket is designed to be easily opened, filled, and sealed, providing a user-friendly and efficient solution for containing and transporting liquid samples. Additionally, the spacket is constructed from environmentally friendly materials, such as biodegradable or recyclable paper, significantly reducing its environmental impact compared to traditional plastic test tubes.

### 4. Low Environmental Impact Quantitative Assay (LEIQA)

The fourth invention, the Low Environmental Impact Quantitative Assay (LEIQA), offers a more sustainable and versatile alternative to traditional lateral flow assays (LFAs). LFAs are widely used in diagnostic testing due to their simplicity, rapid results, and affordability. However, they can be limited in terms of their detection capabilities, often only able to identify a single analyte per test. The LEIQA is designed to overcome these limitations by providing the ability to detect multiple analytes simultaneously, as well as offering a reduced environmental impact.

The LEIQA features a redesigned test strip that can be customized for detecting a multitude of antigens and antibodies, as well as other biological markers. This multi-analyte detection capability not only improves the efficiency of diagnostic testing but also has the potential to reduce the overall cost and resource consumption associated with conducting multiple tests. Moreover, the LEIQA is designed with sustainability in mind, using eco-friendly materials and manufacturing processes that minimize its environmental footprint.

5. LEIQA Data Management System with Integrated Electronic Instructions for Use (eIFU)

The fifth invention in this suite of innovations addresses challenges and inefficiencies in the management, storage, and access to data related to diagnostic tests and their accompanying instructions for use. This invention is the “LEIQA Data Management System with Integrated Electronic Instructions for Use (eIFU).”

In the realm of diagnostic testing, the proper management and storage of test data, as well as easy access to instructions for use, are crucial for ensuring accurate test results, efficient workflows, and compliance with regulatory requirements. Traditionally, instructions for use have been provided in the form of printed paper manuals or inserts, which can be easily misplaced or damaged. Additionally, these paper-based instructions contribute to environmental waste and can be challenging to update or modify in response to changes in regulatory guidance, product specifications, or scientific knowledge.

To address these issues, the LEIQA Data Management System with Integrated eIFU offers an innovative solution that combines both data management and electronic access to instructions for use. This integrated system allows for efficient storage, retrieval, and management of test data, including test results, lot numbers, and expiration dates, as well as user-specific information such as demographic data, symptoms, and risk factors. By centralizing this information in a digital format, the LEIQA Data Management System simplifies the process of managing and accessing test data, enabling more efficient workflows and improved compliance with regulatory requirements.

In addition to its data management capabilities, the LEIQA Data Management System with Integrated eIFU also provides users with electronic access to instructions for use, replacing the need for printed paper manuals or inserts. The electronic Instructions for Use (eIFU) are accessible through a QR code printed on the test packaging, which can be scanned using a smartphone, tablet, or other internet-enabled devices. Upon scanning the QR code, users are directed to a webpage or app containing the most up-to-date version of the instructions for use. This digital format allows for more dynamic, interactive content, including videos, images, and interactive guides, which can enhance the user's understanding of the test procedure and improve the overall user experience.

By providing electronic access to instructions for use, the LEIQA Data Management System with Integrated eIFU not only eliminates the need for printed paper manuals, reducing environmental waste but also enables more efficient updates and modifications to the instructions in response to changes in regulatory guidance, product specifications, or scientific knowledge. This innovative approach to data management and electronic instructions for use represents a significant advancement in the field of diagnostic testing, offering improved efficiency, accuracy, and sustainability compared to traditional methods.

### 6. Sterile Blood Extractor (SBEx)

An environmentally friendly alternative to traditional lancets and capillary tubes for blood collection, as shown in FIG. 9a-9h

The sixth invention in this suite of innovations focuses on providing an environmentally friendly and efficient alternative to traditional methods of blood collection, specifically lancets and capillary tubes, which are commonly used for diagnostic testing. The invention is the “Sterile Blood Extractor (SBEx).”

Lancets and capillary tubes have been widely used for blood collection in various diagnostic tests. However, these conventional methods have several drawbacks and limitations. One significant issue is the environmental waste generated by the single-use nature of lancets and capillary tubes. These disposable items contribute to the growing problem of medical waste, which can have detrimental effects on the environment and public health. Additionally, traditional blood collection methods can be cumbersome and time-consuming for healthcare professionals and patients alike, as they often require multiple steps and specialized equipment, leading to potential inefficiencies in the diagnostic testing process.

The Sterile Blood Extractor (SBEx) aims to address these concerns by offering an environmentally friendly and efficient alternative to lancets and capillary tubes for blood collection. The SBEx is designed as a reusable device that can efficiently collect blood samples while minimizing environmental waste. The device combines the functions of a lancet and a capillary tube into a single, integrated unit, streamlining the blood collection process and reducing the need for multiple disposable components.

The SBEx features a non-retractable fixed size and position needle, which can be safely and hygienically stored within the device until the needle is used, minimizing the risk of needlestick injuries and contamination. By combining the functions of lancets and capillary tubes into a single, one time usable device, the SBEx significantly decreases the environmental impact of blood collection while enhancing efficiency and safety for healthcare professionals and patients.

In addition to its environmental benefits, the SBEx offers improved functionality and user experience compared to traditional blood collection methods. The device is designed to be easy to use, with ergonomic features that ensure comfortable and accurate blood collection. Moreover, the integrated nature of the SBEx simplifies the blood collection process, reducing the potential for errors and enhancing the overall efficiency of diagnostic testing.

The Sterile Blood Extractor (SBEx) represents a significant advancement in the field of blood collection, offering an environmentally friendly and efficient alternative to traditional lancets and capillary tubes. By addressing the limitations and drawbacks of conventional methods, the SBEx has the potential to improve the diagnostic testing process and contribute to a more sustainable and efficient healthcare system.

### 7. LEIQAPACK—Environmentally Friendly Packaging

The seventh invention, LEIQAPACK, focuses on improving the packaging for medical consumables, such as lateral flow assays (LFAs) and LEIQA kits. The lack of standardization in packaging for medical consumables can lead to increased costs, inefficient storage and shipping, difficulty in handling, and confusion among healthcare professionals and end-users. LEIQAPACK aims to address these issues by utilizing the standardized dimensions of a cigarette carton, which offers several advantages, including streamlined manufacturing, reduced packaging waste, improved storage and transportation efficiency, and ease of handling.

Additionally, LEIQAPACK incorporates innovative design features, such as paper “ears” or “flaps,” which can be repurposed as holders for spackets, small packets containing reagents or other consumables essential for the lateral flow assay or other medical tests. This integration of spacket holders into the packaging itself eliminates the need for additional components, reducing material waste and improving shipping efficiency.

### 8. Specimen Collection Type Color Coding and Iconography.

As the healthcare industry continues to grow and evolve, an increasing number of diagnostic tests are becoming available for various medical conditions, ranging from infectious diseases to genetic disorders. These tests often require different specimen collection methods, such as blood, saliva, urine, or swab samples. Healthcare professionals and end-users must accurately collect and process these specimens to ensure reliable and accurate test results. However, the multitude of diagnostic tests and specimen collection methods can lead to confusion, increasing the risk of errors and potentially compromising patient care.

Current packaging and labeling practices for diagnostic test kits can be inconsistent and unclear, making it difficult for healthcare professionals and end-users to quickly and easily identify the correct specimen collection method for a particular test. This lack of standardization and clarity can result in wasted time, resources, and even misdiagnoses, as incorrect specimen collection may lead to inaccurate test results.

This eighth invention in this suite of innovations addresses these concerns by proposing a color coding scheme, along with accompanying text labels and icons, to clearly and consistently indicate the specimen collection method for various diagnostic test kits. This system, called “SPECIMEN COLLECTION TYPE COLOR CODING AND ICONOGRAPHY,” aims to simplify the identification and selection of appropriate specimen collection methods for different tests, reducing the potential for errors and enhancing overall efficiency in the diagnostic testing process.

The color coding system assigns a specific color, icon, and text label to each type of specimen collection method, allowing healthcare professionals and end-users to easily identify the correct method for a given test at a glance. This visual communication strategy not only reduces confusion but also helps save time by facilitating quick and accurate specimen collection.

In addition to the color coding scheme, the invention incorporates easily recognizable icons and text labels that further reinforce the clarity and consistency of the system. By combining colors, icons, and text labels, the SPECIMEN COLLECTION TYPE COLOR CODING AND ICONOGRAPHY system provides a comprehensive and user-friendly solution to the challenges posed by the current lack of standardization in diagnostic test kit labeling and packaging.

The implementation of this color coding scheme, along with accompanying text labels and icons, has the potential to improve the diagnostic testing process by minimizing errors, enhancing efficiency, and promoting consistency across different medical consumables. By addressing the limitations and drawbacks of current packaging and labeling practices, the SPECIMEN COLLECTION TYPE COLOR CODING AND ICONOGRAPHY system contributes to a more streamlined, effective, and user-friendly healthcare system.

9. Smart Phone App with Multi-Timer

This is a smart device application for tracking test results, managing profiles, and providing additional functionality related to the environmentally friendly test kits (FIG. 11-24).

The widespread adoption of smartphones and other smart devices has transformed the way people access information, communicate, and perform everyday tasks. In the healthcare sector, the rise of mobile health (mHealth) technologies has created new opportunities to improve patient care, streamline workflows, and enhance overall efficiency. Mobile applications for health-related purposes have become increasingly popular, offering users a wide range of features and functionalities to support various aspects of their healthcare journey.

One area where mobile applications can provide significant value is in the management and monitoring of diagnostic tests. Rapid diagnostic tests, such as lateral flow assays (LFAs), have become an essential tool for the detection and monitoring of various medical conditions. However, the growing number of diagnostic tests and the need for accurate timing and interpretation of test results can present challenges for healthcare professionals and end-users alike. Ensuring timely and accurate test results is crucial for effective patient care and clinical decision-making.

This ninth invention in this suite of innovations proposes a smart device application, called the “SMART PHONE APP WITH MULTI-TIMER,” designed to assist users in tracking test results, managing profiles, and providing additional functionality related to the environmentally friendly test kits. This mobile application addresses several challenges associated with the current use and management of diagnostic tests by offering users a comprehensive and user-friendly platform to monitor and interpret test results.

The SMART PHONE APP WITH MULTI-TIMER features an intuitive interface that allows users to easily track the progress of multiple diagnostic tests simultaneously, ensuring that each test is accurately timed and interpreted. This multi-timer functionality is particularly valuable in situations where multiple tests are being conducted concurrently or in rapid succession, as it reduces the likelihood of errors and enhances overall efficiency in the diagnostic testing process.

In addition to the multi-timer feature, the smart device application provides users with a range of other functionalities, including the ability to manage profiles for different patients or users, access electronic instructions for use (eIFU), and receive guidance on the proper collection and handling of specimens. By incorporating these features into a single, user-friendly platform, the SMART PHONE APP WITH MULTI-TIMER streamlines the diagnostic testing process and supports users in achieving accurate and reliable test results.

The development and implementation of the SMART PHONE APP WITH MULTI-TIMER have the potential to improve the overall efficiency and accuracy of the diagnostic testing process, while also enhancing the user experience for healthcare professionals and end-users alike. By addressing the challenges associated with the management and monitoring of diagnostic tests, this innovative smart device application contributes to a more effective and user-friendly healthcare system.

### 10. Spacket Carrier and Multi-Timer Sheet

A cardboard sheet for organizing and storing spackets, as shown in FIG. 25a, 25b.

The healthcare sector frequently relies on rapid diagnostic tests, such as lateral flow assays (LFAs), to provide timely and accurate results for various medical conditions. These tests often involve the use of small packets, called “spackets,” which contain essential reagents or consumables required for the test. Proper organization and storage of these spackets are critical for ensuring the accuracy and reliability of test results. However, the current methods for handling and storing spackets can be cumbersome, inefficient, and prone to errors, leading to potential issues in the diagnostic testing process.

This tenth invention in this suite of innovations addresses these challenges by proposing a practical and user-friendly solution for organizing and storing spackets: the “Spacket Carrier And Multi-Timer Sheet.” This cardboard sheet is designed to assist healthcare professionals and end-users in managing spackets, ensuring their proper organization, and facilitating the accurate timing of diagnostic tests.

This invention, as shown in FIGS. 25a and 25b, is a simple yet effective solution for organizing and storing spackets. It features a cardboard sheet with a series of slots or grooves designed to hold spackets securely in place, allowing users to keep track of multiple spackets simultaneously. This organized storage system helps prevent spackets from becoming misplaced, mixed up, or contaminated, which can lead to inaccurate test results and potential patient harm.

Furthermore, this invention incorporates a multi-timer feature, which enables users to track the timing of multiple diagnostic tests simultaneously. This functionality is particularly valuable in situations where multiple tests are being conducted concurrently or in rapid succession, as it helps ensure that each test is accurately timed and interpreted. By combining the spacket organization and multi-timer functionalities in a single, user-friendly solution, this invention enhances the efficiency and accuracy of the diagnostic testing process.

The development and implementation of this invention has the potential to improve the overall organization, storage, and timing of diagnostic tests, thereby contributing to more accurate and reliable test results. This innovative solution addresses the challenges associated with the current methods for handling and storing spackets and provides a practical and user-friendly alternative for healthcare professionals and end-users alike.

### 11. Complete Improved Diagnostic Test Solution

An integrated test kit containing all the redesigned components described above, as shown in FIG. 26.

The 11th invention focuses on the need for a comprehensive, efficient, and user-friendly diagnostic test solution that incorporates the various innovations and improvements described in the previous inventions. Existing diagnostic test kits often lack integration and standardization, resulting in increased costs, inefficiencies, potential errors, and confusion among healthcare professionals and end-users. The present invention aims to address these issues by providing a fully integrated and improved diagnostic test solution that combines all the innovative components and features described in the previous inventions.

This invention comprises several key components, including the redesigned Lateral Flow Immunoassay with Extended Dynamic Range (LEIQA), the LEIQAPACK environmentally friendly packaging, the LEIQA Data Management System and Electronic Instructions for Use (eIFU), the Sterile Blood Extractor (SBEx), the Specimen Collection Type Color Coding and Iconography, the Smart Phone App with Multi-Timer, and the Spacket Carrier and Multi Timer Sheet. By incorporating all these redesigned components into a single integrated test kit, as shown in FIG. 26, the invention provides a streamlined and efficient solution for diagnostic testing.

This invention offers several advantages over traditional diagnostic test kits, such as improved accuracy, enhanced user experience, reduced material waste, and more efficient storage and transportation. By combining all the innovative components and features into a single integrated solution, this invention provides a practical and user-friendly approach to diagnostic testing, making it more accessible and efficient for healthcare professionals and end-users alike.

As a summary for the background of this collection of inventions, the present invention encompasses a comprehensive and eco-friendly diagnostic testing solution that addresses the limitations and environmental concerns of traditional diagnostic tests. The following 11 inventions work together to revolutionize the diagnostic testing process:
1. Sterile Wood Abrasive Collector (SWAC): A sustainable specimen collection device made from wood, replacing traditional plastic swabs. The SWAC simplifies sample collection and reduces plastic waste, offering a more environmentally friendly alternative.
2. Pathogen Exhalate Collector (PEC): A non-invasive testing method that captures and analyzes exhaled breath for the presence of pathogens, enabling rapid testing and minimizing the need for invasive sampling procedures.
3. The Spacket: An innovative, eco-friendly buffer test tube replacement that minimizes plastic waste and simplifies reagent dispensing during testing by using small, aluminum sheet-based packets.
4. Low Environmental Impact Quantitative Assay (LEIQA): An improved lateral flow immunoassay with increased sensitivity, a broader dynamic range, and a reduced environmental footprint, providing an environmentally responsible alternative to traditional diagnostic tests.
5. LEIQA Data Management System with Integrated Electronic Instructions for Use (eIFU): A digital platform that streamlines the diagnostic testing process by managing test data and providing electronic instructions, reducing paper waste and enhancing user experience.
6. Sterile Blood Extractor (SBEx): An environmentally friendly alternative to traditional lancets and capillary tubes for blood collection, which simplifies the process and reduces waste by combining lancet and blood collection functions into a single device.
7. LEIQAPACK: An eco-friendly packaging system for medical consumables that utilizes standardized dimensions to reduce waste and improve shipping and storage efficiency, making it easier for healthcare professionals and end-users to handle and transport medical products.
8. Specimen Collection Type Color Coding and Iconography: A color coding scheme with text labels and icons indicating specimen collection methods, simplifying the identification process and reducing the risk of errors for healthcare professionals and end-users.
9. Smart Phone App with Multi-Timer: A mobile application that assists in tracking test results, managing profiles, and providing additional functionality related to the environmentally friendly test kits, enhancing user experience and ensuring accurate result interpretation.
10. Spacket Carrier and Multi-Timer Sheet: An eco-conscious solution for efficient storage and management of test components, utilizing a cardboard sheet designed for organizing and storing spackets, reducing plastic waste and improving test kit organization.
11. Complete Improved Diagnostic Test Solution: An integrated diagnostic test kit containing all the redesigned, eco-friendly components described above, delivering a comprehensive, environmentally responsible, and user-friendly diagnostic testing solution that revolutionizes the traditional testing process.

Together, these inventions form a cohesive and innovative system that addresses the need for sustainable, efficient, and accurate diagnostic testing, while minimizing the environmental impact and enhancing the overall user experience.

### BRIEF DESCRIPTION OF DRAWINGS

FIG. 1a depicts a side view and isometric view line drawing of the Sterile Wood Abrasive Collector (SWAC);

FIG. 1b is a picture of the actual SWAC next to various typical nasal and nasopharyngeal swabs;

FIG. 1c are CAD model renderings of a Pathogen Exhalate Collector (PEC) shown in various views but not to scale.

FIG. 2a depicts a side view of a typical legacy transparent plastic buffer test tube, with liquid inside, with a sealing rubber ring, and with a screw-on cap as an example of prior art;

FIG. 2b depicts an isometric view of the invention according to one embodiment of the present invention in the folded state, referred to as the “double flap bottom” version;

FIG. 2c depicts an isometric bottom view of the invention according to one embodiment in the intermediate state where an operator has started separating the bottom folds of the product, exposing the adhesive;

FIG. 2d depicts an isometric view of the invention according to one embodiment where the operator has unfolded the bottom completely and placed it on a surface;

FIG. 2e depicts an isometric view of the invention according to one embodiment where an operator has torn off the top part of the at spacket between the tear guides in preparation for use;

FIG. 2f depicts an isometric line drawing view of one embodiment of the spacket where two lines form metal memory in the aluminum causing the spacket to want to have a natural tendency to produce a volume in the center;

FIG. 2g shows the same spacket from FIG. 2f with the top part torn off exposing the naturally resulting volume inside to cause the spacket to want to open up when torn open;

FIG. 2h shows the components of one manufacturing method to put metal memory into a sheet of aluminum;

FIG. 2i shows the same as FIG. 2h but from a different viewing angle;

FIG. 2j shows two resulting formed pieces of aluminum placed symmetrically opposite from one another prior to being heat sealed together;

FIG. 2k shows the same two sheets of aluminum from FIG. 2j after they have been heat sealed together;

FIG. 2l shows an isometric view of the spacket with instructional text and graphics printed on the front;

FIG. 2m shows an isometric view of the spacket with instructional text and graphics printed on the back;

FIG. 2n shows a web User Interface (UI) of a 3 step timer in web-page web application in desktop landscape mode;

FIG. 2o shows a web User Interface (UI) of a 3 step timer in web-page web application mobile portrait mode;

FIG. 2p shows a screen capture of the timer when a slot parameter is used, such that multiple timer can be shown on one page and each timer is shown independently;

FIG. 3a depicts a second embodiment of the present invention that uses a different way of folding the material that makes up the bottom structure of the spacket, referred to as the “concave bottom” version, allowing the product to stand upright on a surface;

FIG. 3b depicts an isometric view of the second embodiment showing the top part of the present invention where an operator has torn off the top part of the at spacket between the tear guides in preparation for use;

FIG. 3c depicts an isometric semi-transparent view of the present invention tilted to show the bottom part of the spacket with focus on the concave bottom and showing how the material sheets are folded;

FIG. 4a depicts an isometric view of a typical legacy LFA cassette;

FIG. 4b depicts an isometric view of a typical legacy LFA strip that is found inside the cassette from FIG. 4a.

FIG. 5a shows a CAD model rendering of the front, side and isometric view of one embodiment of the “Low Environmental Impact Quantitative Assay” (LEIQA) targeted for detecting a multitude of antigens;

FIG. 5b shows an actual picture of an embodiment of the “Low Environmental Impact Quantitative Assay” (LEIQA) where the control line and each of the test lines are all the same color “red”;

FIG. 6 shows a CAD model rendering of the front, side and isometric view of one embodiment of the “Low Environmental Impact Quantitative Assay” (LEIQA) targeted for detecting a multitude of antibodies;

FIG. 7a shows a CAD model rendering of the front, side and isometric view of one embodiment of a dual “Low Environmental Impact Quantitative Assay” (LEIQA) targeted for detecting a multitude of antibodies on two separate LEIQAs that are fixed together with a label;

FIG. 7b shows a CAD model rendering of the front, and an isometric view of one embodiment of a dual “Low Environmental Impact Quantitative Assay” (LEIQA) specifically targeted for simultaneous detection of an HIV antigen and two distinct antibodies;

FIG. 7c shows a chart illustrating the quantity of viral RNA, antigens and antibodies as a result of an HIV infection, while illustrating time on the x-axis and quantity on the Y-axis;

FIG. 8a depicts the “double flap bottom” embodiment of a spacket torn open across the tear guides, and where a SWAC has been placed into the buffer, and where the spacket is squeezed together to increase the liquid level above the specimen collection region of the SWAC;

FIG. 8b depicts the spacket from FIG. 8a in a natural relaxed state where the LEIQA from FIG. 5a has been additionally placed into the liquid, and where some of the liquid has been consumed by the LEIQA lowering the liquid level in the spacket;

FIG. 8c shows the LEIQA kit packaging, which is an aluminum bag with various markings, and in particular a QR code that contains a URL that resolves to a web page with the instructions for us of the test kit;

FIG. 8d shows a web page which is what is shown when the QR code from FIG. 8c is scanned;

FIG. 9a shows an exploded view of a traditional lancet pen with a disposable needle inside that is typically used when collecting blood through a finger prick;

FIG. 9b illustrates the use of a traditional capillary tube to collect drops of blood from a finger after a finger prick after the use of a lancet or lancing device, such as a lancet pen.

FIG. 9c depicts an embodiment of a novel “Sterile Blood Extractor” (SBEx) shows in side, top, isometric top and isometric bottom views;

FIG. 9d depicts 3 different embodiments of the “Sterile Blood Extractor” (SBEx), that shows the use of a stainless steel needle, a ceramic element needle, and an aluminum formed needle in both top views, and side section views;

FIG. 9e shows an isometric view of a CAD model where a corner has been cut out for clarity, revealing the internal construction of one embodiment of the SBEx, depicting a stainless steel wire formed needle affixed by an adhesive to the bottom of a two part aluminum foil package that has been laminated together, and where the cavity is filled with a liquid, such as an ethanol for keeping the internal sterilized, and purged with nitrogen to avoid chemical reactions (such as oxidation) of the needle, adhesive or liquid;

FIG. 9f shows a side view of the same embodiment from FIG. 9c as a line drawing.

FIG. 9g shows one embodiment of the SBEx manufactured such that an array of products together make up convenient sheets that ease mass production, bulk packaging and distribution;

FIG. 9h shows a CAD model of one embodiment of the spacket from FIG. 3a, where a SBEx device has been affixed on the outside of the spacket where when the SBEx is engaged, the needle internal to the SBEx penetrates the spacket;

FIG. 9i, 9j, 9k shows 3 pictures in sequence on one actual embodiment of the invention where after the spacket is opened FIG. 9i, a finger is inserted FIG. 9j, and then with a second finger the SBEx is pressed to engage a needle FIG. 9k, which penetrates the spacket and cause the finger to produce drops of blood internal to the spacket and to then drip into the liquid of the spacket;

FIG. 10a shows a top view of a cardboard cutout that will later be folded up into a box;

FIG. 10b shows the box in a folded up assembled state;

FIG. 10c shows the flaps or ears from the box torn off and used as a “flap carrier” for spackets;

FIG. 10d shows a table of Specimen collector types along with the color code and color hex number for each type of collected specimen;

FIG. 10e shows a CAD model rendering of the packaging with an outer label that contains a major color blue and a magnification of a part of the label that indicates the specimen collection type, in this case “Nostril Swab”;

FIG. 10f shows a CAD model rendering of the packaging with an outer label that contains a major color red and a magnification of a part of the label that indicates the specimen collection type, in this case “Fingerprick Blood Collector”;

FIG. 10g demonstrates how a complete label set for three LEIQAPACKs can be printed on a standard A4 sheet by separating the two labels, enabling efficient label production;

FIG. 10h shows 3 LEIQA packs with 100 test kits each placed on top of each other to create an unprecedented density of product;

FIG. 11 illustrates a welcome screen of the smart device application, displaying a user interface that welcomes users upon opening the app;

FIG. 12 shows a screen listing profiles and provides options for adding more profiles, exporting profiles, and importing profiles from other patients or app users;

FIG. 13 illustrates the app menu, which allows users to change language, start camera verification of the authenticity of other patients' test results, access help and feedback menus, and navigate to a menu that links to reporting of adverse events;

FIG. 14 demonstrates the language menu, where users can choose from multiple language options, such as Norwegian, Portuguese, etc.;

FIG. 15 displays the camera verification screen of the app, which enables patients to use this function to verify the authenticity of another person's test result, without having to exchange their personal data;

FIG. 16 shows the app screen of the me-code displayed, where the me-code changes every 5 seconds to include a timestamp, ensuring that the test identification cannot be faked by taking a screenshot and then be used by the same or other users;

FIG. 17 illustrates an app screen displaying a list of already taken tests for one profile for one patient, with some tests listed as pending and other tests listed with results already obtained;

FIG. 18 presents an app screen that appears when the user taps a test to view a test result for a specific test. The quantitative results are displayed along with the positive or negative status of the test, and a code that can be scanned with the app feature shown in FIG. 15;

FIG. 19 shows the resulting app page of a person that has their test result verified by another instance of an app on another person's device;

FIG. 20 demonstrates an app screen with a graphical representation of multiple tests over time, presenting quantitative results in a visual manner;

FIG. 21 exhibits the same app screen as for FIG. 20 but with the ability to change the time scale to customize results into year, month, and custom ranges;

FIG. 22 showcases a page of the app with a timer feature that allows a patient to scan the DataMatrix of one or more tests. The app then queries the database for the specific test scanned and loads the correct development and validity time for the test, automatically starting the timer and alerting the user when the test is ready to be interpreted either by eye or a machine. A second timer starts after the first timer has expired, counting the time that the test is valid for;

FIG. 23 shows the app when the timer function is used, where the DataMatrix or QR code of a test cassette or LEIQA is scanned by the camera and then the app queries a database to get the parameters for how long the test shall be developed and how long it is valid;

FIG. 24 shows the time page of the app before any timer has been started;

FIG. 25a shows a picture of a cardboard sheet separated into 10 sections which each have a slot number, a QR code, and a rectangular groove for the spacket to be slid into.

FIG. 25b shows an isometric view of the cardboard sheet from FIG. 25a where a number of spackets have been inserted in some of the slots and where other slots are not populated with spackets for clarity of illustration.

FIG. 26 shows an illustration of the complete test kit with all components that this specification covers, and where every individual piece has been improved over existing legacy test kit components.

### DETAILED DESCRIPTION OF A MULTITUDE OF INVENTIONS

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Turning now to an overview of technologies that are more specifically relevant to aspects of the invention, as previously stated, SARS-CoV-2 is spreading rapidly around the country and around the world resulting in a large portion of the population being at risk of developing COVID-19. It is imperative to test, often repeatedly, the population for SARS-CoV-2, and present test kits are inefficient, wasteful, and costly.

Antigen testing is typically used to test for SARS-CoV-2 nucleocapsid proteins. Typically, a nasopharyngeal or nasal swab is inserted into the nose of a patient and samples of the patient's epithelial cells are collected on the swab as it is removed from the patient's nose. The swab is then placed into a tube containing a buffer. The buffer typically consists of detergent, a lysing component to open the sample cells to get cytoplasm out of the cells, and a buffer agent. A pipette is then used to draw an analyte from the tube and place a few drops of analyte onto the analyte pad of an LFA strip in a test cassette. An antibody bound with a color particle on a test line of the LFA strip will combine with any antigen present in the analyte, releasing the color particle to color the test strip to indicate the presence of the antigen, i.e. a positive test result. This process and these components generate a lot of medical and plastic waste. It is also costly to package each of these components together into each test kit, which also increases weight thus transportation costs.

Antibody testing is performed in a similar fashion, but with an antigen or receptor protein bound to a color particle used as the marker. Antibody testing is traditionally done using centrifuged venous blood (serum), although some newer types of tests can use blood from a finger prick as described below. Whether antibody or antigen, both are chemical markers—a piece of chemistry that is being looked for. Biological markers such as proteins may also be looked for, such as in pregnancy tests. Also, non-organic chemical markers may be looked for such as arsenic or chlorine in pools, for example. All are chemical markers.

Finger pricks may also be used for antibody and antigen testing. A lancet is used to draw the blood from the finger to produce blood. A capillary straw having a ten microliter mark draws blood which is then expelled by the straw onto an LFA strip. The buffer used when testing venous blood contains an anticoagulating agent and an anti red blood cell filtration component, so that mostly only serum makes its way up the LFA strip to the testing site on the LFA strip. Again, this system also has additional medical waste in the form of the lancet for pricking the finger and the capillary straw and their associated packaging.

### 1. The Sterile Wood Abrasive Collector (SWAC)

Turning now to the first aspect of this invention, which is an improvement, alternative or replacement of nasal and nasopharyngeal swabs, which are designed to collect specimens from the nasal cavity and nasopharynx for diagnostic testing, such as the detection of respiratory pathogens like influenza and SARS-CoV-2. The swabs typically consist of two parts: the swab tip, which is used to collect the specimen, and the handle, which allows for easy manipulation during the collection process.

Swab Tips are typically made from one or more of the following materials;
Rayon, which is a synthetic fiber made from regenerated cellulose, derived from wood pulp or other plant sources. Rayon swab tips are soft, non-abrasive, and have good absorption properties, making them suitable for specimen collection.
Polyester, which is made from synthetic fibers, which are non-absorbent and less likely to cause interference with diagnostic tests. These swab tips are widely used due to their durability and compatibility with various testing platforms.
Dacron, which is a type of polyester material that is often used in swab tips, is chemically inert and has a low likelihood of interfering with the test results.
Flocked nylon is made by attaching short nylon fibers to a solid core through a process called flocking. These swab tips have a high surface area, which enables efficient specimen collection and release.
Cotton, is made from natural cotton fibers. Although they are soft and absorbent, cotton swabs are not ideal for some diagnostic tests due to potential interference from natural proteins and other cellular components.

Swab Handles are typically made from one or more of the following materials;
Polystyrene, which is a common plastic material used for making swab handles.
It is lightweight, rigid, and easy to mold into various shapes.
Polypropylene, which is another widely used plastic for swab handles. It offers good chemical resistance, flexibility, and is relatively low-cost.
Aluminum, or other metals which are used in swabs that require more strength to navigate the nasal passage.

In conclusion, nasal and nasopharyngeal swab tips and handles are made from various materials, including synthetic fibers (e.g., rayon, polyester, Dacron, and flocked nylon), natural fibers (e.g., cotton), and different types of plastics (e.g., polystyrene and polypropylene) or metals (e.g., aluminum). The choice of material depends on factors such as the intended use, compatibility with diagnostic tests, cost, and availability.

Both long and short swabs available today may appear frightening to patients, and especially children, in the anticipation that much of the entire swab length will be inserted into their nose causing discomfort and pain.

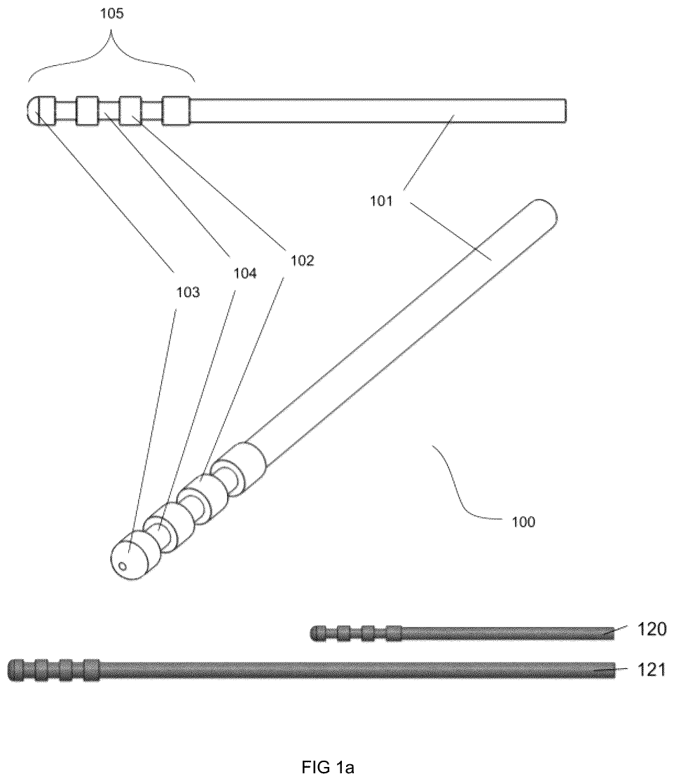

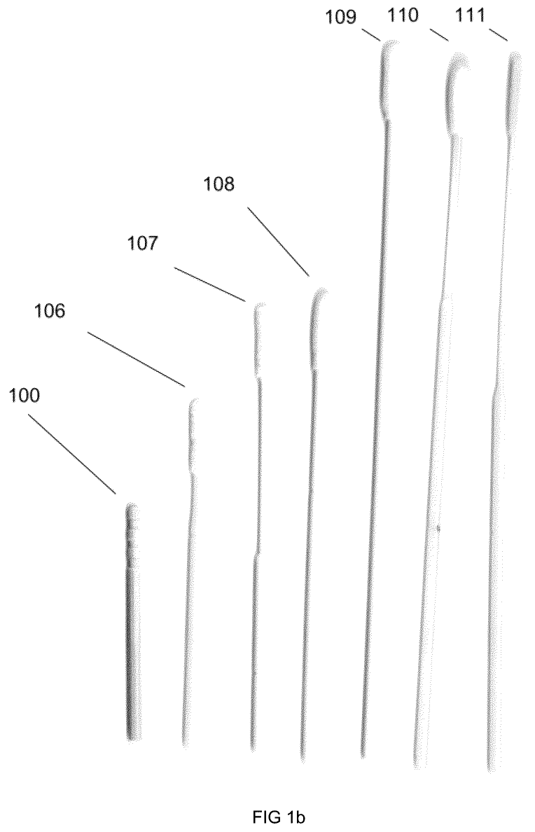

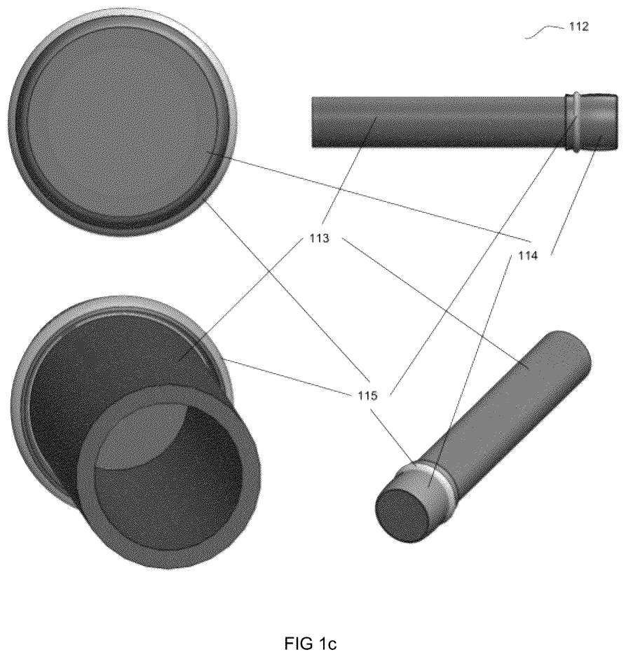

The first (1st) aspect of the present invention, the Sterilized Wood Abrasive Collector (SWAC) shown in FIG. 1a, is directed toward a novel, environmentally friendly, recyclable, and sustainable specimen collector device. The SWAC is designed as a single-piece construction, with two integrated parts: a cylindrical handle 101 and a collector head 105. The collector head is composed of a series of alternating larger 102 and smaller 104 cylinders that create surfaces for efficient specimen collection, retention, and deposition. The invention addresses the shortcomings of traditional swabs and offers improved specimen collection and release for rapid test applications.

Describing now the various features of the SWAC.

The SWAC Cylindrical Handle 101; The handle part of the SWAC is a cylindrical structure made from a single piece of biodegradable, renewable material, such as bamboo, sustainably harvested wood, or other rapidly renewable sources. The handle is designed for easy grip and manipulation by the user during specimen collection, ensuring proper control and comfort.

The SWAC Collector Head 105; The collector head consists of a series of alternating larger and smaller cylinders that are machined or molded integrally with the handle, creating a single unit. The larger cylinders function to scrape off epithelial cells, while the smaller cylinders collect and retain the specimen. The geometrical arrangement of the cylinders is designed to maximize surface area and provide optimal scraping, collection, and retention of specimens.

Use of the SWAC for Scraping and Collection; The larger cylinders in the collector head effectively scrape off epithelial cells from the nasal cavity, potentially containing pathogens, while minimizing the collection of mucus. This design ensures that the SWAC efficiently collects specimens with minimal contamination from mucous or other unwanted materials. The tip of the collector head, comprising the first of the larger cylinders, a rounded front 103 facilitates the SWAC to be inserted into orifices safely and with less resistance than if the tip was sharp or flat.

Describing now features and advantages of the SWAC over traditional swabs:
SWAC Retention; The smaller cylinders of the collector head are designed to retain the specimen securely during transportation. The geometrical arrangement and the inherent properties of the wood material contribute to the retention of the collected specimen, minimizing the risk of specimen loss or contamination.
SWAC Release; Upon deposition into a liquid buffer, the collected specimen is readily released from the smaller cylinders due to the hydrophilic nature of the wood material. This property ensures efficient transfer of the specimen into the liquid buffer, facilitating subsequent analysis.
SWAC Sterilization; The SWAC is sterilized using environmentally friendly methods, such as gamma irradiation or ethylene oxide, to ensure that the device is safe for use and poses no risk to patients or healthcare professionals.
SWAC Environmental Benefits; By using a single piece of renewable, biodegradable material, the SWAC offers significant environmental advantages over traditional swabs. The use of rapidly renewable sources, such as bamboo or sustainably harvested wood, reduces the carbon footprint, waste generation, and overall environmental impact of the device.
SWAC Recyclability and Sustainability; The SWAC's single-piece construction from biodegradable material facilitates recycling and reduces waste. The sustainable sourcing of the wood material ensures that the production of SWACs does not contribute to deforestation or habitat loss, promoting environmental responsibility.

The SWAC in summary, the Sterilized Wood Abrasive Collector (SWAC) is a single-piece specimen collector device with a cylindrical handle and a collector head composed of alternating larger and smaller cylinders. This invention offers a more environmentally friendly, recyclable, and sustainable alternative to traditional swabs, while efficiently collecting, retaining, and releasing specimens for rapid test applications.

Describing now the technical features of the material used in the SWAC and in particular, its chemical and physical properties.

The difference in the release of biological tissue samples collected with a SWAC made from bamboo versus a traditional nylon swab into a reagent buffer containing detergent can be attributed to the chemistry and physics of the materials, as well as their respective surface properties.

The SWAC Hydrophilicity; Bamboo is a naturally hydrophilic material due to the presence of hydroxyl (—OH) functional groups on its cellulose fibers. These hydroxyl groups can form hydrogen bonds with water molecules, resulting in a strong affinity for water. In contrast, nylon is a synthetic polymer composed of repeating amide linkages, and its overall hydrophilicity is lower than that of bamboo. When the bamboo-based SWAC comes into contact with the reagent buffer, the hydrophilic nature of the material facilitates the interaction between the specimen and the buffer, promoting efficient release.

The SWAC Surface Roughness; Bamboo fibers have a more porous and rougher surface than nylon, due to their natural structure and composition. This increased surface roughness provides better adhesion for the collected biological tissue sample. However, when the bamboo-based SWAC is immersed in a reagent buffer containing detergent, the detergent molecules can interact with the bamboo surface, reducing surface tension and weakening the adhesion between the sample and the fibers. This facilitates the release of the sample into the buffer. On the other hand, the smoother surface of nylon swabs may lead to a more tightly bound specimen, making it harder for the detergent to effectively release the sample.

The SWAC Swelling Properties; When immersed in a liquid, bamboo fibers can swell due to the absorption of water molecules. This swelling can cause a slight deformation of the bamboo fibers, which may help dislodge the biological tissue sample and facilitate its release into the reagent buffer. In contrast, nylon has a limited capacity for swelling, making it less effective in releasing the sample upon contact with the buffer.

The SWAC Detergent Interaction; Detergents in the reagent buffer are amphiphilic molecules, having both hydrophilic (polar) and hydrophobic (non-polar) regions. The hydrophilic regions can interact with the hydroxyl groups present on the bamboo surface, while the hydrophobic regions can interact with the biological tissue sample. This dual interaction helps to solubilize the sample and effectively release it from the bamboo fibers. In the case of nylon swabs, the interaction between the detergent and the swab material is weaker due to the lower hydrophilicity of nylon, making it less efficient at releasing the sample.

SWAC technical feature conclusion; The superior release of biological tissue samples collected with a bamboo-based SWAC into a reagent buffer containing detergent, as compared to a traditional nylon swab, can be attributed to bamboo's hydrophilic nature, surface roughness, swelling properties, and enhanced interaction with the detergent. These factors contribute to more efficient and effective release of the specimen for subsequent analysis.

The following describes two methods on how the SWAC may be manufactured, although it should be understood by those skilled in the art that other methods could be used to produce a functionally equivalent product.

A first manufacturing method of the SWAC involves machining it from a solid piece of wood, similar to how toothpicks are made. The process includes the following steps; Material selection where choosing a suitable type of wood or bamboo that is sustainable, fast-growing, and has desirable properties for specimen collection. Cutting and shaping, where the wood is cut into cylindrical blanks of appropriate size for the SWAC handle. Machining, where the cylindrical blanks are machined using specialized cutting tools or lathes to create the integrated handle and collector head, including the series of alternating larger and smaller cylinders. Sanding and smoothing, where the machined SWACs are sanded and smoothed to remove any rough edges or splinters, ensuring a comfortable and safe experience for the user during specimen collection.

A second manufacturing method for the SWAC describes molding a mixture of wood particles and biodegradable adhesive, utilizing wood particles, sawdust, and other waste products from the production of the SWAC itself during the first method, or other wood products, combined with a biodegradable adhesive to form the final product. The process includes the following steps; Material preparation, where wood particles, sawdust, and larger wood splinters are filtered and ground into smaller particles with a maximum size appropriate for the SWAC production process. Adhesive selection, where a biodegradable adhesive is selected to bind the wood particles. Possible adhesives include starch-based glues, lignin-based binders, protein-based adhesives, or even sugar-based adhesives. Mixing, where the wood particles are mixed thoroughly with the biodegradable adhesive to create a homogeneous mixture. Molding, where the mixture of wood debris and adhesive is deposited into SWAC-shaped forms or molds. Heat and pressure application, where the filled molds are subjected to heat and pressure, which not only helps in shaping the SWAC but also partially sterilizes the product due to the application of heat. The heat and pressure also cause the adhesive to cure, binding the wood particles together into a solid, durable structure. Demolding and finishing, where after the curing process, the SWACs are removed from the molds, and any excess material or rough edges are trimmed or sanded as needed.

Both methods of SWAC production offer advantages in terms of sustainability and environmental impact, with the first method utilizing a solid piece of wood and the second method making use of wood waste products and a biodegradable adhesive. Choosing the most suitable method will depend on factors such as the availability of raw materials, production costs, and manufacturing capabilities.

A visual and structural comparison between the SWAC and traditional swabs are shown in FIG. 1b, where the SWAC 100 is substantially shorter than traditional swabs 106-111, and has an appearance similar to that of a “toothpick” which most people are familiar with and find much less frightening. Like toothpicks, the SWAC can be made from various types of wood, but bamboo is preferred due to its abundant availability across the world, and its use as a sustainable eco-friendly renewable material. The picture in FIG. 1b shows the following swabs; 106 a 75 mm swab from Taiwan company TSTC, 107 a 95 mm swab from Taiwan company TSTC, 108 a 100 mm swab from Medico Technology Company Co. Ltd. in China, 109 a 150 mm swab from manufacturer Puritan in the USA, 110 a 150 mm FLOQSwab from manufacturer COPAN in the USA, and 111 a 155 mm swab from manufacturer Puritan Medical Products in the USA.

Describing now some of the SWAC specimen collection use cases, where the multitude of larger 102 and smaller 104 cylinders produce round edges that abrasively scrape off epithelial cells from the skin, mucosa, or gum line, and where the specimen attaches to the wood and the ridges in the wood thus collecting tissue potentially containing pathogens from a patient, an animal, or a specimen from nature such as growth of biological colonies on a rock. Due to the rigid structure of the SWAC it may also be pushed into the flesh or soft tissues, and thus act as an equivalent of a biopsy instrument, as the front tip is rounded to allow easy entry into soft tissue. Those skilled in the art of specimen collection will appreciate that traditional swabs are too soft in structure to collect whereas the SWAC is rigid enough to sustain the force pressure applied without deforming.

The tip of the SWAC 103 is rounded to ease insertion and reduce friction as the SWAC is pushed and rotated back and forth while touching the area of the patient that the specimen is collected from. The SWAC may also be used to collect specimens from the urinary tract, vagina, anus, ear canal, or other orifices as well as skin rashes and pus from wounds. The SWAC may be similarly used on animals such as fish, bats, birds, and farm animals.

FIG. 1a also shows two photorealistic CAD renderings of two embodiments of the SWAC in various sizes. In one embodiment, the SWAC 120 is specifically designed for human nostril sampling. This SWAC features a smaller and more flexible design, which allows for a comfortable and efficient collection of specimens from the nasal cavity. The size and shape of the collector part are optimized to facilitate easy navigation within the human nostril, minimizing discomfort while maximizing the effectiveness of specimen collection.

Furthermore, the invention also encompasses a larger, thicker SWAC 121 designed for sampling and specimen collection of orifices in animals, particularly in the context of pathogen detection. This larger SWAC is suitable for sampling various orifices, such as the mouth, nose, vagina, rectum, and reproductive tracts in animals like cattle, pigs, sheep, goats, and poultry. The enhanced size and durability of this SWAC enable effective specimen collection from these farm animals, facilitating the monitoring and management of potential pathogens and ensuring the health and safety of both animals and humans.

### 2. The Pathogen Exhalate Collector (PEC)

Respiratory pathogens are a significant global health concern, causing numerous illnesses and deaths each year. Rapid and accurate detection of these pathogens is crucial for preventing the spread of infectious diseases and improving patient outcomes. Current diagnostic methods often rely on expensive, complex equipment and consumables, which may not be accessible or affordable for low-income populations or under-resourced healthcare settings. In addition, many diagnostic consumables generate substantial waste and contain materials that can be harmful to the environment. Therefore, there is an urgent need for inexpensive, equitable, recyclable, and environmentally friendly diagnostic consumables that enable rapid disease detection from respiratory pathogens.

Respiratory pathogens include viruses such as influenza, coronaviruses (including SARS-CoV-2), respiratory syncytial virus (RSV), adenoviruses, and rhinoviruses. Bacterial pathogens include tuberculosis (Mycobacterium tuberculosis), Streptococcus pneumoniae, Haemophilus influenzae, and Bordetella pertussis. Respiratory parasites comprise Pneumocystis jirovecii, Paragonimus westermani, and Strongyloides stercoralis, among others.

The Pathogen Exhalate Collector (PEC) is designed to provide a simple, inexpensive, and environmentally friendly method for collecting and analyzing exhaled breath, mucus, or sputum from patients to detect respiratory pathogens. The PEC comprises two main components: a cardboard or paper tube and a filter material, which are joined together using an environmentally friendly adhesive.

The cardboard or paper tube serves as the primary structure of the PEC. It is designed to be held by the patient or a healthcare worker during use. The tube can be made from recyclable materials, such as craft paper, corrugated cardboard, or other biodegradable materials, which are lightweight and cost-effective. In some embodiments, the tube can be coated with a thin layer of biodegradable plastic or wax to provide additional moisture resistance and improve durability during use.

The filter material is designed to capture and collect pathogens present in the exhaled breath, mucus, or sputum of the patient. It can be made from a variety of materials, including non-woven polypropylene, polyester, or other synthetic or natural fibers with suitable pore sizes and filtration properties. The filter material may also be treated with antimicrobial agents, such as metal nanoparticles (e.g., silver, copper, or zinc), or other substances to inactivate trapped pathogens and reduce the risk of contamination during handling.

The environmentally friendly adhesive used to attach the filter material to the cardboard or paper tube is designed to be water or liquid-soluble and biodegradable. This adhesive can be made from various chemical compositions, including but not limited to natural polymers like starch, cellulose, or chitosan, or synthetic polymers like polyvinyl alcohol (PVA) or polylactic acid (PLA). The adhesive may also include additives, such as plasticizers, cross-linking agents, or other substances, to enhance its performance and compatibility with the filter material and tube.

When the PEC is immersed in a buffer liquid, the adhesive dissolves, allowing the filter material to separate from the tube and release the trapped pathogens or other biological materials into the buffer liquid. This facilitates further diagnostic analysis and compatibility with various diagnostic assays for the detection of respiratory pathogens, including viruses, bacteria, and parasites. The use of environmentally friendly, biodegradable, and recyclable materials in the PEC minimizes its environmental impact and supports sustainable healthcare practices.

Now describing in detail the CAD model rendering in FIG. 1c, the Pathogen Exhalate Collector (PEC) 112 is shown in various views, although not to scale. The PEC 112 comprises a cardboard or paper tube 113, which can be held by the patient or a healthcare worker. The patient places their lips around the tube and exhales, producing mucus or sputum that enters the tube. The filter material 114, made from a fabric or material similar to that used in surgical masks, captures and collects the exhaled pathogens. A ring of glue, adhesive, or similar chemical 115, composed of environmentally friendly, water or liquid-soluble, and biodegradable substances, attaches the filter material 114 to the cardboard or paper tube 113.

When the PEC 112 is immersed in a buffer liquid, the adhesive 115 dissolves, allowing the filter material 114 to separate from the tube 113 and fall into the buffer. This process releases the trapped pathogens or other biological materials into the buffer liquid, facilitating further diagnostic analysis and compatibility with various diagnostic assays.

### 3. The Standing Packet (Spacket)

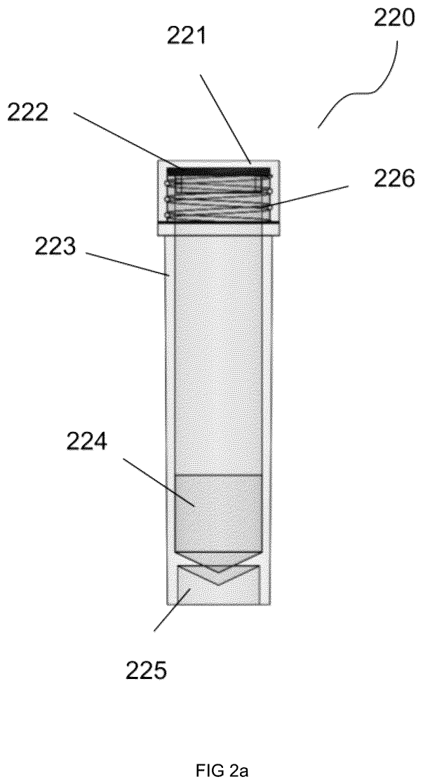

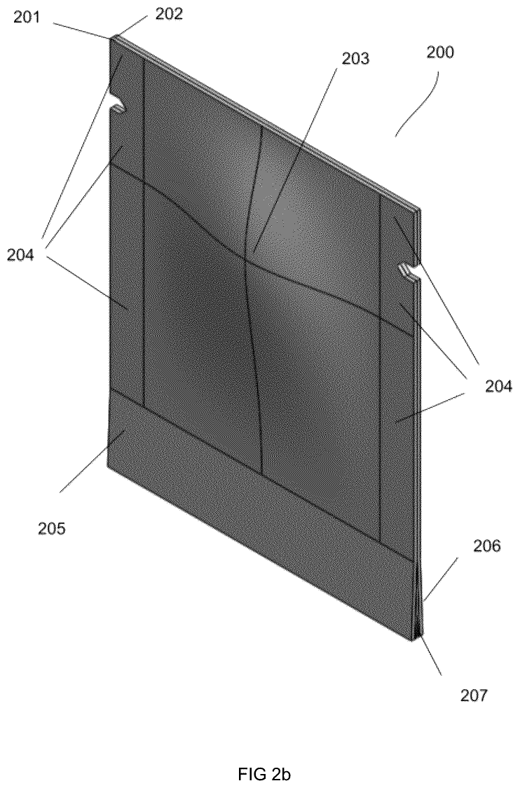

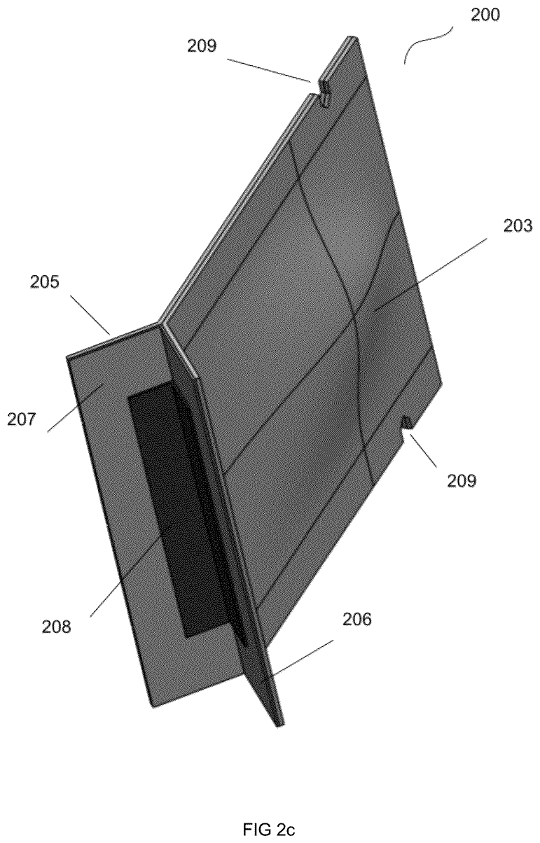

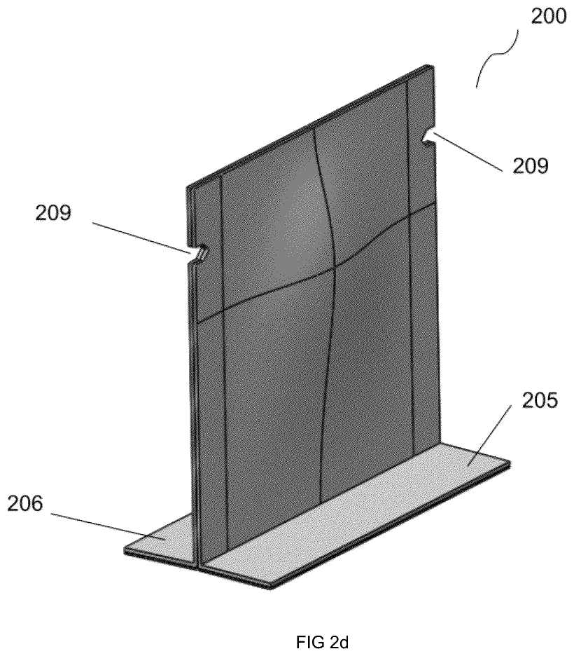

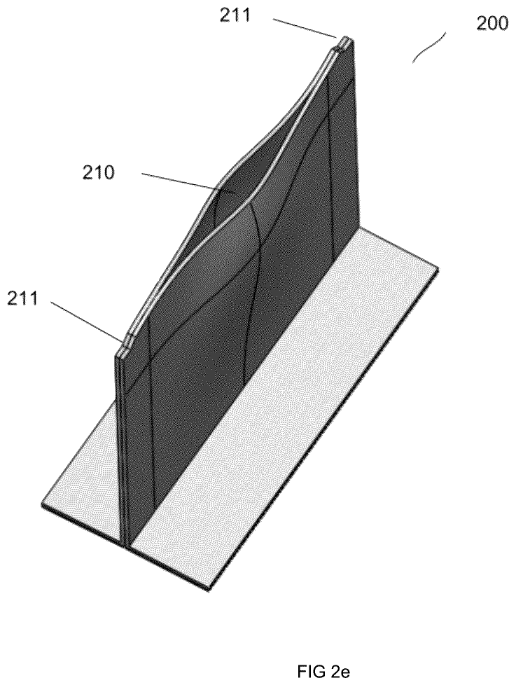

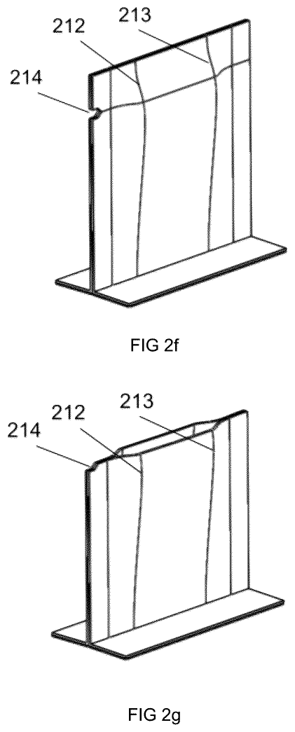

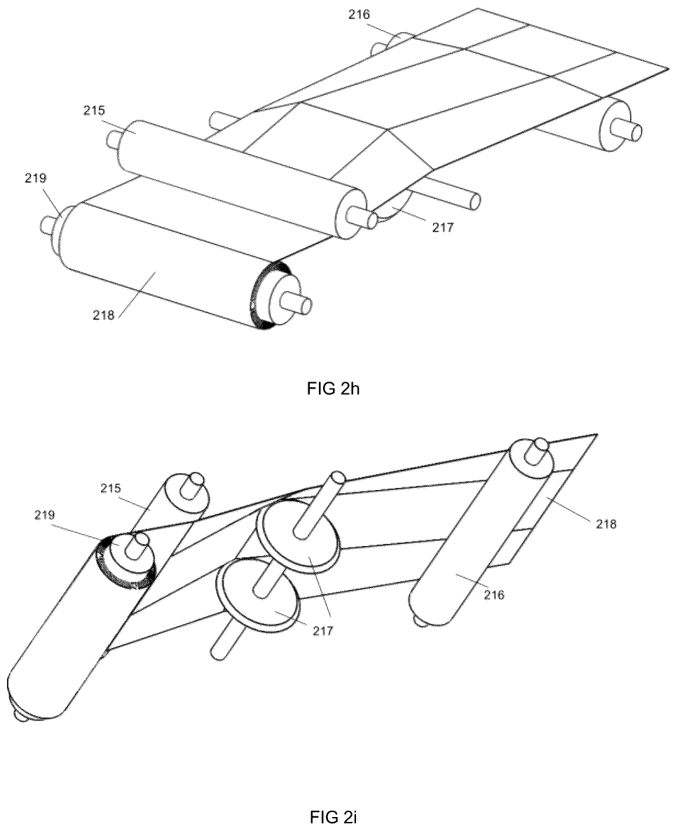

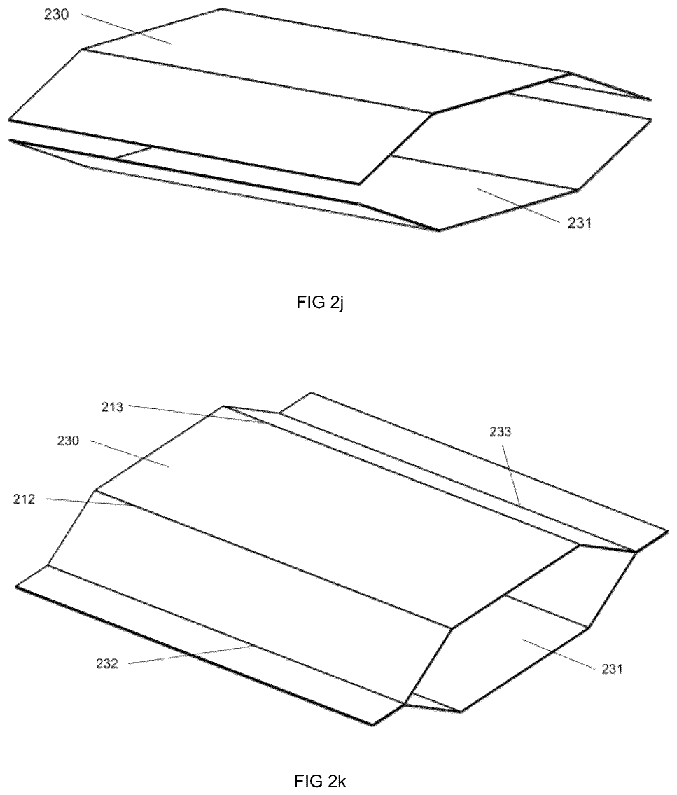

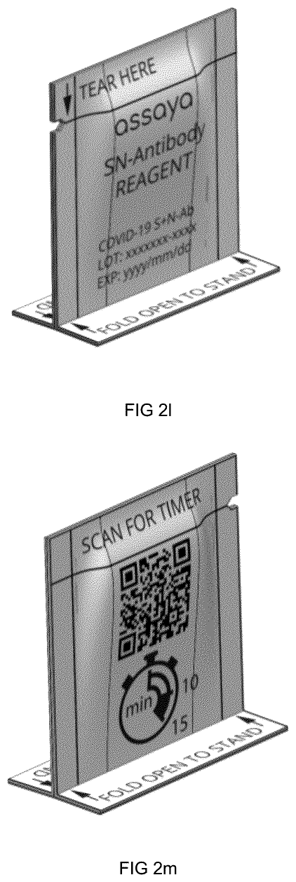

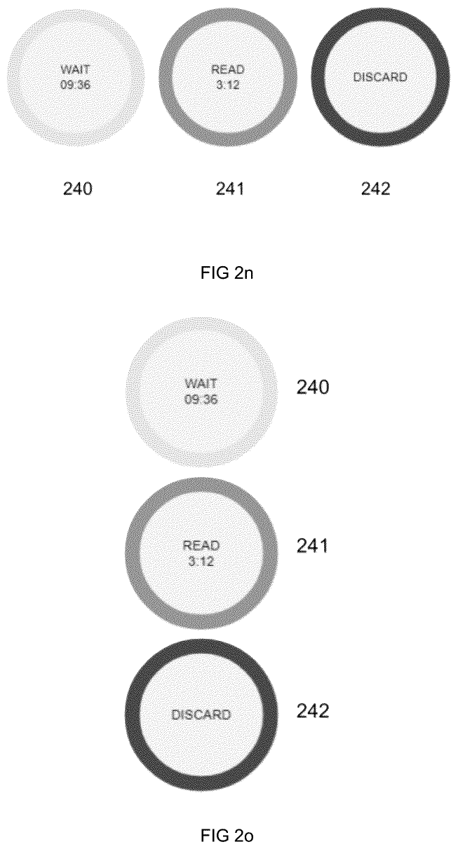

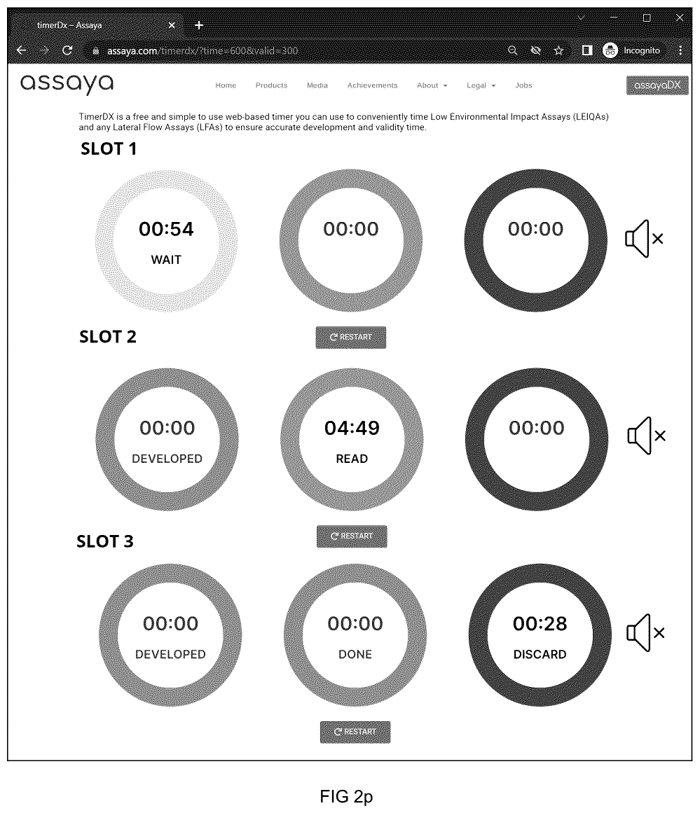

Turning now to an overview of the second (2nd) aspect of the invention, that replaces the current plastic test tube containing a liquid shown in FIG. 2a that depicts a semi-transparent front view 220 of a traditional buffer test tube 223 with a top cap 221 containing a liquid 224 that may act, with one or more functions combined, as a reagent, lysing agent, detergent, or inhibit other chemical functions required to prepare the sample for subsequent reactions with an LFA testing strip. The test tube 223 and cap 221 typically contain the materials polyethylene (“PE”), polypropylene (“PP”) or polymethyl methacrylate (“PMMA”). The buffer cap 221 sealed by a rubber ring, or o'ring, 222 is rotated onto the structure to seal the vessel during transportation and storage. Threads 226 allow for screwing the cap 221 onto the main vessel body 223. At the bottom of the vessel, a cavity 225 creates a void underneath the vessel, which creates an internal downward facing volume where particles, mucus and debris can accumulate.

At least three embodiments are hereinafter disclosed that replaces the utility and function of the product 220 with a more environmentally friendly and less polymer containing product 200 and 300, that hereinafter will be referred to as a “spacket”, which is short for “standing packet”, and that substantially contains thin commercially available and easily recyclable aluminum foil that is folded in a rolling process that combines foil from two reels and that additionally adds a third aluminum foil segment to form a device that can stand upright on a table top during use.

While a two part aluminum foil packet would not have the ability to stand upright on its own, adding the extra fold in the bottom allows the spacket the ability to stand vertically upright, which is needed for the practical use case of such devices. The embodiments described hereinafter that both add the feature of the devices standing upright, but it should be understood that those skilled in the art could make variations of this design that would allow similar utility while preserving the spirit of this invention.

The first embodiment called “double flap bottom” of the spacket 200 teaches the use of a rectangular aluminum sheet 207 between two aluminum sheets 205 and 206 that are pressed together while being heated, causing a seam 204 to form that permanently adheres the aluminum sheets 201 and 202 to each other forming a vessel capable of containing a liquid that does not leak out through the seams even if some pressure is applied to the bubble 203 that forms in the center volume of the spacket. A layer of sticky glue 208, similar to that found on sticky-notes or POST-IT™ notes, may be placed on the bottom foil 207 that is folded shut during manufacturing FIG. 2a, and that can be opened by an operator as seen in FIG. 2b, and then folded out completely flat as seen in FIG. 2c to allow the spacket to be adhered to a surface, such as a table. FIG. 2d shows the spacket with the two flaps 205 and 206 completely unfolded to make up a flat bottom that allows the product to balance on a table top or surface. Two tear marks 209 on either side of the spacket indicate and facilitate the spacket to be torn open by an operator as seen in FIG. 2e, where an opening 210 is produced by tearing the spacket open between tear marks 211 such that the liquid contained substantially in the bottom of the spacket is exposed through the opening 210, which allows for the subsequent process of placing diagnostic products into the spacket while it is standing upright.

A second embodiment shown in FIG. 2f is a variation of the first embodiment with an improvement in the product's ability to open up and remain open during the use of the product. The improvement is achieved by adding two folds 212 and 213 into the aluminum foil prior to adhering the two rolled sheets that make up the spacket. FIG. 2g shows the spacket after the top has been torn off at the tear mark 214. Note that there may be only one tear mark on one side 214, or there might be two tear marks on either side 209.

This second embodiment of the invention teaches a method for forming a three-dimensional shape in a sheet of aluminum by inducing metal memory bends through a rolling process shown in FIG. 2h and FIG. 2i, which subsequently leads to the creation of a volume when two sheets are combined and heat-sealed together. In this inventive process, an aluminum sheet 218 on a roll 219 is passed over tension providing rollers 215 and 216 between two sharp-edged wheels 217, each applying a force that imparts a metal memory bend to the aluminum sheet 218. Upon removal of the tension, the metal memory bends induce a natural tendency for the aluminum sheet to form bends and create a three-dimensional shape.

It should be understood and appreciated that other arrangements of the sharp wheels can be used in combination with another set of sharp edge wheels on the opposite sides to realize the desired function of metal memory fold without deviating from the spirit of the invention. Another method would be to fold the metal sheet at the edges from both sides to make a memory impression into the aluminum that produces the desired effect of the spacket wanting to spring open when the top part is torn off.

FIG. 2j shows the resulting aluminum sheet 230 combined with a second similarly processed sheet 231, and the two sheets are heat-sealed together along their edges 232 and 233. Due to the metal memory bends 212 and 213 introduced during the rolling process, the combined sheets shown in FIG. 2k exhibit a natural inclination to form a shape with a volume between them, creating a structure that is suitable for various applications, such as packaging, enclosures, or other forms of containers. This innovative method offers a simple and efficient means of shaping aluminum sheets in a controlled manner, resulting in the formation of volumetric structures with minimal external force or intervention.

FIG. 2l and FIG. 2m show front and back views of the spacket with simple brief instructions for use printed on both sides. Instructions specify that the bottom flaps should be folded to stand, and a text “tear here” indicates where the top part of the spacket shall be torn off for use. A QR code is printed on one side that can be scanned with a smart device that links to a website where a timer application starts when the web page is opened. The novel aspect of this invention is to use a QR code that, when scanned, triggers the opening of a web page containing a web application with a countdown timer in three distinct states. The QR code encodes a URL, such as “https://assaya.com/timerdx?time=10&valid=5”, where “?time” and “?valid” represent the respective durations of the first and second states in seconds. The URL may also use minute and second syntax such as “?tim_min=10&tim_sec=0&valid_min=5&valid_sec=0” to represent the time for development and validity of the timers to use this format.

Upon scanning the QR code, the user is directed to the web page, which automatically initiates a first countdown timer. This web page is responsive, meaning that as the browser window scales from wide to narrow, the featured items shift position and orientation on the page to reflect the optimal use of the available display area on both desktop, tablet and mobile, and in case where the mobile device is tilted to landscape mode. One graphical representation of these 3 timer states for portrait and landscape modes are illustrated respectively in FIG. 2n and FIG. 2o.

In another embodiment shown in FIG. 2p, the page contains multiple timers where each timer accepts a slot parameter indicating which slot among a multitude of slots where each timer is tracked individually on one page. The web page may also produce audio, such that audible voice commands for which slots are ready can be played when tests are ready to read or as a warning of tests where the valid time has expired and the test in that slot is no longer valid. The 3 states shown in FIG. 2p demonstrate a situation where the test in SLOT 1 has started developing, and in SLOT 2 where the test is ready to read, and in SLOT 3 where the test is overdue and should be discarded. A mute icon for each slot allows the user to suppress audio signals from each slot, and a reset button for each slot allows the user to restart the test, in which the typical scenario is that another fresh test is placed into that slot to be developed.

The timer initially enters the “Developing” state labeled “WAIT” 240, wherein the countdown starts from the specified “time” value and counts down to zero. During this phase, the timer is displayed with a yellow color, analogous to the wait at a traffic light about to turn green. Once the first timer reaches zero, it transitions to the “Ready to Read” state labeled “READ” 241, which is green analogous to a traffic light go state. The read time state starts counting down from the specified “valid” value. During this phase, the timer is displayed with a green color, indicating that the test is ready for interpretation. When the valid to read green timer reaches zero, it enters the “Expired” state labeled “DISCARD” 242, which is read analogous to a traffic light stop state, where the timer continues counting in negative numbers, and both the timer and caption are displayed in red, signaling that the test is no longer valid for interpretation, and where the timer continues to indicate how long the timer has been invalid for. Tests that have not been during the validity period and after the expired timer has lapsed shall be discarded.

Lateral Flow Assays (LFAs) are diagnostic tests used to detect the presence or absence of a target analyte, often proteins or molecules, in a sample. These tests rely on the principles of capillary action and the interaction between the sample and test components, such as antibodies, to produce a visual result. However, LFAs are only valid during a specific, limited time window due to the inherent nature of the chemistry involved in their development. Over time, the chemical reactions and interactions taking place within the LFA may continue to progress, leading to changes in the test results. For instance, the binding of the target analyte to its corresponding antibodies can become saturated, causing false positive or false negative results. Additionally, other components of the sample or the assay, such as enzymes or substrates, may degrade or undergo unwanted reactions, further impacting the test's accuracy. Moreover, environmental factors such as temperature and humidity can significantly influence the rate and extent of these chemical reactions. High temperatures can accelerate the reaction rates, while low temperatures may slow them down. This can alter the optimal development time for the assay, making it challenging to accurately interpret the results outside of the recommended time window. Similarly, high humidity levels can cause the test components to degrade or become contaminated, potentially leading to erroneous results. Considering these factors, it is crucial to read and interpret the results of LFAs within the specified time window to ensure the reliability and accuracy of the test outcomes. Reading the test results outside of this time frame increases the likelihood of obtaining false positive or false negative results, which can lead to incorrect diagnoses or treatment decisions.

While LFAs sometimes can show control and test lines for weeks or even months after they are developed, they are for the reasons stated above technically not valid. When LFAs are read too long after they are developed the result should be considered invalid and the time window of the ability to read the results reliably is considered to be expired.

The next series of figures show the web application that is triggered by the scan of the QR code from the spacket while passing parameters through the URL.

The use of smart devices such as smartphones and tablets to read the QR code of the spacket eliminates the need for actual physical timers that are hardware devices containing plastics and other non recyclable materials. Also, in environments that require lots of test kits to be developed in parallel, the use of

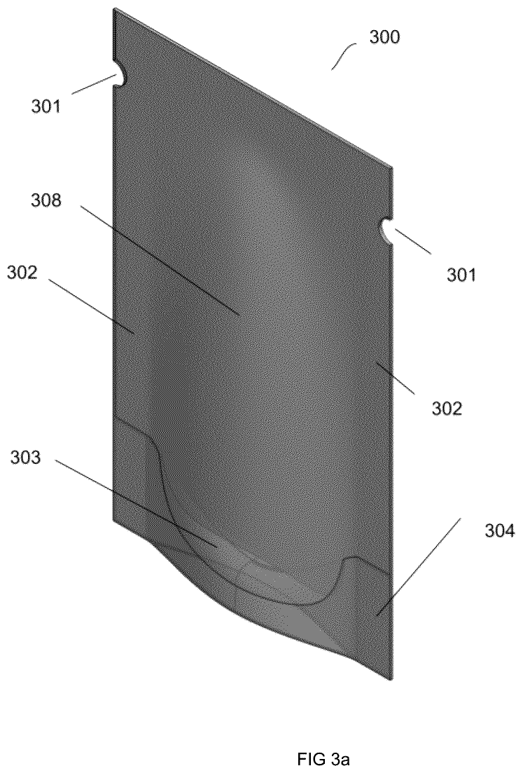

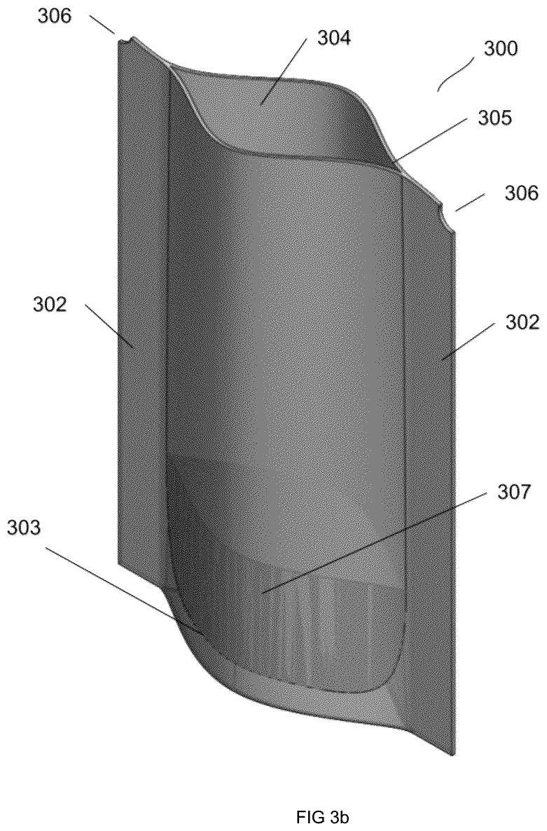

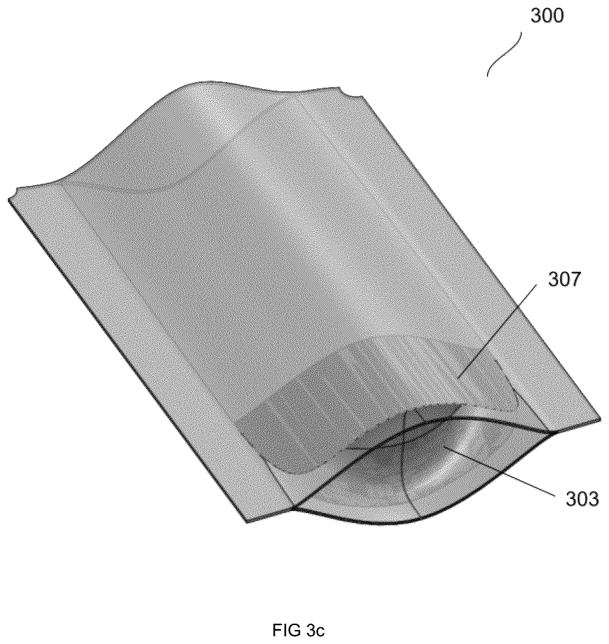

A third embodiment FIG. 3a called “concave bottom” of the spacket 300 teaches the use of a rectangular sheet that forms a rounded concave cup like surface 303 at the bottom of the spacket as shown in FIG. 3b. and 3c and that contains a liquid 307. Similarly to the first embodiment 200 the second embodiment 300 is formed from pressing aluminum sheets together and applying heat, which forms seams 302 sealing the spacket. An opening 304 results when the top part of the spacket is torn off between tear marks 301, resulting in tear marks after tearing 306.

The various embodiments related to the spacket described previously all disclose the method of heat sealing two or more aluminum sheets together. The aluminum sheets used for this purpose typically use a type of polymer of specialized adhesive which is coated or applied on one side of the aluminum sheet. Typical coatings used for this purpose may be polyethylene (PE), polypropylene (PP), polyester (PET), surlyn, vinyl or poly vinyl chloride (PVC), all which are polymers that are technically difficult and cost prohibitive to separate from the aluminum during recycling.

This specification teaches the use of polylactic acid (PLA), which is a biodegradable and compostable polymer derived from renewable resources such as cornstarch or tapioca roots or sugarcane as an environmentally friendlier alternative to the polymers described above.

### Background Information—Legacy LFAs

A Lateral Flow Assay (LFA), also known as a Rapid Diagnostic Test (RDT), is a simple, fast, and portable diagnostic tool used to detect the presence or absence of a target analyte, such as pathogens, hormones, or biomarkers, in a liquid sample. LFAs are widely employed in medical diagnostics, environmental monitoring, and food safety testing.

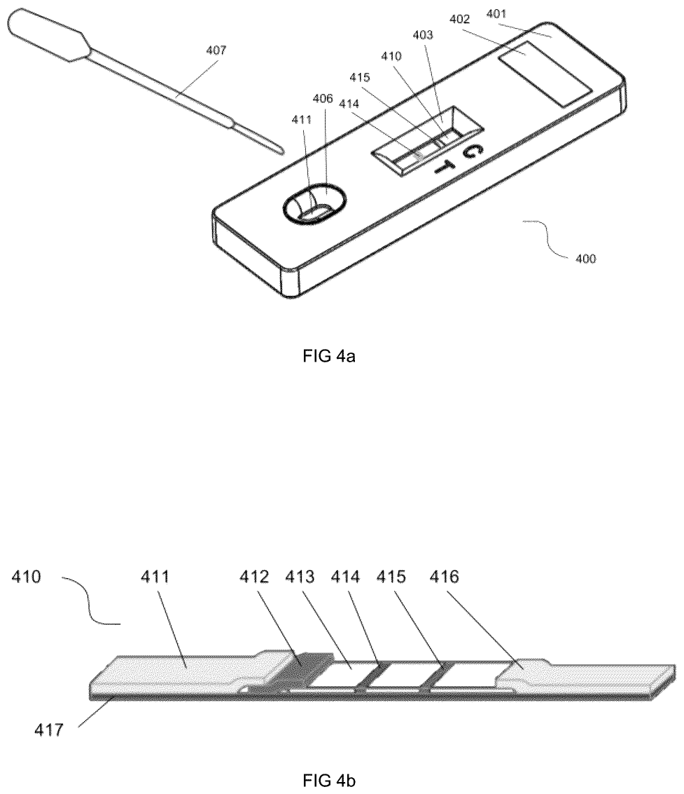

A typical LFA 400 consists of an outer plastic cassette as seen in FIG. 4a with an internal LFA strip 410 as seen in FIG. 4b. The construction of an LFA comprises several key components that work together to enable the assay to function effectively. These components include:

Sample pad 411: This is the starting point of the assay where the liquid sample is applied. The sample pad is typically made of porous materials such as cellulose, glass fiber, or polyester, which facilitate the absorption of the sample and its subsequent movement through capillary action. The pad can also be treated with chemicals or surfactants to enhance the flow properties and reduce non-specific binding.

Conjugate pad 412: This pad is impregnated with detection reagents, usually conjugated to reporter particles such as colloidal gold, latex beads, or fluorescent dyes. The detection reagents can be antibodies, antigens, or other molecules designed to bind specifically to the target analyte. When the liquid sample flows through the conjugate pad, the conjugates are rehydrated and mobilized, allowing them to interact with the target analyte present in the sample.

Reaction membrane 413: The reaction membrane is a critical component of the LFA, as it hosts the test 414 and control line(s) 415. The membrane is usually made of nitrocellulose or other materials with suitable pore size and flow properties. The test line contains immobilized capture molecules (e.g., antibodies or antigens) specific to the target analyte. The control line, located downstream of the test line, contains molecules that capture the conjugates, ensuring that the assay is working correctly. As the sample flows through the reaction membrane, target analyte-conjugate complexes form and are captured by the test line, producing a visible signal, usually in the form of a colored line.

Absorbent pad 416: This pad is placed at the end of the LFA strip to absorb excess liquid and ensure a consistent flow of the sample through the assay. It is typically made of cellulose, glass fiber, or other porous materials.

Backing material 417: The entire LFA strip is assembled on a non-reactive, waterproof backing material, such as plastic or adhesive-coated laminates, providing support and maintaining the structural integrity of the assay.

Housing/cassette 401: The LFA strip is often placed in a plastic housing or cassette to protect it from contamination, damage, and facilitate sample application and result interpretation. The test cassette often has a raised area for marking 402, a window 403 to read the results of the LFA strip 410 that contains one or more indicators 415, and a window to place the drops from an analyte with a pipette 407 onto the sample pad 411 of the strip 410.

In a typical LFA, the user applies the liquid sample (e.g., blood, urine, or saliva) to the sample pad 411. The sample migrates through the conjugate pad 412, where it mixes with the conjugates. The mixture then flows through the reaction membrane 413, where specific binding events occur at the test line 414 and control line 415, producing visible signals. The result is usually available within 5 to 30 minutes, making LFAs a popular choice for point-of-care diagnostics and field testing.

### 4. Low Environmental Impact Quantitative Assay (LEIQA)

Turning now to a third (3rd) aspect of this invention, which is an improvement, alternative or replacement of LFAs (or RDTs) with a Low Environmental Impact Quantitative Assay” (LEIQA). A LEIQAs is a device that is manufactured according to the LEIQA specification, which is a document shared with manufacturers under non-disclosure agreement, that describes in technical detail how LEIQA compatible products should be designed and manufactured.

LEIQA devices combines inventive features that are improvements over legacy LFAs in the following important ways:

LEIQAs eliminate the need for a plastic housing/cassette 401 that are typically used in LFAs, thus providing a lighter in weight, volumetrically lower, and more environmentally friendly alternative to LFAs, that require less and lighter packaging and reduced shipping and transportation costs.

LEIQAs have a DataMatrix, QR-code, barcode 502 602 702 printed on a sheet 507 607 718 that covers the absorbent pad, thus indicating the type of assay along with other information such as manufacturing date, lot code, expiration date, and a unique serial number for the individual assay, which allows the product to be tracked all the way from production through distribution, use and disposal.

This sheet 507 607 718 that covers the absorbent pad 518 616 719 can further contain a text 503 603 703 that indicates the test and assay type, which may be abbreviated or replaced with a short form code to fit on the label with a readable size font. The text ABCR-Ag may for example be a short form for Influenza A (A), Influenza B (B), Covid-19 (C), and Respiratory Syncytial Virus (R) and Antigen (Ag).

LEIQAs are designed to be placed into the spacket containing reagent buffer, thus eliminating the need for a pipette to transport drops from a legacy buffer test tube to the sample pad of a legacy LFA. Eliminating the pipette further reduces single use plastic waste, reduces weight, volume, and packaging, but also improves the sensitivity of the assay by allowing the full amount of specimen to be exposed to the sample pad, since using a pipette will inevitably leave most of the diluted specimen in the legacy buffer test tube, and leave some of the diluted specimen in the pipette itself, thus reducing the overall sensitivity of the assay.

LEIQAs are widthwise thinner and lengthwise longer than typical legacy LFAs, resulting in a slower development and longer reaction time, something that further improves sensitivity.

LEIQAs being physically longer than legacy LFAs also allows for more test lines to be located in the active region of the reaction membrane, allowing a single assay to produce results for more than one test, as an example a single test could be developed from a single specimen sample that detects COVID-19 (SARS-CoV-2), Influenza A, Influenza B, Syncytial Virus (RSV), Streptococcus pneumoniae, Legionella pneumophila, Mycoplasma pneumoniae, Bordetella pertussis, and Streptococcus pyogenes.

LEIQAs being capable of detecting a multitude of markers substantially increase the practical sensitivity since legacy LFAs typically require a single specimen swab per test which for every swab reduce the amount of available epithelial cells in the collected specimen.

LEIQAs are optimized for machine reading of the individual test lines over legacy LFAs as the length of the assay and position of the test lines are specified and designed to be located in the optimal most sensitive region for the camera, least distortion of lens, most uniform light spread by the diffuser, and position of light sources that comprises an instrument used for reading and analyzing such assays.

LEIQAs have an indicator that clearly shows which end of the assay goes into the machine reader indicated by an arrow 501, 601, 701 unlike LFAs, which typically do not have any visual marking or indicator for this.

LEIQAs have a clear indicator for which end of the assay goes into the spacket containing reagent buffer by a liquid drop 514, 614, 717 in contrast to LFAs which use a pipette 407.

LEIQAs have a color coated section 512, 612, 715, 716 at the one end that is introduced to the specimen or sample, where the color indicated what type of specimen the particular LEIQA is for, unlike LFAs which has no such indication, and require the operator to look that up in an Instruction For Use (IFU) document. Some examples of specimen types are exhalate, saliva, nostril-swab, throat-swab, blood (whole), blood (serum), blood (plasma), blood (menstruation), blood (WBC/platelets), pus/wound/abscess, sputum/phlegm, vaginal swab, penile swab, urine sample, water sample, fecal/stool collection sample, and gingival (gum) swab.

LEIQAs being manufactured regardless of type to a standard specification for size, marking, and positioning of features allows for consistent manufacturing quality and faster development time of new assays types unlike legacy LFAs for which there are no set specifications and where every manufacturer produces their own LFAs to their own requirements.

Two or more LEIQAs can be combined together 700 in the same device to allow for testing for more marker types from a single sample specimen unlike legacy LFAs, which require a plastic housing to combine multiple LFAs strips together, and in such case require each individual LFA to receive its own analyte.

While most legacy LFAs use a plastic sheet backing material 417, LEIQAs use paper, cardboard or a combination laminate of paper and cardboard that has been treated to be hydrophobic through the treatment with water-repellent substances, which create a protective layer that prevents water absorption. There are various methods and materials available for this purpose, such as:
a. Wax coating: Applying a thin layer of wax, such as paraffin or beeswax, to the surface of paper or cardboard can make it hydrophobic. This can be done by melting the wax and carefully brushing it onto the surface, or by using wax paper and an iron to transfer the wax to the paper or cardboard. Make sure the entire surface is coated evenly, and allow it to dry completely.
b. Silicone spray: Silicone sprays are commercially available and can be used to create a hydrophobic layer on paper or cardboard. Simply spray the silicone evenly onto the surface, following the manufacturer's instructions, and allow it to dry. Silicone sprays are generally more durable than wax coatings, but may be less environmentally friendly.
c. Alkyl ketene dimer (AKD): AKD is a synthetic sizing agent used in the paper industry to create water resistance. It can be applied to paper or cardboard during the manufacturing process or as a post-treatment. However, AKD might not be easily accessible for household use.
d. Fluoropolymer-based coatings: Some commercially available coatings, such as those based on fluoropolymers (e.g., Teflon), can provide a hydrophobic surface when applied to paper or cardboard. These coatings are generally more durable and water-resistant than wax or silicone but can be more expensive and less environmentally friendly.
e. Nanotechnology-based coatings: Some hydrophobic coatings use nanotechnology, such as nano-silica or other nanoparticles, to create a water-repellent surface. These coatings can be applied by spraying or brushing onto the paper or cardboard surface and allowing them to dry.

### LEIQA Types

LEIQAs are categorized into 3 main categories which are now explained and specified in detail, where a first category substantially detects antigens (AG) and a second category substantially detects antibodies (AB) and a third category is a combination of the two first categories.

### LEIQA for Antigens (AG)

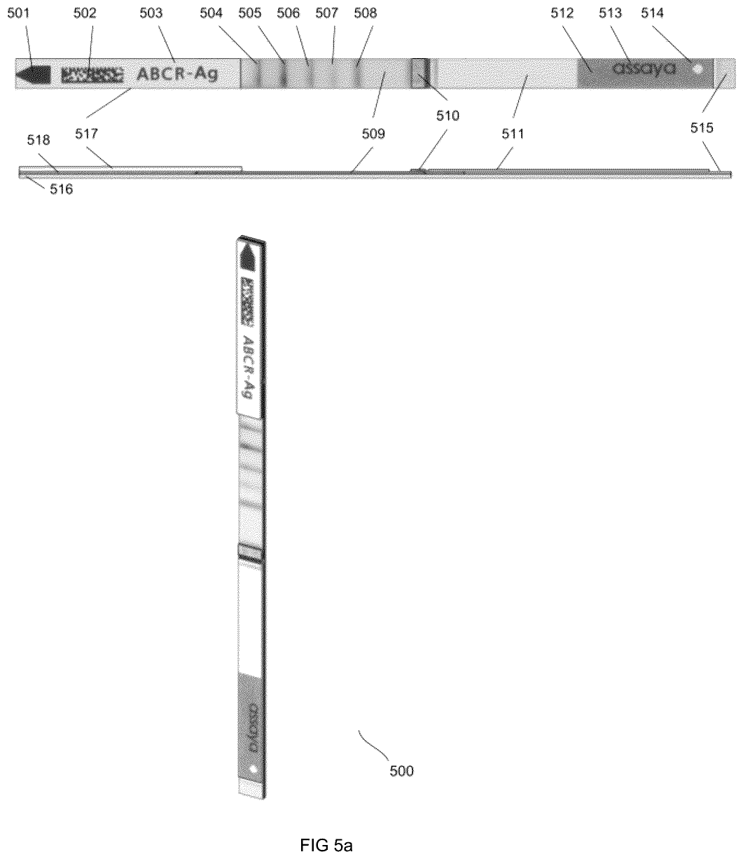

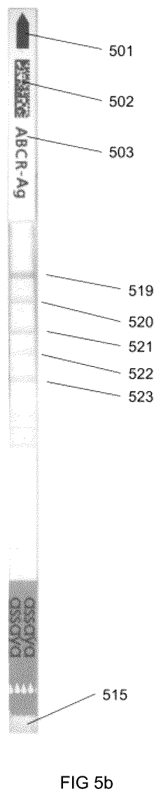

FIG. 5a shows a first type of LEIQAs “LEIQA-AG” 500 which is constructed to substantially detect AntiGens (AG or AGs). Antigens are molecules, usually proteins or polysaccharides, that can trigger an immune response when detected by the immune system. They are often found on the surface of pathogens such as bacteria, viruses, fungi, and parasites, but can also be present on non-pathogenic foreign substances. Antigens are recognized as foreign by the immune system and are responsible for stimulating the production of specific antibodies. Each antigen has unique structural features, called epitopes, that allow it to be specifically recognized and targeted by the immune system. In the context of disease diagnosis, antigens are often used as markers to determine the presence of an infection or other medical condition.

The LEIQA-AG 500 consists of a backing material 516 usually made from paper, cardboard or a paper/cardboard combination that has been treated to be hydrophobic through the application of a liquid-repellent substance as previously explained.

A sample pad 515, similar in function to that used on legacy LFAs 411, but lengthwise longer and widthwise smaller to facilitate a slower reaction time, thus more sensitive as earlier described, and with the ability to have more area to put various chemicals in sequence to increase the performance and utility of the LEIQA. Some possible chemicals that could be used include:
a. Surfactants: These chemicals, such as Tween 20, Triton X-100, or other nonionic surfactants, can be used to improve the wettability and flow properties of the sample pad, ensuring uniform distribution of the sample and preventing non-specific binding.
b. Blocking agents: To prevent non-specific binding and reduce the background noise in the assay, blocking agents such as bovine serum albumin (BSA), casein, or polyvinyl alcohol (PVA) can be used.
c. Preservatives: Antimicrobial agents, such as sodium azide or ProClin, can be incorporated into the sample pad to prevent microbial growth, ensuring the stability and shelf-life of the assay.
d. Buffers: The sample pad may be treated with buffers, such as phosphate-buffered saline (PBS) or Tris-buffered saline (TBS), to maintain a stable pH and ionic strength, ensuring optimal conditions for the assay's performance.
e. Chelating agents: Ethylenediaminetetraacetic acid (EDTA) or other chelating agents may be used to bind divalent cations, which can interfere with the binding interactions in the assay.
f. Reducing agents: To prevent oxidation and maintain the stability of sensitive analytes or detection reagents, reducing agents such as dithiothreitol (DTT) or β-mercaptoethanol can be used.
g. Enzyme inhibitors: In cases where the sample contains proteases or other enzymes that may degrade the target analyte or interfere with the assay, enzyme inhibitors can be incorporated into the sample pad.
h. Stabilizers: For assays involving labile or sensitive analytes, stabilizing agents, such as trehalose, sucrose, or polyethylene glycol (PEG), can be used to enhance the stability of the target analyte or detection reagents during storage and transport.

These chemicals can be used individually or in combinations, depending on the specific needs of the LEIQA. The optimal formulation will need to be determined through experimentation and optimization to ensure the best performance, sensitivity, and specificity of the assay.

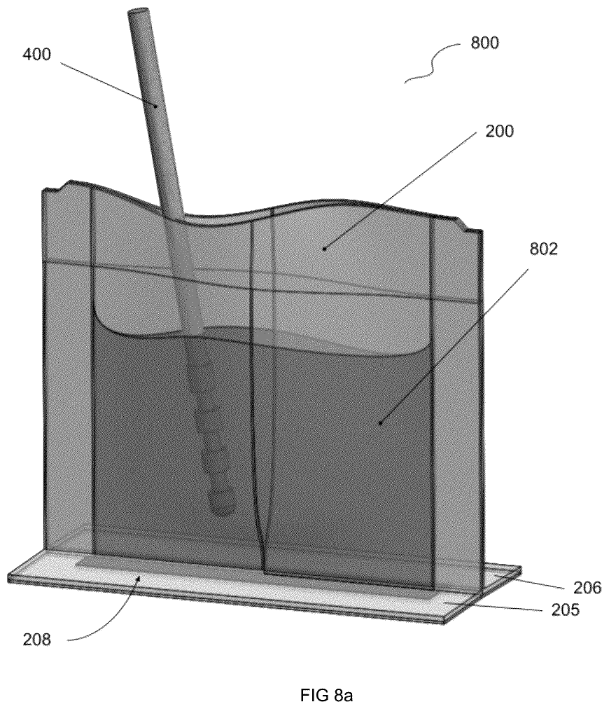

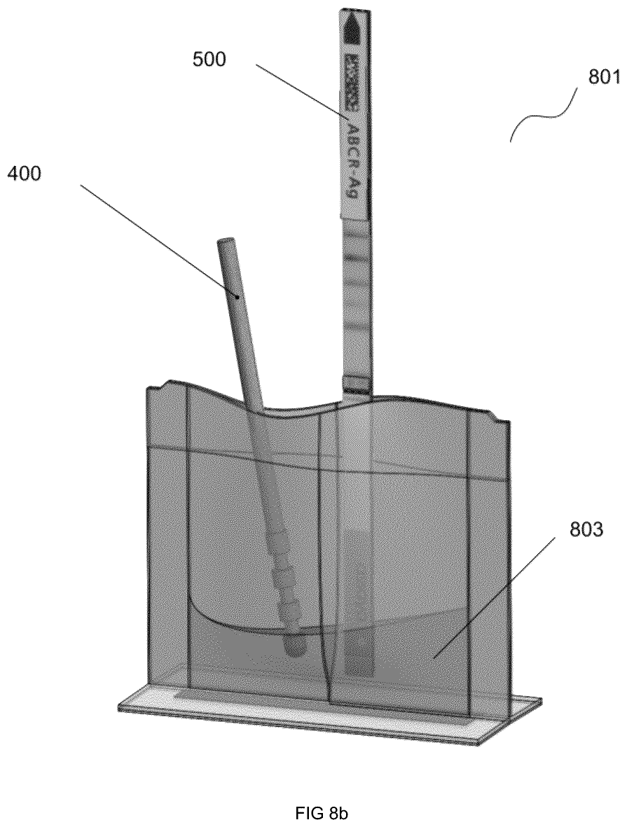

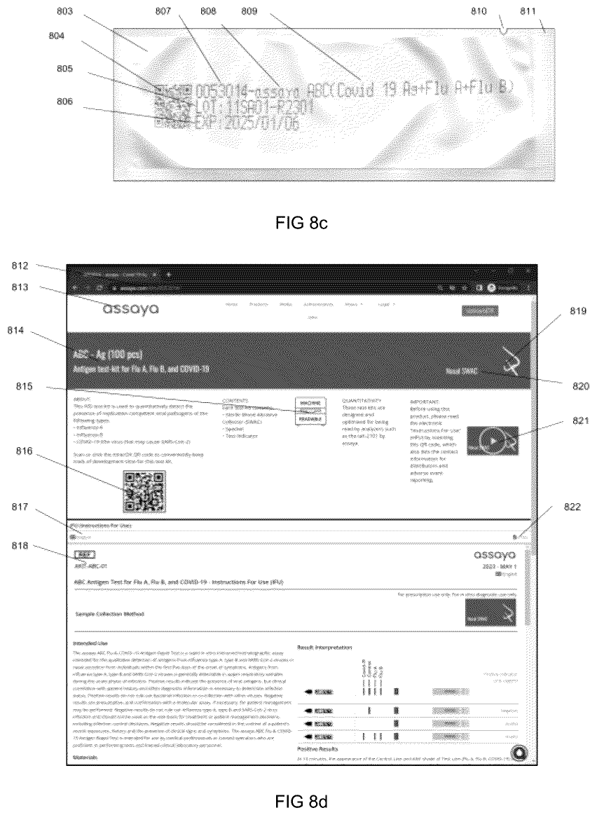

The sample pad 515 may be covered by a sheet 511, typically made from hydrophobically imprinted paper or cardboard, that comprises a “Specimen Collection Identification Color” (SCIC) 512 imprinted with a brand indicator 513, and a liquid drop icon 514 indicating which end of the assay should be placed into the liquid, which would typically be a spacket as illustrated in FIG. 8b.

The conjugate pad 510 is similar in function to the conjugate pad of a legacy LFA 412, but with some several important differences and enhancement as described subsequently. In current legacy LFAs, the conjugate pad typically contains detection reagents conjugated to reporter particles. Here are some commonly used components in the conjugate pad of legacy LFAs:
a. Detection reagents: These are usually antibodies or other bio-recognition molecules (e.g., aptamers, antigens, or enzymes) that specifically bind to the target analyte. The choice of detection reagent depends on the target and the desired assay format (e.g., sandwich, competitive, or indirect).
b. Reporter particles: Detection reagents are conjugated to reporter particles, which generate a detectable signal upon binding to the target analyte. Common reporter particles include:
c. Colloidal gold nanoparticles or Latex beads
d. Fluorescent dyes or quantum dots
e. Paramagnetic particles
f. Enzymes (e.g., horseradish peroxidase or alkaline phosphatase)
g. Stabilizers and preservatives: To maintain the stability and shelf-life of the conjugated reagents, chemicals such as sucrose, trehalose, bovine serum albumin (BSA), or polyvinylpyrrolidone (PVP) can be used. Preservatives, such as sodium azide or ProClin, may be added to prevent microbial contamination.

While LEIQAs may also use one or more of these components, they additionally present the use or one or more of the following components to enhance the capability of testing for more chemical and biological markers, several additional components could be considered:
a. Multiplexed detection reagents: A mixture of different detection reagents, each specific to a unique target analyte, that are incorporated into the conjugate pad. This would improve simultaneous detection of multiple targets in a single test.
b. Advanced reporter particles: Novel reporter particles, such as upconverting phosphors or plasmonic nanoparticles, used to provide more sensitive and quantitative signals, enabling the detection of low-abundance targets or the simultaneous measurement of multiple targets with minimal signal interference.
c. Affinity-enhancing agents: Chemicals that can enhance the binding affinity or specificity of the detection reagents, such as molecular chaperones or engineered binding proteins, could be incorporated into the conjugate pad to improve the assay's sensitivity and specificity.
d. Enzymatic amplification: By incorporating enzymes that can catalyze signal amplification reactions, such as nucleic acid amplification enzymes (e.g., in nucleic acid-based LFAs) or enzymatic substrates for colorimetric or chemiluminescent detection, the sensitivity and dynamic range of the assay could be improved.
e. Smart materials: The use of stimuli-responsive materials (e.g., thermoresponsive or pH-responsive polymers) in the conjugate pad could enable on-demand release of the detection reagents or fine-tuning of the assay conditions to optimize performance for each target analyte.

The selection and incorporation of these additional components will depend on the specific requirements of the multi-marker LFA, and their optimal formulation will need to be determined through experimentation and optimization, and those skilled in the art of LFA design and construction would appreciate that one or more of the markers listed above could be used in combination and that chemical equivalents or biosimilars could be used to further enhance LEIQAs over legacy LFAs.

The reaction membrane 509, being probably the most critical part of the LEIQA, is also fundamentally similar to the reaction membrane of a legacy LFA 413, but with some important distinctions. Similar to the sample pad 515 and the conjugate pad 510, the reaction membrane in LEIQAs are widthwise shorter and lengthwise longer for several important reasons, specifically promoting a slower development time causing a longer reaction time, which promotes higher sensitivity. LFAs and LEIQAs are typically manufactured in rectangular sheets where the chemicals are printed on the various substrates in parallel as the sheet rolls under a series of nozzles that releases a liquid onto the substrates much like if several ball point pens were fixed in space and where a paper rolled under them forming a series of parallel lines of ink. The sheet is then cut with a knife or a stamping tool into individual test strips. Because the width of each LEIQA test strip is reduced as compared to legacy LFAs, the amount of chemicals deposited per strip is reduced, allowing for more test strips per sheet, and less use of chemicals per strip which ultimately results in lower per unit manufacturing cost.

In LFAs the reaction membrane, typically made of nitrocellulose or other porous materials, serves as the platform where the assay's key interactions and signal generation occur. The membrane is usually treated with chemicals to immobilize test and control lines, and sometimes, to enhance assay performance, and some common components used in LFA reaction membranes are:
a. Test and control line reagents: The test line typically contains immobilized capture molecules, such as antibodies or antigens, that specifically bind to the target analyte. The control line often contains secondary antibodies or other molecules that bind to the detection reagent, serving as an internal quality control to ensure the assay is functioning correctly.
b. Blocking agents: After immobilizing the test and control line reagents, the membrane is usually treated with blocking agents, such as bovine serum albumin (BSA), casein, or polyvinyl alcohol (PVA), to reduce non-specific binding and minimize background noise.

LEIQAs aim to test for more chemical and biological markers with better sensitivity and performance than legacy LFAs, and may utilize several additional components individually or in combination such as those described below:
a. High-performance membranes: Novel materials or modified nitrocellulose membranes, such as cellulose acetate, polyethersulfone, or polyvinylidene fluoride (PVDF), with improved capillary flow properties, enhanced protein binding capacity, or lower non-specific binding could be used to improve assay sensitivity and reproducibility.
b. Precision patterning: Advanced printing or patterning techniques, such as inkjet printing or photolithography, that immobilize multiple capture reagents with high spatial resolution, enabling the simultaneous detection of multiple targets in a single test.
c. Signal amplification: Incorporating signal amplification strategies, such as enzyme-based amplification systems (e.g., horseradish peroxidase or alkaline phosphatase with appropriate substrates), catalytic nanomaterials (e.g., gold, silver, or platinum nanoparticles), or hybridization chain reaction (using target-specific DNA or RNA probes), that enhance the assay's sensitivity and dynamic range, allowing for the detection of low-abundance targets.
d. Advanced detection methods: Employing advanced detection methods, such as fluorescent or electrochemical readouts (e.g., organic dyes, quantum dots), in combination with the appropriate reporter particles or transducers (e.g., redox-active molecules, electrocatalytic nanoparticles), that improve the assay's sensitivity, quantitation, and multiplexing capabilities.
e. Optimization of membrane chemistry: Fine-tuning the chemistry of the membrane, such as by using affinity-enhancing agents (e.g., molecular chaperones, engineered binding proteins), smart materials (e.g., thermoresponsive or pH-responsive polymers), or advanced surface coatings (e.g., self-assembled monolayers, polymer brushes), that optimize the binding interactions and assay conditions for each target analyte, resulting in improved performance.

The selection and incorporation of these additional components will depend on the specific requirements of the LEIQA. These chemicals and materials can be used individually or in combinations to improve the performance, sensitivity, and multiplexing capabilities of LEIQAs. The optimal formulation and integration of these components will depend on the specific requirements of the assay and will need to be determined through experimentation and optimization.

Similar to legacy LFAs, the example test lines 504 506 507 508 and the black control line 505 in the reaction membrane of a LEIQA play a crucial role in capturing and detecting target analytes, as well as providing internal quality control. The chemistry used in producing these lines involves immobilizing specific biomolecules on the reaction membrane, typically made of nitrocellulose or other porous materials, where the test line is composed of capture molecules, such as antibodies, antigens, or aptamers, which are specific to the target analyte. These molecules are immobilized on the membrane in a defined location, forming a narrow band or line. The process of immobilizing these molecules typically involves one of the following methods:
a. Passive adsorption: Nitrocellulose membranes possess a natural affinity for proteins. The capture molecules are applied to the membrane using a dispenser or printer (e.g., a BioJet dispenser), which deposits a small volume of a concentrated solution containing the capture molecules onto the membrane surface. The membrane is then dried, allowing the proteins to adsorb to the nitrocellulose fibers.
b. Covalent attachment: Capture molecules can also be covalently linked to the membrane surface through reactive groups on the molecules and the nitrocellulose fibers. This method requires pre-treating the membrane with cross-linking agents (e.g., glutaraldehyde) or modifying the capture molecules with reactive functional groups (e.g., amine or thiol groups).

The control line 505 consists of secondary antibodies or other molecules that bind to the detection reagent, regardless of the presence or absence of the target analyte. The purpose of the control line is to serve as an internal quality control, ensuring that the assay is functioning correctly. The control line is produced using the same immobilization methods mentioned above (passive adsorption or covalent attachment).

After immobilizing the test and control line reagents, the membrane is typically treated with blocking agents to reduce non-specific binding and minimize background noise. Common blocking agents include bovine serum albumin (BSA), casein, or polyvinyl alcohol (PVA). The membrane is then dried and assembled into the LEIQA device, along with other components such as the sample pad, conjugate pad, and absorbent pad. The chemistry used in producing the test and control lines in a LEIQA reaction membrane involves immobilizing specific capture molecules, typically through passive adsorption or covalent attachment, followed by blocking to minimize non-specific binding.

LEIQAs provide several environmentally friendly advantages over legacy LFAs while for producing test and control lines while maintaining or enhancing the sensitivity and utility of the assay over traditional LFAs:
a. Surface functionalization: For improved immobilization efficiency and orientation of capture molecules on the test and control lines, functionalize the reaction membrane with eco-friendly surface chemistry:
   i. Self-assembled monolayers (SAMs) of alkanethiols or silanes with reactive functional groups (e.g., carboxyl, amine, or thiol groups) for covalent attachment of the capture molecules. These chemicals can be used in low concentrations to minimize environmental impact.
   ii. Biodegradable or biocompatible polymer brushes or hydrogels with reactive functional groups or specific binding moieties to enhance the immobilization and orientation of capture molecules.
b. Signal amplification: Incorporate green signal amplification strategies directly at the test and control lines to enhance the assay's sensitivity:
   i. Enzyme-linked capture molecules: Conjugate eco-friendly enzymes, such as horseradish peroxidase (HRP) or alkaline phosphatase (AP), to the capture molecules, enabling enzyme-catalyzed amplification reactions at the test and control lines.
   ii. Biodegradable or non-toxic catalytic nanomaterials: Immobilize green catalytic nanomaterials, such as biodegradable polymer nanoparticles, at the test and control lines to amplify the signal generated by redox reactions or other catalytic processes.
c. Molecular imprinting: Create molecular imprints of the target analyte at the test line using environmentally friendly materials:
   i. Prepare a solution containing the target analyte or a suitable template molecule, functional monomers derived from renewable resources, green cross-linkers, and a green polymerization initiator.
   ii. Apply the solution to the test line region, followed by polymerization to form a highly specific binding cavity for the target analyte.
   iii. Remove the template molecule to generate a molecularly imprinted polymer (MIP) with selective binding sites for the target analyte.
d. Affinity-enhancing agents: Incorporate environmentally friendly affinity-enhancing agents at the test and control lines to improve the binding affinity and specificity of the capture molecules:
   i. Biocompatible molecular chaperones or engineered binding proteins that enhance the binding interactions between the capture molecules and the target analyte.
   ii. Biodegradable stimuli-responsive materials (e.g., thermoresponsive or pH-responsive polymers) that can modulate the local environment or conformation of the capture molecules, enhancing their binding affinity for the target analyte.
e. Advanced patterning techniques: Utilize advanced, eco-friendly printing or patterning techniques, such as inkjet printing with green functional inks or photolithography with biodegradable photoresists, to precisely immobilize multiple capture reagents with high spatial resolution. This enables multiplexed detection of various targets in a single assay.

Based on the above detailed description, LEIQA type test kits provide a substantially over legacy LFAs based on the use of environmentally friendly chemicals, eco-friendly signal amplification, molecular imprinting with sustainable materials, green affinity-enhancing agents, and advanced patterning techniques, to produce the test and control lines. These approaches significantly enhance the sensitivity, specificity, and utility of the LEIQA over traditional LFAs while minimizing environmental impact.

Lastly, for FIG. 5a, the test lines in one embodiment of LEIQA could be SARS-CoV-2 for 504, Influenza A for 506, Influenza B for 507, and Respiratory Syncytial Virus for 508.

In a legacy LFA, the control line serves as an internal quality control to ensure that the assay is functioning correctly and that enough liquid was used in the test. It is typically composed of secondary antibodies or other binding molecules that interact with the detection reagent, regardless of the presence or absence of the target analyte. Common antigen-antibody combinations used for the control lines in LFAs include:
a. Secondary antibodies: The control line often consists of species-specific secondary antibodies that are capable of binding to the detection reagent (which is usually an antibody conjugated to a reporter, such as a colored latex bead or gold nanoparticle). For example, if the detection reagent is a mouse-derived antibody, the control line will consist of anti-mouse secondary antibodies that capture the detection reagent as it migrates along the reaction membrane. The formation of a visible control line indicates that the detection reagent is functional and that the assay has been performed correctly.
b. Antibodies against the detection reagent: In some LFAs, the control line may consist of antibodies that specifically recognize and bind to the detection reagent (e.g., antibodies against a specific epitope on the detection antibody). This approach ensures that the control line forms only when the detection reagent is present and functional.
c. Streptavidin-biotin interactions: In some cases, the control line may utilize the high-affinity interaction between streptavidin and biotin. If the detection reagent is biotinylated, the control line can consist of immobilized streptavidin, which captures the biotinylated detection reagent as it moves along the membrane.

The choice of antigen-antibody combinations or other binding interactions for the control line depends on the specific design and requirements of the LFA. The primary goal is to provide an internal quality control that validates the functionality of the assay components and the proper execution of the test.

Control lines in LEIQA test kits provide several important advantages, various advanced chemical strategies and antigen-antibody pairs that can be employed individually or together to enhance the assay's performance, sensitivity, and utility over traditional LFAs, such as:
a. High-affinity engineered antibodies: Utilize engineered antibodies or antibody fragments (e.g., single-chain variable fragments, nanobodies, or aptamers) that demonstrate high affinity and specificity to target antigens. These engineered antibodies can potentially offer improved stability and ease of production while maintaining high-performance characteristics.
b. Multiplex detection: Incorporate multiple capture reagents for simultaneous detection of various targets in a single assay. This can be achieved by printing multiple test lines, each containing a specific antigen-antibody pair, or by using advanced patterning techniques to create distinct zones or regions on the reaction membrane. This enables comprehensive testing for multiple analytes or pathogens.
c. Environmentally friendly detection reagents: Replace traditional gold nanoparticles or colored latex beads with eco-friendly reporters, such as biodegradable polymer nanoparticles or quantum dots with low toxicity. These reporters can be conjugated to the detection antibodies to generate visible or quantifiable signals.
d. Advanced signal amplification: Incorporate environmentally friendly signal amplification strategies directly at the test and control lines to enhance the assay's sensitivity:
   i. Enzyme-linked antibodies: Conjugate eco-friendly enzymes, such as horseradish peroxidase (HRP) or alkaline phosphatase (AP), to the capture or detection antibodies, enabling enzyme-catalyzed amplification reactions at the test and control lines.
   ii. Green catalytic nanomaterials: Immobilize biodegradable or non-toxic catalytic nanomaterials, such as biodegradable polymer nanoparticles, at the test and control lines to amplify the signal generated by redox reactions or other catalytic processes.
e. Advanced capture strategies: Utilize innovative capture strategies to improve the specificity and sensitivity of the assay:
   i. Molecularly imprinted polymers (MIPs) can be used to create highly specific binding cavities for the target antigens, enhancing the assay's specificity and sensitivity while using environmentally friendly materials.
   ii. Biomimetic recognition elements, such as synthetic peptide ligands or molecularly engineered protein binders, can be used as alternatives to traditional antibodies, providing a more sustainable and eco-friendly approach.
f. Eco-friendly surface functionalization: Functionalize the reaction membrane with biodegradable or biocompatible surface chemistry to improve immobilization efficiency and orientation of capture molecules on the test and control lines.

FIG. 5b shows an actual picture of one embodiment of a LEIQA-AG where a stronger control line 519 is followed by test lines 520 521 522 and 523 of various intensity, and where the control and test lines all substantially are of the same color although varying in intensity. The “red” test and control lines in lateral flow assays (LFAs) are typically produced using colloidal gold nanoparticles, which serve as the most common visualizing agent in these tests. The red color is a result of the unique optical properties of gold nanoparticles and their localized surface plasmon resonance (LSPR) effect. The chemistry involved in producing the test and control lines typically starts with the conjugation of antibodies or antigens to the colloidal gold nanoparticles. These conjugated gold nanoparticles are deposited onto the conjugate pad of the LFA. When a sample is added to the sample pad, it rehydrates the conjugate pad and allows the gold nanoparticle-conjugated antibodies or antigens to mix with the sample. As the mixture migrates through the reaction membrane by capillary action, it encounters the test and control lines. The test line is coated with a specific antigen or antibody that is designed to bind with the target analyte (e.g., an antibody in the case of an antigen test or an antigen in the case of an antibody test) present in the sample. When the target analyte binds to the immobilized antigen or antibody on the test line, the gold nanoparticle-conjugated antibodies or antigens accumulate at the test line, producing a visible red color. The intensity of the color is proportional to the concentration of the target analyte in the sample.

In typical legacy LFAs, the control line is coated with a secondary antibody or a protein that binds to the conjugated antibodies or antigens on the gold nanoparticles. The control line serves as an internal quality control to confirm that the test is functioning correctly and that the sample has migrated adequately through the reaction membrane. When the gold nanoparticle-conjugated antibodies or antigens bind to the control line, a visible red color is produced, indicating that the test is working properly. This method of introducing secondary antibody and antigen pairs to produce the control line may cause cross reactivity with one or more of the test lines, a problem that exacerbates as more test lines are added to the same assay. One method of overcoming this, that this specification teaches, is to develop a control line conjugate that rather than being part of a specific antigen-antibody pair not part of the markers tested for, instead react to the other antibodies used to generate the test lines. A typical multi test done in the field will rarely show positive for all test markers, such as Covid, Influenza A, Influenza B and RSV all the same time and all with high intensity, thus there will always be some antibodies left for each test line that remains without a reaction to antigens in the sample. The invention here is to use the superfluous antibodies from the test lines as they wash through the reaction membrane to generate the control line 519, which means that the control line must be the line that is furthest away from the sample pad 515. The drawback with this method is that it will be hard for human eyes to differentiate between test lines and the control line because they are all the same color made from the same type of nanoparticles. This is overcome by using an electronic reader for the test kits, which also allows the various lines to be read quantitatively. Lastly, using the same nanoparticles for each test and control line improves quality control by not having to specify and verify a variety of nanoparticle types, which in turn increases procurement volume, contributing to an overall easier quality control and lower manufacturing cost, and because all markers use the same nanoparticles, quantification becomes easier as color consistency and calibration of concentration vs intensity is uniform.

These innovative chemical strategies, advanced antigen-antibody pairs, and eco-friendly materials can significantly enhance the performance, sensitivity, and utility of LEIQA test kit compared to a legacy LFA while minimizing environmental impact.

### LEIQA for Antibodies (AB)

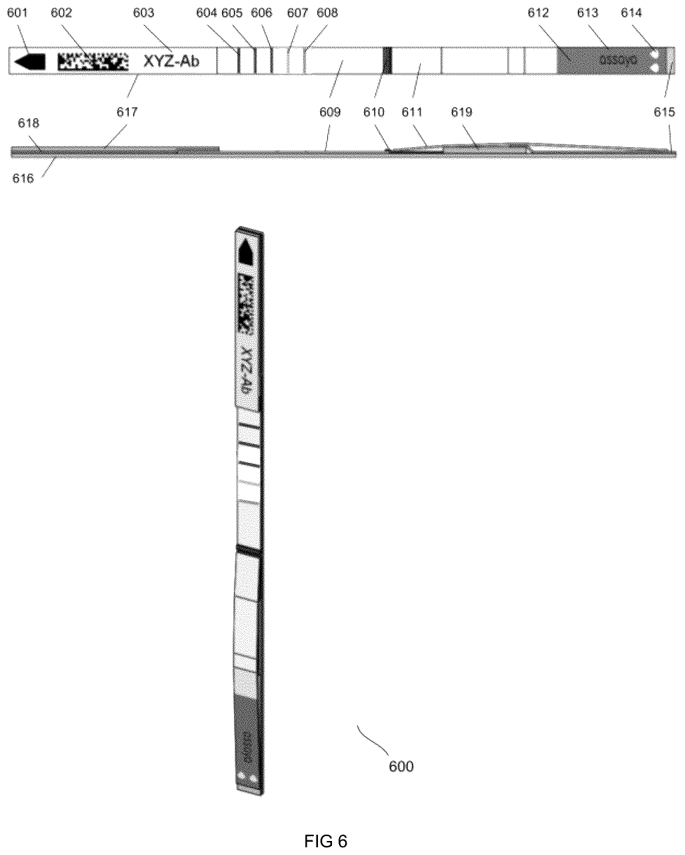

FIG. 6 shows a second type of LEIQAs “LEIQA-AB” 600 which is constructed to substantially detect AntiBodies (AB). Antibodies, also known as immunoglobulins, are proteins produced by the immune system in response to the presence of antigens. They play a crucial role in the body's defense against infections and other foreign substances. The various types of antibodies can also be used in diagnostic tests to detect the presence of specific antigens, or to determine if a person has been previously exposed to a particular pathogen or has developed immunity to it.

Immunoglobulins are proteins produced by B cells (a type of white blood cell) that play a vital role in the immune response. They are classified into five main classes or isotypes, each with distinct structure, function, and distribution. Here are the different types of immunoglobulins, along with their production sites and key functions:
a. Immunoglobulin G (IgG):
   i. Production: IgG is produced by plasma cells (mature B cells) in lymphoid tissues, such as the spleen, lymph nodes, and bone marrow.
   ii. Key functions: IgG is the most abundant immunoglobulin in the blood and plays a significant role in the immune response. It provides long-term immunity, neutralizes pathogens (such as viruses and bacteria), activates the complement system, and enhances the phagocytic activity of immune cells. IgG can also cross the placenta, providing passive immunity to the fetus.
b. Immunoglobulin A (IgA):
   i. Production: IgA is produced by plasma cells in the mucosal-associated lymphoid tissues (MALT), such as the gastrointestinal tract, respiratory tract, and genitourinary tract.
   ii. Key functions: IgA exists predominantly as secretory IgA (slgA) in external secretions like saliva, tears, and breast milk, as well as mucosal secretions of the respiratory, gastrointestinal, and genitourinary tracts. It plays a crucial role in mucosal immunity by neutralizing pathogens and toxins at mucosal surfaces, preventing their attachment to and invasion of epithelial cells.
   iii. Peyer's patches are small, organized lymphoid structures found in the mucosa-associated lymphoid tissue (MALT) of the small intestine, specifically in the ileum. These patches play a crucial role in the immune response against pathogens that enter the body via the gastrointestinal tract. The immunological mechanisms of Peyer's patches involve antigen sampling, processing, and presentation, as well as the induction of adaptive immune responses.
   iv. Antigen sampling: Peyer's patches contain specialized epithelial cells called microfold (M) cells, which are responsible for the transport of antigens from the intestinal lumen to the underlying lymphoid tissue. M cells efficiently take up particulate antigens, such as bacteria and viruses, through endocytosis, phagocytosis, or other transcytotic processes. After capturing the antigens, M cells deliver them to antigen-presenting cells (APCs), such as dendritic cells (DCs) and macrophages, within the Peyer's patches.
   v. Antigen processing and presentation: APCs, including DCs and macrophages, process the captured antigens and present antigenic peptides on their surface via major histocompatibility complex (MHC) molecules. DCs are particularly important in this process because they can present antigens to naïve T cells, initiating an adaptive immune response.
   vi. Lymphocyte activation and differentiation: Peyer's patches contain organized regions of B cells, T cells, and other immune cells. After antigen presentation, naïve T cells interact with the antigen-MHC complex on the surface of APCs, leading to their activation and differentiation into effector and memory T cells. B cells also encounter antigens in Peyer's patches, and with the help of T cells and other immune cells, they can become activated, differentiate into plasma cells, and produce specific antibodies against the antigens.
   vii. Immune response induction: Activated T and B cells can leave Peyer's patches and migrate to other parts of the body through the lymphatic system and bloodstream. Effector T cells contribute to the immune response by directly killing infected cells or by producing cytokines that help coordinate the immune response. Plasma cells secrete specific antibodies that neutralize pathogens and facilitate their clearance by other immune cells.
   viii. Secretory IgA production: In the context of the gastrointestinal tract, a critical component of the immune response is the production of secretory immunoglobulin A (IgA). Activated B cells in Peyer's patches differentiate into IgA-producing plasma cells, which then migrate to the lamina propria of the intestinal mucosa. Secretory IgA is released into the intestinal lumen, where it can neutralize pathogens and toxins, preventing their attachment to and invasion of the intestinal epithelium.
   ix. In summary, Peyer's patches serve as essential immunological sites in the small intestine, where they facilitate antigen sampling, processing, and presentation, as well as the induction of adaptive immune responses. These processes help protect the body from pathogens that enter via the gastrointestinal tract.
c. Immunoglobulin M (IgM):
   i. Production: IgM is produced by plasma cells in lymphoid tissues, such as the spleen, lymph nodes, and bone marrow.
   ii. Key functions: IgM is the first immunoglobulin produced during an immune response, offering the initial line of defense against pathogens. It is highly effective at activating the complement system and acts as an efficient agglutinating antibody, promoting the clumping of pathogens for easier clearance by phagocytic cells.
d. Immunoglobulin E (IgE):
   i. Production: IgE is produced by plasma cells in lymphoid tissues and is present at relatively low levels in the circulation.
   ii. Key functions: IgE plays a central role in allergic reactions and parasitic infections. It binds to specific receptors on mast cells and basophils, causing the release of inflammatory mediators (e.g., histamine) when triggered by allergens. Additionally, IgE contributes to the immune response against parasitic infections, particularly helminths (worms), by binding to and activating immune cells that target parasites.
e. Immunoglobulin D (IgD):
   i. Production: IgD is primarily found on the surface of immature B cells as a membrane-bound receptor, with small amounts present in the circulation.
   ii. Key functions: The exact function of IgD is not completely understood. However, it is believed to play a role in the early stages of B cell development and the regulation of B cell activation.

In summary, the different types of immunoglobulins (IgA, IgG, IgM, IgE, and IgD) are produced by plasma cells in various lymphoid tissues and have distinct functions in the immune response, including pathogen neutralization, complement activation, mucosal immunity, allergic reactions, and B cell development and regulation. LEIQAs are designed to be capable through the use of various collection mechanisms to detect all of the various types of these antibodies or immunoglobulins.

The LEIQA-AB 600 consists of a backing material 616 usually made from paper, cardboard or a paper/cardboard combination that has been treated to be hydrophobic through the application of a liquid-repellent substance as previously explained. Since LEIQA antibody tests are similar in construction to LEIQA antigen tests, this specification will only briefly list the elements of the LEIQA antibody version and only in detail teach the substantial differences.

A sample pad 615, similar in function to that used on legacy LFAs 411, and to a LEIQA-AG sample pad 515, but lengthwise longer and widthwise smaller to facilitate a slower reaction time, thus more sensitive as earlier described.

The sample pad 615 may be covered by a sheet 611, typically made from hydrophobically imprinted paper or cardboard, that comprises a “Specimen Collection Identification Color” (SCIC) 612 imprinted with a brand indicator 613, and a liquid drop icon 614 indicating which end of the assay should be placed into the buffer reagent liquid.

A main differentiating feature in a LEIQA-AB as compared to a LEIQA-AG is the presence of a filter 619, which could be a red blood cell (RBC) filter or a plasma separator between the sample pad and the conjugate pad. This filter plays an essential role in removing RBCs and other cellular components from the sample before it reaches the conjugate pad, ensuring optimal test performance, and prevents colorful parts of plasma or RBCs from entering the reaction membrane and thus interfere with the visual detection of chemical reactions that produce colored lines.

The RBC filter, often made of glass fiber or cellulose, works by taking advantage of the size and density differences between the cellular components and the liquid portion of the blood (plasma or serum). As the sample flows through the filter, the larger and denser cellular components, including RBCs, white blood cells, and platelets, are retained by the filter matrix. The plasma or serum, which contains the target antibodies or antigens, can then pass through the filter and reach the conjugate pad.

In some LEIQA antibody tests, the filter may also be designed to selectively retain specific blood components, such as IgM or other immunoglobulins, depending on the specific requirements of the assay. This selective retention can be achieved by modifying the filter matrix or incorporating specific capture reagents that bind to the target immunoglobulins.

By efficiently removing RBCs and other cellular components from the sample, the RBC filter ensures that the subsequent assay steps are not negatively affected by interference from these components. This filtering step is particularly important for antibody tests, as it helps to reduce background noise, improve the signal-to-noise ratio, and enhance the overall sensitivity and specificity of the test.

Compared to legacy LFAs, this filter part of the LEIQA may deploy several enhancements and improvements individually or in combination, such as but not limited to:
a. Size-based separation enhancement, where the RBC filter could be fabricated with advanced materials or engineered pore structures that provide more efficient size-based separation of plasma from cellular components. For example, nanoporous membranes or size-selective hydrogels that offer improved selectivity and filtration efficiency.
b. Surface modifications, where the filter surface could be modified with chemicals or biologicals to improve plasma separation or selectively retain specific components. For example, where the filter is coated with chemicals, such as polyethylene glycol (PEG), that reduce nonspecific binding and improve the flow of plasma through the filter.
c. Immobilized capture reagents, where the RBC filter is imprinted with specific capture reagents, such as antibodies, aptamers, or lectins, to selectively retain or remove specific components from the sample, which help enhance the assay's sensitivity and specificity by focusing on the target analyte while removing potential interfering substances.
d. Enzymatic treatments, where the RBC filter incorporates immobilized enzymes that selectively degrade specific blood components, such as proteases to break down unwanted proteins or glycosidases to remove specific carbohydrates, where this enzymatic treatment could help improve the flow of plasma through the filter and reduce potential interference from unwanted components.
e. Anticoagulant treatments, where the RBC filter is treated with anticoagulants, such as heparin or ethylenediaminetetraacetic acid (EDTA), to prevent blood clotting and improve sample flow through the filter, and where anticoagulant treatment is particularly beneficial when using whole blood samples or in situations where sample clotting might be an issue.

In summary, specific enhancements of the filter used in LEIQAs increases the performance over traditional LFAs in one or more ways in combination by specifically focusing on enhancing the RBC filter or plasma separator through size-based separation enhancement, surface modifications, immobilized capture reagents, enzymatic treatments, and/or anticoagulant treatments. These advancements lead to a more efficient separation of plasma from cellular components, improved sensitivity and specificity, and reduced interference from unwanted substances in the assay.

Moving now to the conjugate pad 610, and the reaction membrane 609, which operates similarly to the conjugate pad of a LEIQA-AG test, with the exception of that the combination of antigens and antibodies that react are reversed, such that the test indicates antibodies rather than antigens. The test line 604 is an example of an indicator that could for example indicate the presence of antibodies produced by the immune system in response to an infection with SARS-CoV-2 and specifically producing a reaction to the spike protein only found in those who have been vaccinated with mRNA or viral vector vaccines, due to the spike protein in SARS-CoV-2 vaccines being technically and biologically different from the spike protein found on the naturally occurring virus. Whether retrospectively effective or not, the modifications of the differences were introduced to enhance the safety, stability, and immunogenicity of the vaccines in these ways:
a. Prefusion stabilization: In many SARS-CoV-2 vaccines, the spike protein is engineered to be in its prefusion conformation, which is a more stable state than the postfusion conformation found in the actual virus during infection. The prefusion-stabilized spike protein is more effective at inducing neutralizing antibodies, as it exposes critical epitopes that are targeted by the immune system. To achieve this prefusion stabilization, researchers introduce specific mutations, such as the “2P” mutations (K986P and V987P), which lock the spike protein in its prefusion conformation and prevent the conformational changes required for membrane fusion.
b. Truncated versions: Some vaccines use only a part of the spike protein, such as the receptor-binding domain (RBD), which is responsible for binding to the ACE2 receptor on human cells. By focusing on this critical region, vaccines can induce a more targeted immune response against the virus. Using a truncated version of the spike protein can also reduce the chances of adverse reactions or immune responses targeting nonessential parts of the protein.
c. Codon optimization: In mRNA and viral vector-based vaccines, the genetic code for the spike protein is often optimized to enhance protein expression in human cells. This is achieved by altering the codons (triplets of nucleotides) in the genetic sequence to match the preferred codons in humans, without changing the amino acid sequence of the protein. Codon optimization can improve the efficiency of protein translation and increase the overall immunogenicity of the vaccine.
d. Additional modifications: Depending on the vaccine platform, additional modifications might be made to the spike protein or the way it is delivered. For example, adjuvants can be added to inactivated or subunit vaccines to enhance the immune response. In mRNA vaccines, the lipid nanoparticles used for delivery are designed to optimize the stability and uptake of the mRNA by cells.

In summary, the spike protein in SARS-CoV-2 vaccines is technically and biologically different from the spike protein found on the naturally occurring virus. These differences, including prefusion stabilization, the use of truncated versions, codon optimization, and additional modifications, are introduced to improve the safety, stability, and immunogenicity of the vaccine, ultimately leading to a different immune response from a natural infection, which can be used in the LEIQA-AB test to differentiate antibodies from vaccination vs natural infection.

The control line 605 functions similarly to the control line 505 for the AG-test except that the combination of antigen and antibodies may be switched.

A second test line 606 that may indicate the presence of antibodies formed against the nucleocapsid protein and that are only produced after a natural infection with SARS-CoV-2 and not as a result of vaccination because the currently authorized SARS-CoV-2 vaccines do not contain the nucleocapsid protein as their target antigen. Instead, these vaccines are designed to target the spike protein of the virus, which is responsible for mediating viral entry into host cells. The nucleocapsid protein is an internal structural protein of the virus that plays a crucial role in packaging the viral RNA genome and in the assembly and release of new virions. When a person is naturally infected with SARS-CoV-2, their immune system is exposed to the entire virus, including both the spike protein and the nucleocapsid protein. As a result, the immune system mounts a response against multiple viral proteins, generating antibodies against both the spike and nucleocapsid proteins, among others. However, the currently authorized vaccines (e.g., Pfizer-BioNTech, Moderna, and Johnson & Johnson) focus on the spike protein as primary target for antibodies. Since the vaccines do not contain or expose the immune system to the nucleocapsid protein, vaccinated individuals do not produce antibodies against this protein. This is why testing for nucleocapsid antibodies can be useful in determining whether a person has had a natural infection with SARS-CoV-2, as opposed to being vaccinated. It should however be appreciated that both antibodies (anti-spike and anti-nucleocapsid) described previously may be detected simultaneously in a human or animal that has been both vaccinated and naturally infected.

A third test line 607 and a fourth test line 608, etc. may be included on the LEIQA that indicates the presence of a third type antibody, such as COVID-19 (SARS-CoV-2) IgG/IgM, HIV 1/2, Hepatitis B surface antigen (HBsAg), Hepatitis C virus (HCV), Syphilis (Treponema pallidum), Dengue virus IgG/IgM, Zika virus IgG/IgM, Chikungunya virus IgG/IgM, Malaria (Plasmodium falciparum/Plasmodium vivax) antibodies, Lyme disease (Borrelia burgdorferi) IgG/IgM, Helicobacter pylori IgG/IgA/IgM, Influenza A/B virus IgG/IgM, Respiratory syncytial virus (RSV) IgG/IgM. It should be appreciated by those skilled in the art that the number of test lines available on a LEIQA is not limited to 5 as is shown here, and that the current version of the LEIQA specification currently defines up to seven test lines, but that this number may change as the demand for more tests per assay increases.

### Dual LEIQAs

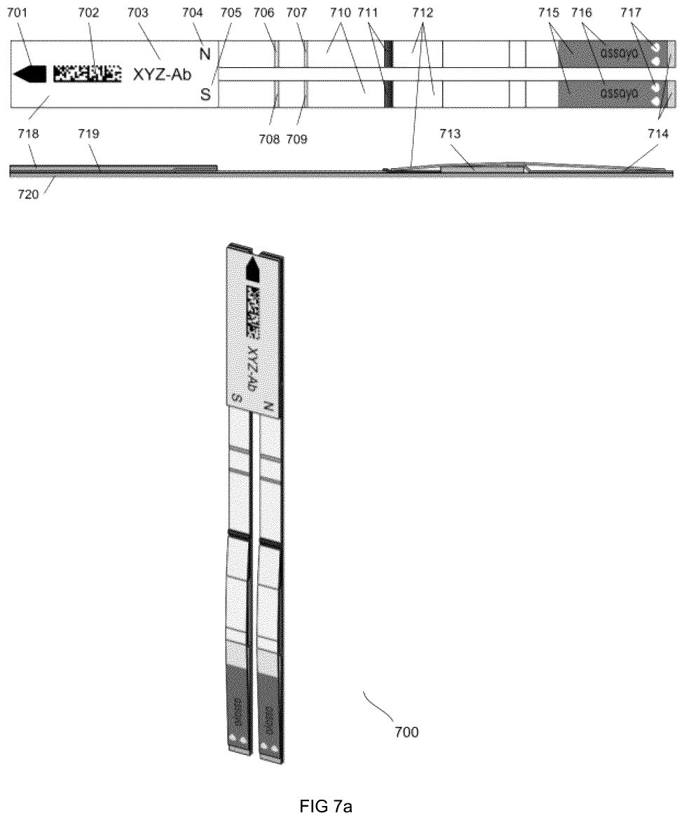

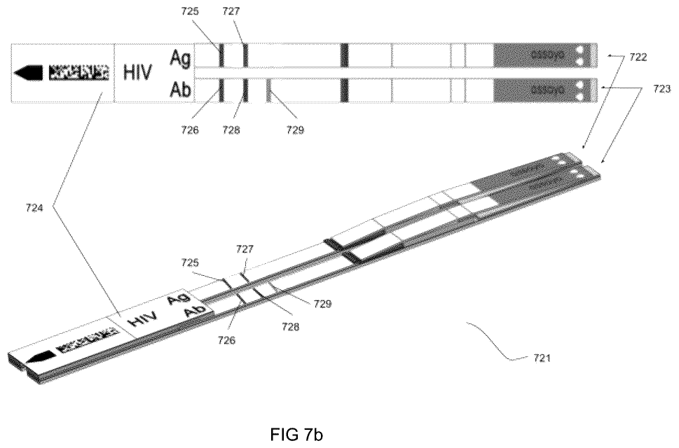

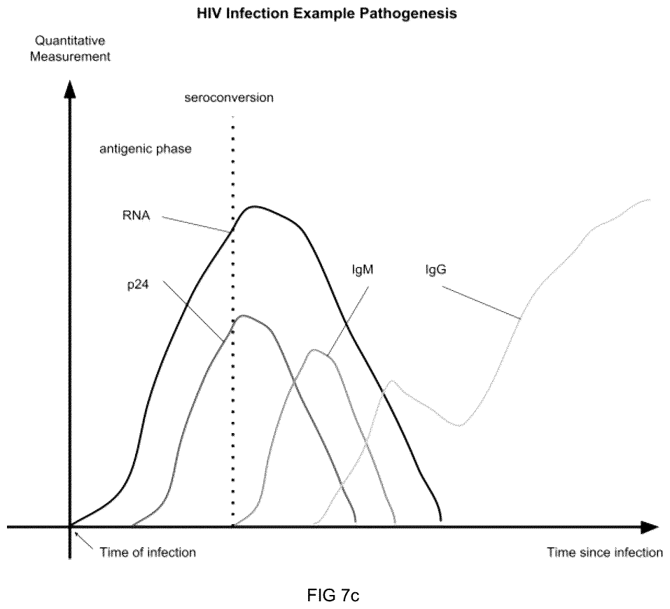

FIG. 7a shows a third type of LEIQA 700 which combines either two LEIQA-AG 500 devices, two LEIQA-AB 600 devices, or one LEIQA-AG 500 and one LEIQA-AB 600 device together. The devices are combined by affixing an adhesive sheet or label 718 across the multitude of devices, which holds them in place during manufacturing, packaging, shipping and use in the field, such as when being used to develop tests and being read by a machine. It should be appreciated that while this specification only teaches the combination of two devices, one can in a similar fashion extend this method to include three or more devices within the same product, which allows for even more tests to be performed from a single specimen or sample.

FIG. 7a further shows the label showing a QR-code, barcode, or DataMatrix 702, and a test name 703 along with specific indicators for each of the two LEIQAs within the dual LEIQA 704 indicating a first type of LEIQA and 705 indicating a second type of LEIQA. Similar to single LEIQA-AG and LEIQA-AB tests, with a multitude of test lines 706 and 708 and a multitude of control lines 707 and 709 spread across the two individual LEQIAs in a dual LEIQA. Furthermore, the dual LEIQA has the same features as for individual LEIQAs, such as conjugate pads 711, sample pads 714, sample pad covers 712, color coded specimen type indicators 715, brand indicators 716, liquid side or “wet-side” indicators 717, and one or more filters 713 depending on the test type.

Separating specific tests onto individual LEIQAs that are then combined into a Dual-LEIQA may also be advantageous for other important reasons that will now be described in detail.
a. One of the technical challenges associated with placing multiple test lines on the same Low Environmental Impact Quantitative Assay (LEIQA) is the potential for cross-reactivity between the various antibody/antigen conjugates. Cross-reactivity occurs when an antibody or antigen from one target reacts with an antigen or antibody of another target, respectively, leading to false positive or false negative results. In a multiplexed LEIQA, the presence of multiple test lines can increase the risk of such cross-reactions, as different antibodies or antigens may share structural similarities or binding sites, resulting in non-specific binding and inaccurate test results. Moreover, another limiting factor is the competition for binding sites among the target analytes. As the sample flows across the reaction membrane, multiple analytes may compete for the limited binding sites available on the antibodies or antigens immobilized on the test lines. This competition can affect the sensitivity and specificity of the assay, making it difficult to accurately detect and quantify the presence of each target analyte in the sample. In addition, placing multiple test lines in close proximity within a single LEIQA can also lead to issues with the flow of the sample and the conjugate along the reaction membrane. This can result in uneven flow rates, incomplete binding of the target analytes, and insufficient washing of unbound materials from the test lines. These factors can adversely affect the accuracy and reliability of the test results. Due to these technical challenges, it may be necessary to separate the multiple test lines into individual LEIQAs to ensure accurate detection and quantification of each target analyte. By employing separate LEIQAs, the risk of cross-reactivity and competition for binding sites is minimized, allowing for more accurate and reliable test results. Additionally, separating the test lines into individual LEIQAs enables better control over the flow rate and sample distribution across the reaction membrane, further enhancing the performance of the assay.
b. Another advantage of separating multiple test lines into individual LEIQAs is the ease of regulatory approvals. In situations where each of the individual tests has already undergone regulatory approval, employing separate LEIQAs can streamline the approval process for the new combined assay. By utilizing tests that have already been proven to be accurate, sensitive, and specific in their standalone format, the need for extensive cross-reactivity studies and additional validations can be significantly reduced. This can result in a faster and more efficient path to regulatory clearance, enabling the rapid deployment of the combined LEIQA test kit for diagnostic purposes. Additionally, by minimizing the need for extensive additional studies, cost and time investments in the approval process can be reduced, ultimately benefiting manufacturers, healthcare providers, and patients alike.
c. Another reason for separating multiple test lines into individual LEIQAs is the potential for improved sensitivity. When multiple test lines are combined in a single assay, the signal-to-noise ratio can be negatively affected, leading to reduced sensitivity and the possibility of false-negative results. By utilizing separate LEIQAs for each target analyte, the signal-to-noise ratio can be optimized, thus enhancing the sensitivity of the assay for each specific target. Furthermore, separating the test lines into individual LEIQAs allows for the optimization of each assay's specific buffer systems, conjugate concentrations, and reaction conditions. This tailored approach ensures that each test can perform at its highest level of sensitivity, providing more accurate and reliable results for each analyte being tested. In contrast, a multiplexed assay may require compromises in assay conditions to accommodate the presence of multiple test lines, which could negatively impact the overall sensitivity of the test. By employing separate LEIQAs, the challenges associated with multiplexing can be mitigated, resulting in improved sensitivity and a higher degree of confidence in the test results. This enhanced performance is particularly important in clinical settings where accurate detection and quantification of target analytes are crucial for effective diagnosis, disease management, and monitoring of patient outcomes.

Examples of dual LEIQAs can prove to be advantageous over individual tests are:
a. Dual antibody tests for antibodies against SARS-CoV-2, as described previously, that differentiate between vaccinal and natural antibodies, such as the SN-Ab test, where the S represents the spike antibody and the N represents the nucleocapsid protein antibody, can be used in combination to provide valuable insights into an individual's immune status. This information can help determine whether the observed immune response is due to vaccination or natural infection, guide appropriate public health interventions, inform clinical decision-making, and assess the need for booster vaccinations. Additionally, understanding the presence and levels of these specific antibodies can aid in evaluating the overall effectiveness of vaccination campaigns and contribute to ongoing efforts to control and mitigate the spread of the virus.
b. A HIV tests that combine multiple antibodies or combine antigens and antibodies, such as the dual LEIQA 721 illustrated in FIG. 7b, where one LEIQA device 722 and the other LEIQA device 723 and combined together and affixed through a common label 724, and where the first LEIQA is an Antigen (Ag) test that reacts to HIV antigens and where the second LEIQA is a dual Antibody (Ab) test that reacts to two types of antibodies.
c. The dual LEIQA illustrated in FIG. 7b, consisting of two separate LEIQAs designed for the simultaneous detection of HIV antigens and two distinct antibodies generated during different stages of the pathophysiological progression of the infection. In the first LEIQA, the assay is designed to specifically detect the HIV p24 antigen, which is a protein produced during the early stages of HIV infection with a testline 727 and a control line 725. This assay allows for the rapid identification of an acute HIV infection, as the p24 antigen can be detected in the blood within a few weeks of exposure to the virus, even before the appearance of detectable levels of HIV-specific antibodies, and where the second LEIQA is designed to detect two different antibodies, which are generated at different times during the progression of HIV infection. The first antibody, often referred to as a short-term antibody IgM, is produced in the initial phase of the immune response to the virus. This antibody can be detected within a few weeks to a few months following infection and is detected with the testline 728, and the second antibody, known as a long-term antibody (IgG), is generated during the later stages of the infection and can persist for years, providing a marker for long-term HIV exposure and being detected with the testline 729, and where a control line 728. By combining these two LEIQAs into a dual assay system, healthcare providers can obtain a more comprehensive understanding of a patient's HIV infection status. The simultaneous detection of HIV antigens and both short-term and long-term antibodies allows for the identification of acute, recent, and long-term infections, enabling more accurate diagnosis, timely initiation of antiretroviral therapy, and improved monitoring of the disease progression. FIG. 7c shows a typical progression of the types of stages of an infection that can be detected with the Dual LEIQA, and by reading the quantitative values for each of the tests, and converting the relative intensities of each test line to equivalence values, an assessment of where a particular patient is in the pathogenesis and infections cycle can be estimated.

### LEIQA for Chemical Markers Vs Biological Markers

Described earlier are LEIQA devices used for antigens (AG) and antibody (AB) detection, or a combination thereof. Both antigens and antibodies can be characterized as “biological markers”. Biological markers (or biomarkers) are substances derived from living organisms that can be measured in body fluids, cells, or tissues. They are usually molecules associated with a specific biological process or condition, and their presence, concentration, or change can provide information about a particular disease, physiological state, or response to treatment. Examples of biological markers detected by LFAs include:
a. Proteins: Antigens (e.g., viral or bacterial proteins) or antibodies (e.g., those produced in response to an infection or vaccine).
b. Nucleic acids: DNA or RNA fragments specific to a particular organism (e.g., SARS-CoV-2) or gene mutation.
c. Cells: Circulating tumor cells, white blood cells, or other cell types associated with a specific condition.

Chemical markers, on the other hand, are non-biological substances that can be detected in various samples, such as environmental samples, food products, or body fluids. They can be used to assess exposure to hazardous substances, contamination levels, or the presence of specific chemical compounds. Examples of chemical markers detected by LFAs include:
a. Small molecules: Drugs (e.g., opioids, amphetamines) or their metabolites, environmental toxins (e.g., pesticides, heavy metals), or other small organic or inorganic compounds.
b. Hormones: Chemical messengers (e.g., cortisol, human chorionic gonadotropin) that regulate various physiological processes in the body.
c. Enzymes: Specific enzymes associated with certain metabolic pathways or disease states (e.g., lactate dehydrogenase, creatine kinase).
d. Inorganic byproducts from biological processes such as oxygen from plants or carbon dioxide from eukaryotes.
e. Organic byproducts from biological processes such as Fibrin degradation products (FDPs), which are the byproducts formed during the breakdown of fibrin, a fibrous protein that plays a crucial role in blood clot formation. The process of fibrinolysis, which involves the dissolution of fibrin clots, generates FDPs. The biochemistry of fibrin degradation products involves several key components and enzymatic reactions.

This specification now describes one such marker, which can be considered to be both a chemical and a biological marker, and that would not typically be classified as an antibody or an antigen, Fibrin degradation products (FDPs).

FDPs are the byproducts formed during the breakdown of fibrin, a fibrous protein that plays a crucial role in blood clot formation. The process of fibrinolysis, which involves the dissolution of fibrin clots, generates FDPs. The biochemistry of fibrin degradation products involves several key components and enzymatic reactions.
a. Fibrin clot formation: When blood vessels are injured, a series of coagulation factors activate in a cascade, ultimately leading to the conversion of soluble fibrinogen into insoluble fibrin. Fibrin forms a mesh-like network that, along with platelets, creates a stable blood clot to prevent excessive bleeding.
b. Plasminogen activation: Plasminogen, a zymogen (inactive enzyme precursor), circulates in the blood. Upon activation, plasminogen is converted into plasmin, a proteolytic enzyme responsible for fibrinolysis. The conversion is mainly facilitated by tissue plasminogen activator (t-PA) and urokinase plasminogen activator (u-PA).
c. Fibrinolysis: Once activated, plasmin cleaves the fibrin mesh at specific peptide bonds, breaking it down into smaller fragments. These fragments are soluble and can be removed from the site of the clot.
d. Fibrin degradation products: The breakdown of fibrin by plasmin generates various-sized fibrin degradation products, including D-dimer, which is the smallest and most commonly measured FDP. D-dimer is a specific product formed when plasmin cleaves two cross-linked D domains of the fibrin molecule.

Elevated levels of FDPs, particularly D-dimer, in the blood can indicate active clot breakdown, suggesting the presence of thrombotic disorders such as deep vein thrombosis (DVT) or pulmonary embolism (PE). It is important to note that while elevated FDPs can signal an ongoing fibrinolytic process, they are not specific to any particular condition and should be interpreted in conjunction with clinical evaluation and other diagnostic tests.

D-dimer test is neither an antigen test nor an antibody test. It is a test that measures the levels of D-dimer proteins in the blood. D-dimer is a fibrin degradation product, which is produced when blood clots dissolve in the body. Elevated levels of D-dimer can indicate the presence of an active blood clot breakdown process, suggesting thrombotic disorders, such as deep vein thrombosis (DVT) or pulmonary embolism (PE).

The D-dimer test does not detect the presence of a specific antigen (e.g., a viral or bacterial protein) or antibody (e.g., an immune response to an infection or vaccine) in the blood. Instead, it measures the concentration of D-dimer proteins, which can provide information about the patient's blood clotting status and help healthcare professionals in the initial screening for thrombotic disorders or monitoring anticoagulant therapy.

A LEIQA is provided for the detection of D-dimer, an antigen indicative of blood clotting events. The LEIQA includes a sample pad for receiving a biological sample, a conjugate pad in fluid communication with the sample pad, a red blood cell (RBC) filter interposed between the sample pad and the conjugate pad, and a reaction membrane in fluid communication with the conjugate pad.

The RBC filter is configured to separate plasma from whole blood samples by trapping red blood cells, enabling the plasma containing the target D-dimer antigen to flow through the conjugate pad. The conjugate pad contains anti-D-dimer antibodies conjugated to signal-generating particles. The reaction membrane has a test line comprising immobilized anti-D-dimer antibodies and a control line comprising control antibodies.

The LEIQA is configured such that when a biological sample is applied to the sample pad, the plasma flows through the RBC filter and the conjugate pad, wherein the D-dimer antigen in the plasma binds to the anti-D-dimer antibodies conjugated to the signal-generating particles. The resulting complexes flow through the reaction membrane, binding to the immobilized anti-D-dimer antibodies on the test line and generating a visible signal proportional to the concentration of D-dimer in the sample. The control line captures excess conjugated antibodies, confirming proper fluid flow and assay functionality.

In some embodiments, the LEIQA may be used to detect elevated levels of D-dimer following a snake bite, particularly bites from snakes with venom that may cause coagulopathies or clotting disorders, such as certain species of vipers, pit vipers, or elapids. By measuring D-dimer levels using the LEIQA, healthcare providers can quickly determine the risk of clotting events or other blood clotting disorders associated with the snake venom, enabling timely and appropriate interventions to prevent complications and improve patient outcomes. The LEIQA thus provides a valuable tool for the rapid assessment of clotting risks following snake bites and can facilitate the management of snake bite-related coagulopathies.

The LEIQA disclosed herein may be further adapted for use in a variety of clinical settings and scenarios, providing timely and accurate quantitative measurements of D-dimer levels. The rapid detection and quantification of D-dimer levels can assist healthcare providers in the early diagnosis of various clotting disorders and the evaluation of the risk of thrombotic events, such as deep vein thrombosis (DVT), pulmonary embolism (PE), and disseminated intravascular coagulation (DIC).

The compact, portable, and user-friendly design of the LEIQA makes it suitable for use in a wide range of environments, including hospitals, clinics, and remote or resource-limited settings. The LEIQA requires minimal sample preparation and can deliver results within a short time frame, enabling clinicians to make informed decisions regarding the appropriate treatment course for patients presenting with elevated D-dimer levels or other clinical symptoms suggestive of clotting disorders.

Additionally, the LEIQA can be used for monitoring the effectiveness of anticoagulant therapy, guiding the management of patients with chronic clotting disorders, and evaluating the risk of recurrent thrombotic events. The ability to detect changes in D-dimer levels over time may also prove beneficial in the follow-up and management of patients with a history of vaccine-related injuries or adverse events, such as myocarditis, pericarditis, and micro thrombocytopenia.

In summary, the LEIQA for D-dimer detection provides a valuable tool for the rapid and quantitative assessment of clotting disorders and thrombotic events, with potential applications in a variety of clinical and public health scenarios. The disclosed LEIQA technology enables the early identification and management of patients at risk for clotting events, ultimately contributing to improved patient outcomes and the overall quality of healthcare.

Long COVID, also known as post-acute sequelae of SARS-CoV-2 infection (PASC), is characterized by a wide range of symptoms that persist or develop after the acute phase of a COVID-19 infection. The heterogeneous nature of long COVID makes it challenging to identify specific chemical or biological markers that can be used to quantify the condition universally. However, some potential markers have been proposed, which might be associated with long COVID symptoms or underlying mechanisms. These include:
a. Inflammatory markers: Persistent inflammation has been suggested as a possible contributor to long COVID symptoms. C-reactive protein (CRP), interleukin-6 (IL-6), and tumor necrosis factor-alpha (TNF-alpha) are some of the inflammatory markers that could be investigated in the context of long COVID.
b. Autoantibodies: Some studies have reported the presence of autoantibodies in long COVID patients, which might contribute to the development of various symptoms. Identifying specific autoantibodies and their target antigens could help understand the potential autoimmune aspects of long COVID.
c. Cellular immunity: The role of T cells and B cells in long COVID is still under investigation. Evaluating the persistence and functionality of SARS-CoV-2-specific T cells and B cells, as well as memory cell populations, could provide insight into the immunological aspects of long COVID.
d. Biomarkers of organ damage: Since long COVID affects multiple organ systems, evaluating organ-specific biomarkers, such as troponin for heart damage or D-dimer for blood clotting, may help assess the extent of organ involvement in patients with long COVID.
e. Viral load and persistence: Some studies have suggested that viral persistence or low-level viral replication might contribute to long COVID symptoms. In this context, detecting SARS-CoV-2 RNA or specific viral antigens in different body fluids or tissues could be investigated as potential markers for long COVID.

The LEIQA combining the chemical and biological markers described above is an inexpensive, easy to use, and rapid to develop and use tool that when combining multiple particular markers can be used for example to assess a person or animals present medical condition and past medical history, by the analysis of a multitude of markers in combination used a diagnostic device.

In order for these diagnostic test results to be useful, one must be able to measure the specific amplitude, signal or intensity of each of these markers, either instantly or by measuring and tracking them over time. A medical professional, a computer software program with algorithms, deep learning algorithms, the presentation of results to an artificial intelligence (AI) system, etc. can then be used to determine and diagnose one or more medical conditions.

A LEIQA that combines D-dimer and long COVID diagnostic tests from a patient's blood sample can provide valuable information on both blood clotting status and potential lingering effects of COVID-19 or post-vaccine complications. Elevated D-dimer levels can help identify patients with an increased risk of thrombotic events, which have been reported in some cases of COVID-19 infection and after vaccinations. By simultaneously detecting markers associated with long COVID, such as specific antibodies or inflammatory markers, this combined LFA can help clinicians better understand the interplay between clotting abnormalities and persistent symptoms. This comprehensive approach can facilitate early identification and management of patients experiencing post-COVID or post-vaccine complications, allowing for more targeted treatment strategies and potentially mitigating the long-term impact of these conditions on patients' health.

### SWAC, Spacket and LEIQA Combination

The spacket adhering to a table surface 200 helps in the practical use of the spacket, such that it is less likely to fall over during tearing off the top part of the spacket, and during insertion of the SWAC 400 as seen in FIG. 8a, and a SWAC and a LEIQA 500 together as seen in FIG. 8b. In FIGS. 8a and 8b, illustration 800 shows the application of first a SWAC 400 inserted into the spacket which releases the specimen collected into the liquid 802. Notice the liquid level is not substantially affected by the insertion of the SWAC. In FIG. 8b, a LEIQA-AB 500 is inserted, which after some seconds and minutes will reduce the liquid level 803 as the liquid is absorbed up and into the LEIQA.

In conclusion, the present invention discloses environmentally friendly, potentially polymer-free or near polymer free alternatives to traditional plastic test tubes, providing two embodiments, referred to as “spackets” (standing packets), that are primarily composed of commercially available, easily recyclable aluminum foil. These spackets incorporate a unique design allowing them to stand upright on a tabletop, which is crucial for their practical use. The first embodiment, the “double flap bottom” spacket, utilizes a rectangular aluminum sheet and a layer of sticky glue for adhering the spacket to a surface, ensuring stability during use. The second embodiment, the “concave bottom” spacket, features a rounded concave cup-like bottom surface, while still maintaining a secure seal through the application of heat and pressure on the aluminum sheets. Both embodiments effectively replace the traditional plastic test tubes, reducing environmental impact while preserving the essential functions and usability required for preparing samples for subsequent reactions with the specimen from SWACs and LFAs or LEIQAs.

5. Kit Packaging with Linking to Electronic Instructions for Use

The next part of the present invention relates to an innovative packaging solution for diagnostic test kits, such as aluminum pouches or packages (803), featuring a QR code (804) linking to electronic Instructions For Use (eIFU). Traditional test kits include printed paper IFUs in every bulk package, resulting in increased waste, weight, and difficulty in reading due to small print size and limited space for multiple languages. The eIFU system provides several advantages over conventional paper-based IFUs, including real-time updates, improved readability through zoom features, availability in multiple languages, and compatibility with automatic web translation tools. Users can download the eIFU as a PDF or print it on demand, reducing waste and improving accessibility.

FIG. 8c depicts a protective pouch (803) made from recyclable aluminum, featuring a QR code (804) that links to a webpage containing the eIFU. The pouch also displays a lot code (805), expiration date (806), eIFU number (807), company brand (808), and test type (809). A tear mark (810) indicates where to open the pouch, while a seam (811) seals the package during manufacturing after all necessary components have been placed inside.

FIG. 8d illustrates an example webpage accessed through the QR code (804) via URL (812), featuring the company brand (813), test kit overall type description (814), and an icon (815) indicating the test kit's machine-readability. A QR code (816) on this webpage links to a timer app as described earlier and shown in FIG. 2n and FIG. 2o. A language selector (817) allows users to change the eIFU language, while a kit reference number (818) is used for ordering and regulatory approvals lookup. An icon (819) and a textual description (820) of the assay type are displayed, in this case, a “nostril SWAC” collection device as described earlier and depicted in FIG. 1a. A video tutorial link (821) demonstrates how to use the test kit, while a print and PDF icon (822) enables users to download or print the eIFU.

### Legacy Lancets and Lancet Pens

A lancet is a small medical instrument used for making a tiny incision in the skin to obtain a small sample of blood for testing. Lancets are commonly used for glucose monitoring in people with diabetes or for other blood tests. The lancet itself is a small, sharp, pointed blade that is usually less than 3 millimeters in length.

A lancing device is a tool that holds the lancet and is used to puncture the skin to obtain a blood sample. It is also sometimes referred to as a lancet pen, lancing pen, or lancing device. The lancing device is designed to be used with a disposable lancet, which is inserted into the device and then used to puncture the skin.

It's worth noting that different manufacturers may use slightly different terminology to refer to the components of their lancet systems. However, in general, the lancet refers to the small, sharp blade used to puncture the skin, while the lancing device or pen is the tool that holds the lancet and allows for safe and easy blood collection.

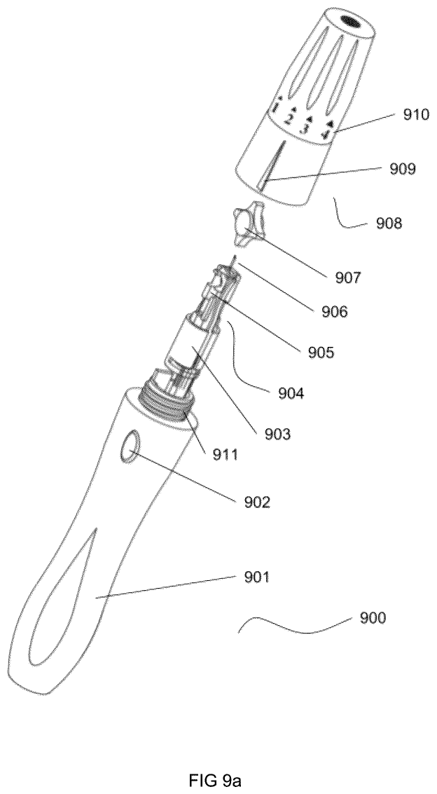

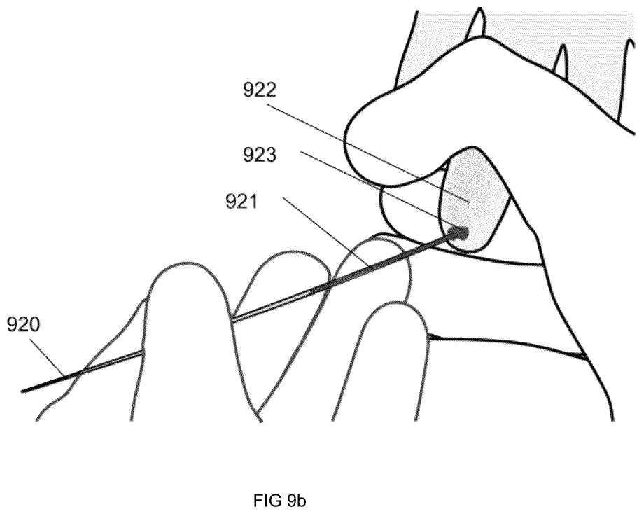

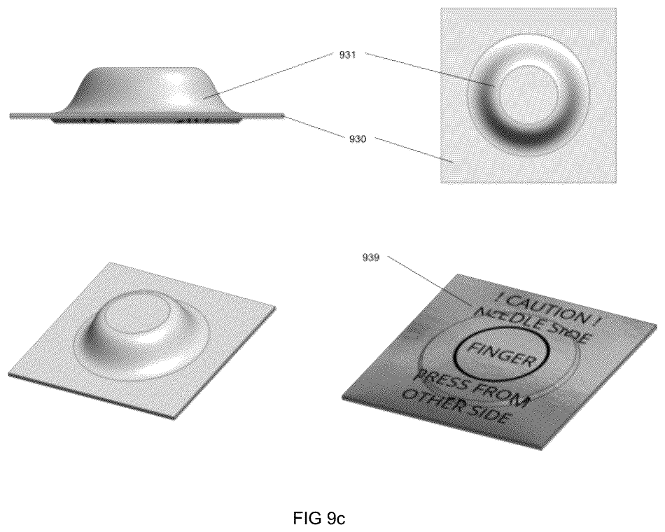

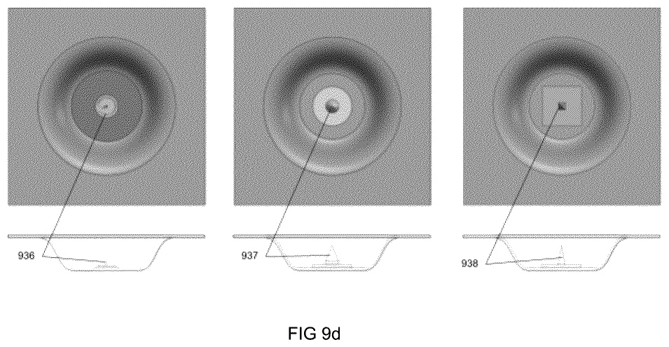

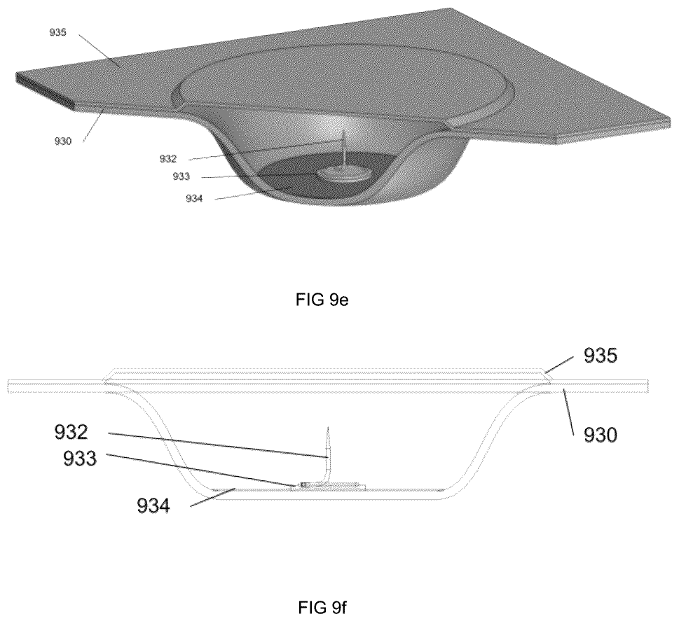

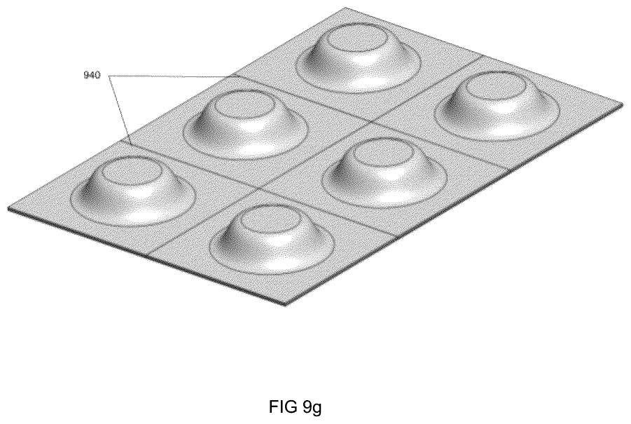

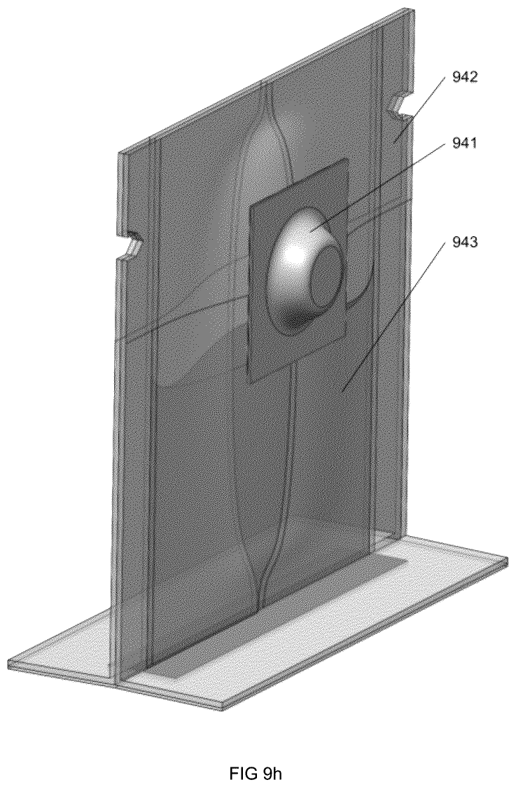

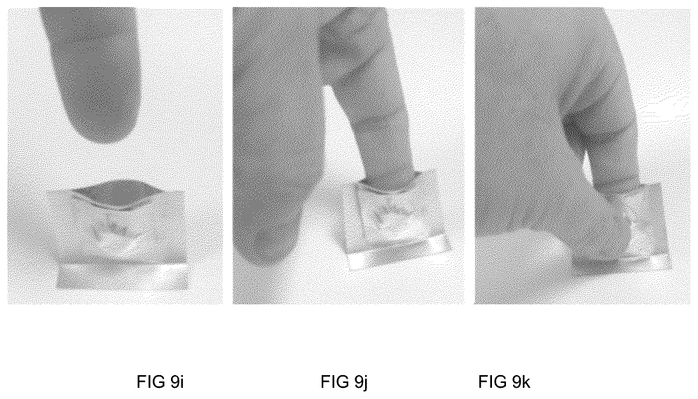

FIG. 9a shows one such prior art of a lancing device 900, which is available from a multitude of manufacturers in similar versions, where a pen like structure with a handle, also referred to as a “sliding barrel”, and a trigger button 902, a lancet carrier or lancet carrier tube 903, the actual consumable lancet device 904, with is typically comprised of a plastic injection body 905, the lancet needle 906, which is typically made from stainless steel, and a protective cap 907, which is affixed to the lancet body 904 to protect the needle 906 prior to use. A lancing device cover 908 consisting of a depth indicator dial 909, allows the operator to decide the depth of penetration from the needle but rotating the cover to a desired or suggested amount based on direction or experience. The depth of the penetration of the needle 906 into the skin is decided by the dial 910, which is generally configured by the dispense between the lancing device cover 908 and the lancing device body itself 900, which may be rotationally facilitated by the threads 911.

After a lancing device punctures the skin to obtain a blood sample, the capillary tube is held against the puncture site, and a small amount of blood is drawn up into the tube by capillary action. The capillary tube is then removed from the skin, and the collected blood can be used for various blood tests, such as glucose monitoring or cholesterol testing.

Lancing devices are often made of multiple materials, such as plastics, metals, and may contain electronic or mechanical components molded in as part of the assembly, which can make them difficult to recycle. The materials used in the device may need to be separated and processed individually, which can be time-consuming and expensive. Additionally, some components, such as the lancet holder, may be small or complex in shape, making them difficult to recycle through traditional methods.

The consumables used with lancing devices, such as lancets and capillary tubes, can also be difficult to recycle. Lancets are typically made of small, thin metal blades that are difficult to recycle due to their small size and shape. Additionally, lancets may be contaminated with blood or other bodily fluids, which can pose a health risk and require special handling and disposal procedures.

Overall, the complex and mixed materials used in lancing devices, along with the potential contamination of consumables, can make them difficult to recycle. While efforts are being made to develop more sustainable and environmentally-friendly options, such as reusable lancing devices or recyclable consumables, there is still a long way to go to ensure that these medical devices can be recycled in a safe and efficient manner.

### Legacy Capillary Tubes

Capillary tubes are designed to be single-use only, and are disposed of after they are used to collect a blood sample. They are commonly used in clinical settings, such as hospitals, clinics, and laboratories, and are also used in home-based blood glucose monitoring for people with diabetes.

FIG. 9b shows a typical application of one such capillary tube 920, where a tube is a small, narrow, and thin tube typically made of glass or plastic that is used to collect a small amount of blood 921 from a finger 922, causing a “finger prick wound” 923, after a lancing operation (for example by using the device from FIG. 9a). The capillary tube 920 is usually less than a few inches in length and has a very small diameter, typically around 1-2 millimeters.

Capillary tubes are typically made of glass or plastic, which technically can be recycled. However, they may be contaminated with blood or other bodily fluids, which can make them difficult to recycle through traditional methods. They may also be too small or too fragile to be processed through recycling machinery.

### 6. Sterile Blood Extractor (SBEX)

This specification now teaches a novel invention that replaces the function of a traditional lancet in one embodiment shown in FIG. 9c, FIG. 9d, FIG. 9e and FIG. 9f, where a sheet of aluminum 930 is formed to produce a dimple 931, in where a needle 932 is placed and affixed to the bottom center of the dimple by an adhesive 933, and where a liquid 834, such as ethanol may be added to keep the needle sterilized, and where a gas such as nitrogen may be also added to purge the volumetric chamber where the needle is from oxygen, thus preventing the needle from corroding or oxidizing. The volumetric chamber is enclosed by a secondary sheet of aluminum 935, and maybe be pressurized by adding a gas or by laminating the two aluminum sheets in a vacuum, which after the vacuum is released will cause the internal pressure within the volume to form a bubble that is pressurized. This pressure that causes both the dimple in the one aluminum sheet and the other aluminum sheet together to form this bubble, resists external pressure when pressed between fingers, rather than letting the aluminum to deform or collapse when pressed. The one aluminum sheet 935 being substantially weaker than the other 930 will cause the thinner weaker sheet 935 to suddenly and eventually rupture when enough pressure is applied by a finger, such that the finger breaks through the aluminum and hits the needle 932 causing the skin of the finger to be punctured and causing one or more drops of blood to ooze out of the small wound caused by the needle until it eventually coagulates and stops bleeding. The amount of blood that is released from a typical wound from this device can be substantially controlled by the size, form or shape and depth of the needle.

FIG. 9d shows different types of needles that can be used, for example a sharpened tip wireformed needle 936, a stamped out round needle 937, or a stamped out square or rectangular needle 938. Medical needles, such as those used for injections, blood collection, or suturing, are typically made from stainless steel. Stainless steel is the material of choice for medical needles due to its biocompatibility, strength, corrosion resistance, and ability to maintain a sharp edge. The most common type of stainless steel used in medical needles is the 300 series, particularly 304 and 316 stainless steel. Stainless steel needles are manufactured through various processes, including metal forming, cutting, and grinding, to achieve the desired shape, size, and sharpness. After manufacturing, the needles undergo sterilization, such as autoclaving, ethylene oxide gas sterilization, or gamma irradiation, to ensure they are safe for use in medical procedures. In some cases, specialized coatings may be applied to the needles to reduce friction and make the insertion less painful for the patient. These coatings can include silicone or other biocompatible materials that allow for smooth penetration through the skin and tissues. The needles used in this invention, maybe of course also use the traditional needle materials mentioned above, but because the needles used in this application is contained in a closed volume that may be purged of oxygen and sterilized by ethanol, they may also be manufactured from regular steel or aluminum, which significantly reduces cost and the ease of recycling, since both the needle and the combined can be recycled together without being separated.

Either aluminum sheets may be marked with text that explains how to use the product, and which side to press as shown in 939. FIG. 9g shows a typical mass production sample of this invention where perforated lines 940 allows sheets to be efficiently manufactured and where individual products can be torn off from a larger sheet.

As a product name for this invention we may use SBEX or SBEx 941 as an acronym that stands for “Sterile Blood Extractor”. FIG. 9h shows the SBEX combined with the formerly disclosed “spacket” 942 to form a product that replaces the need for both a traditional lancet pen 900 and needle insert 905 as well as replacing the need for the capillary tube 920 or pipette 407 used to collect and transfer blood to the reagent buffer solution 943 contained in the spacket. The operation of the SBEx combined with the spacket is shown in FIG. 9i, FIG. 9j and FIG. 9k, where in a first process the spacket top is torn off as seen in FIG. 9i, followed by a finger being inserted FIG. 9j and lastly the SBEX on the side of the spacket is pushed in FIG. 9k, which causes the needle inside the SBEX to puncture through the spacket and into the finger on the inside of the spacket such that blood drips into the buffer solution 943.

### 7. LEIQAPACK—Environmentally Friendly Packaging

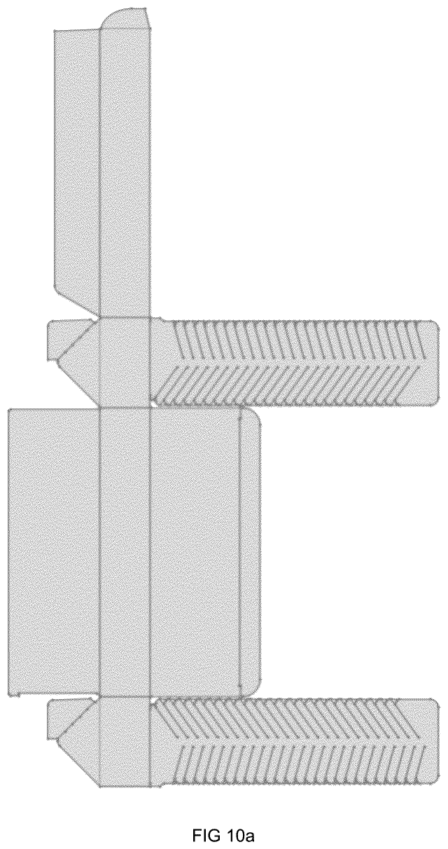

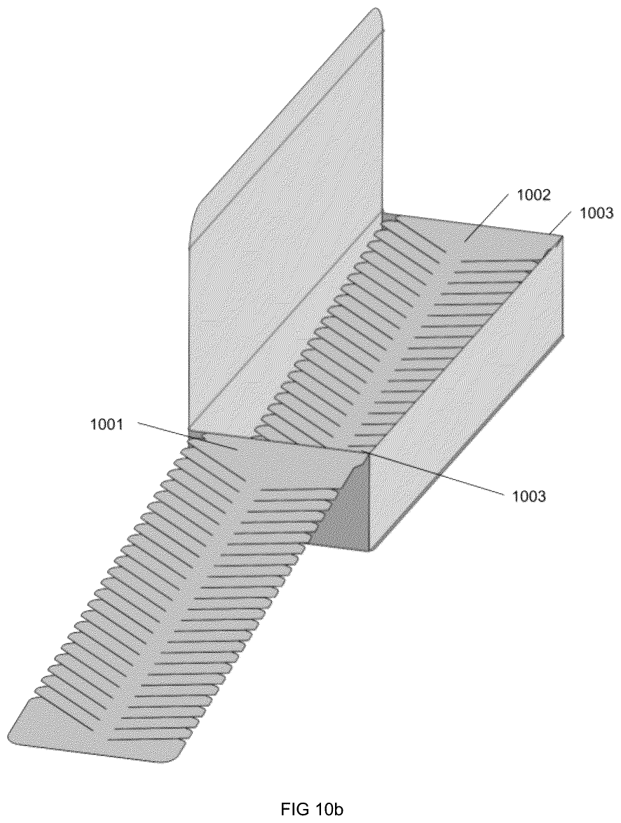

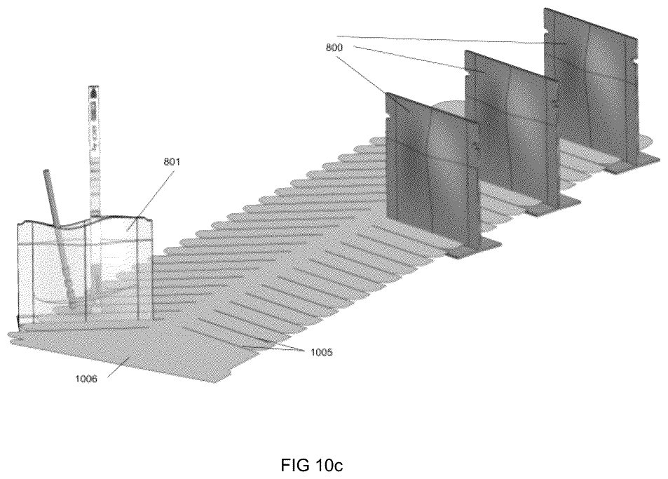

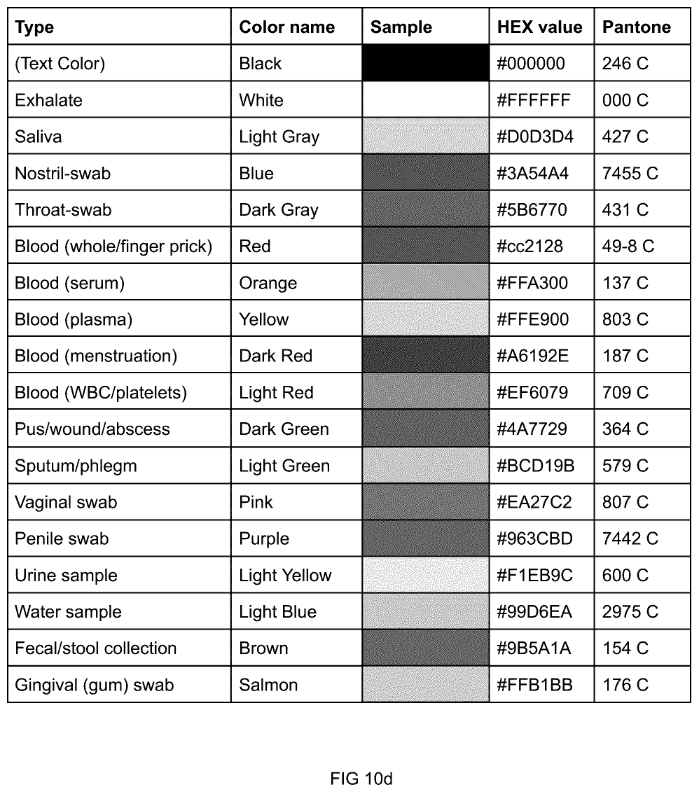

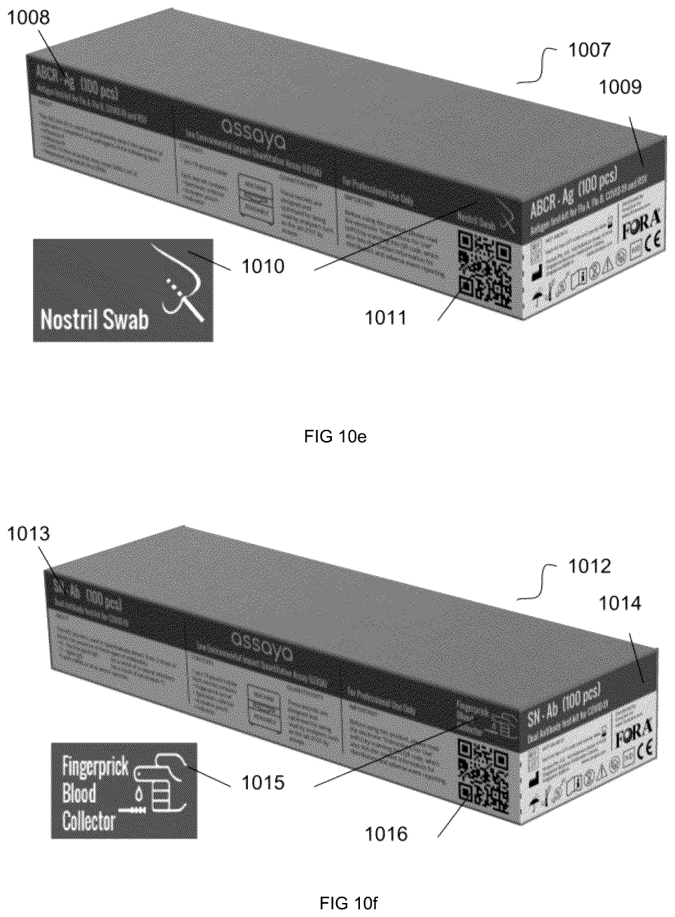

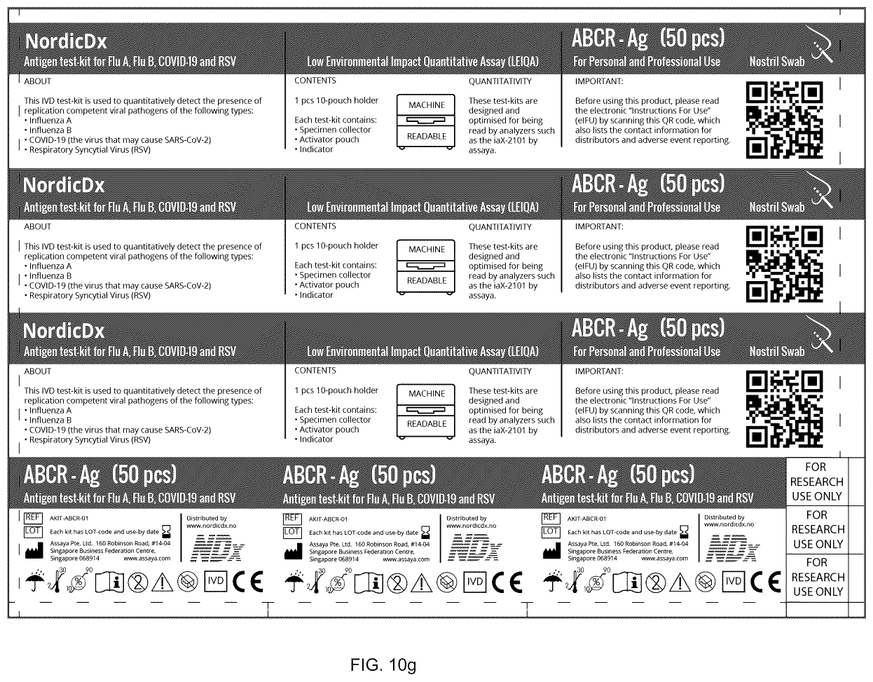

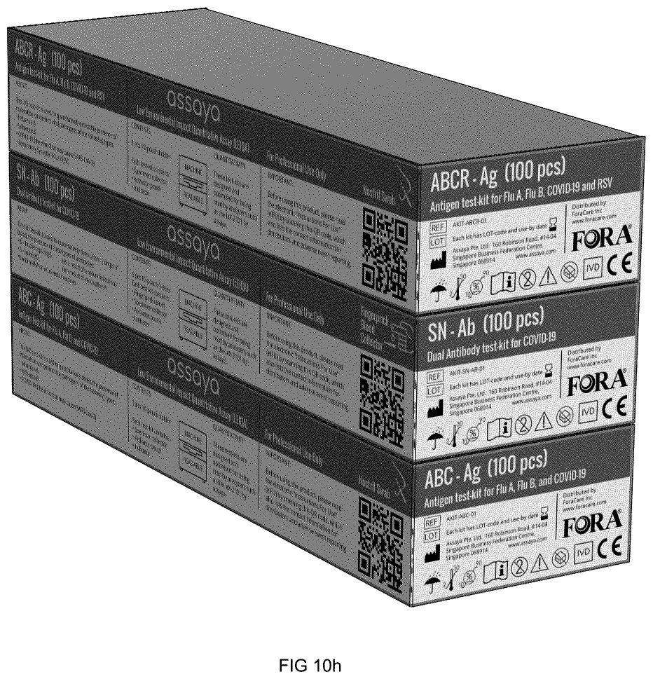

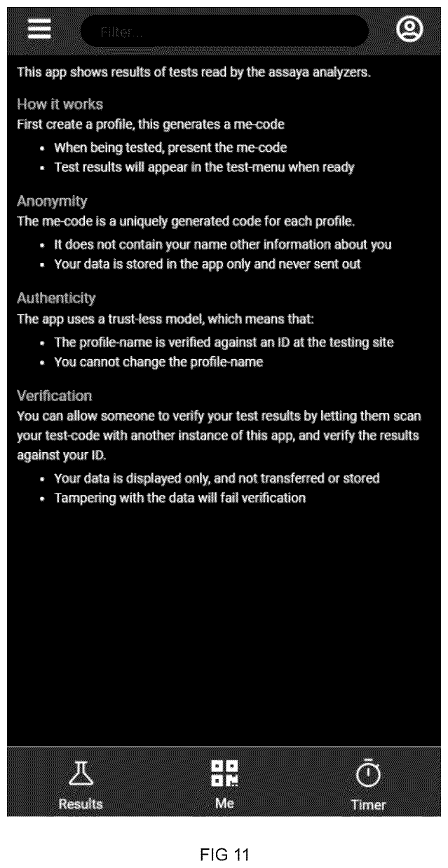

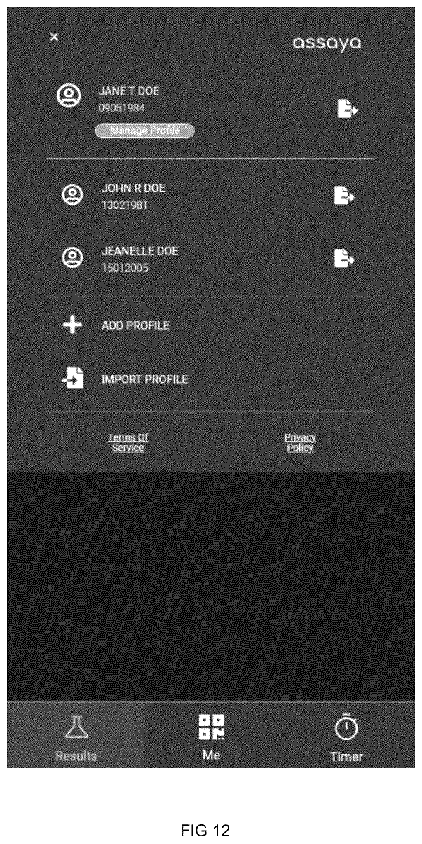

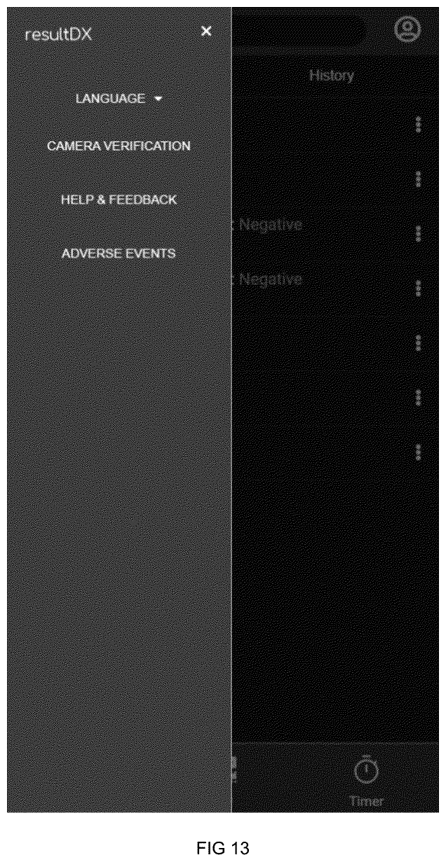

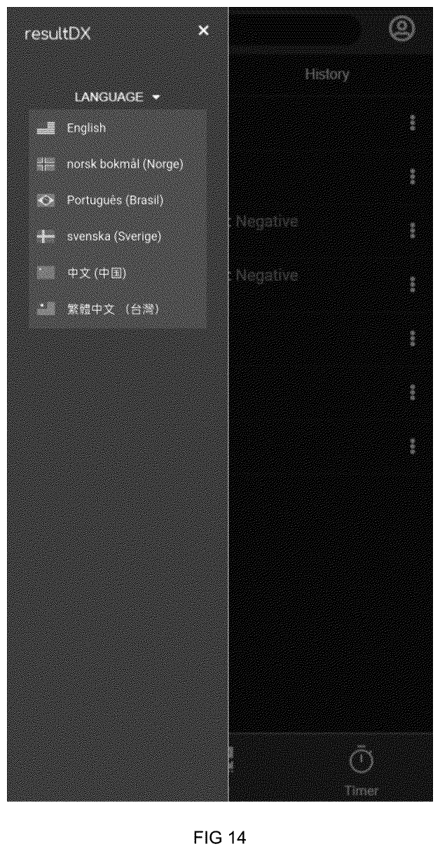

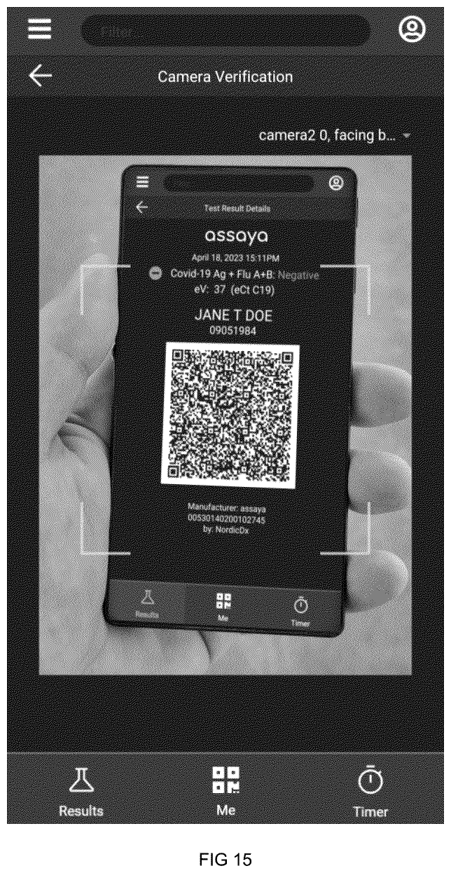

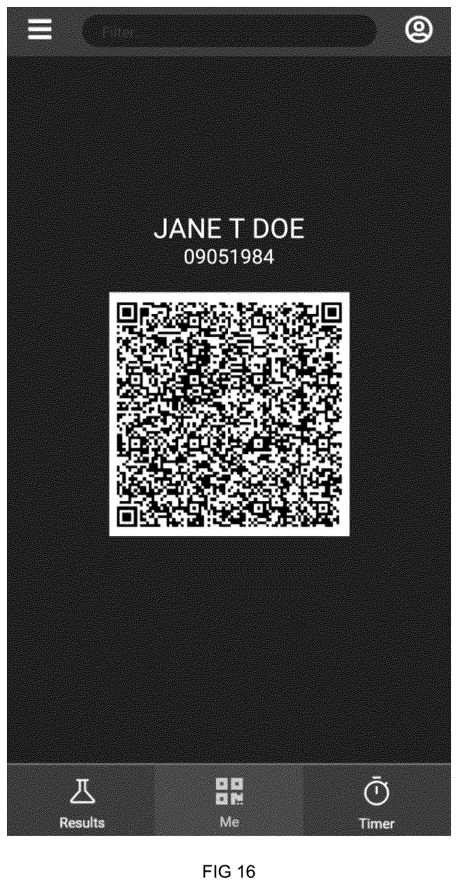

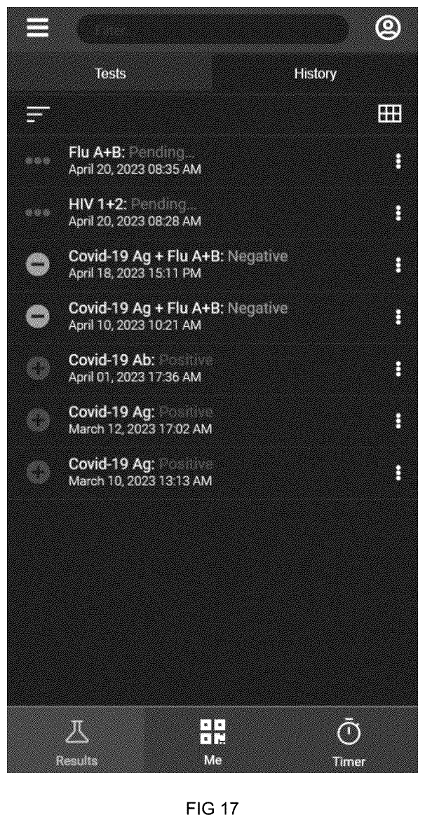

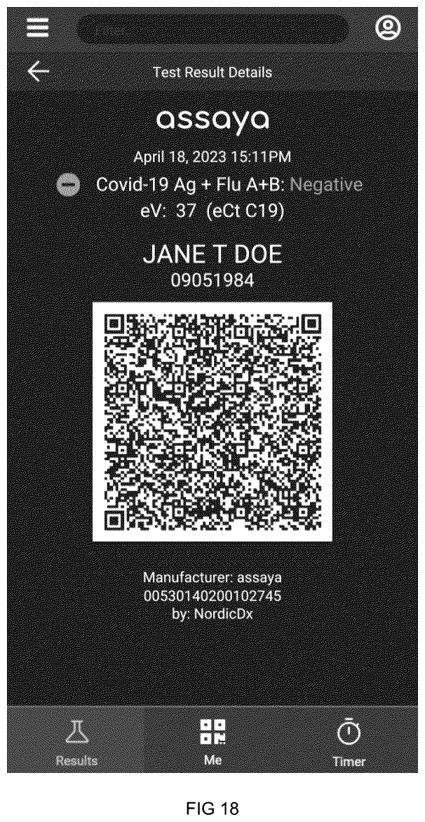

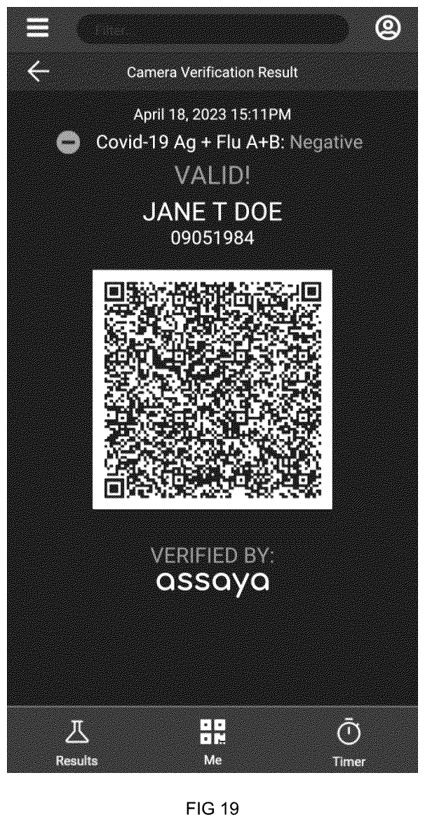

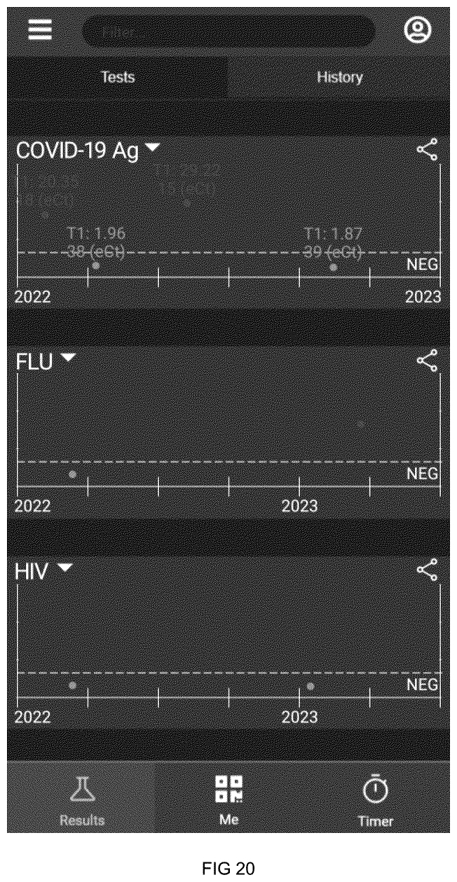

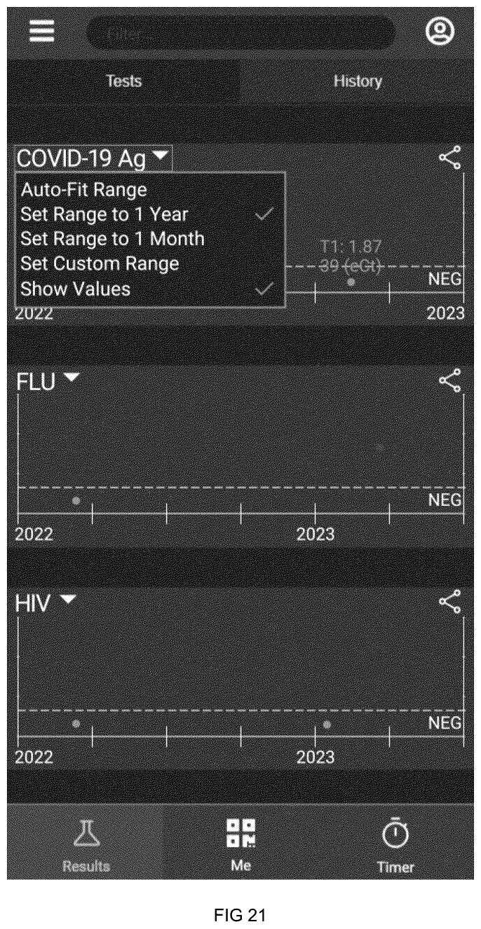

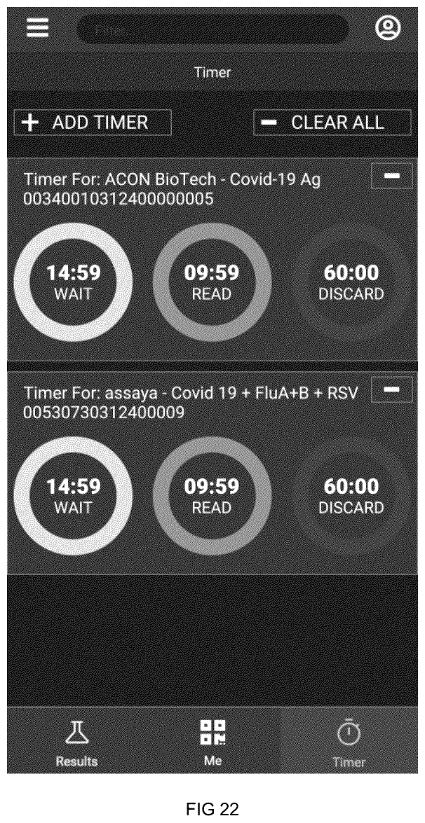

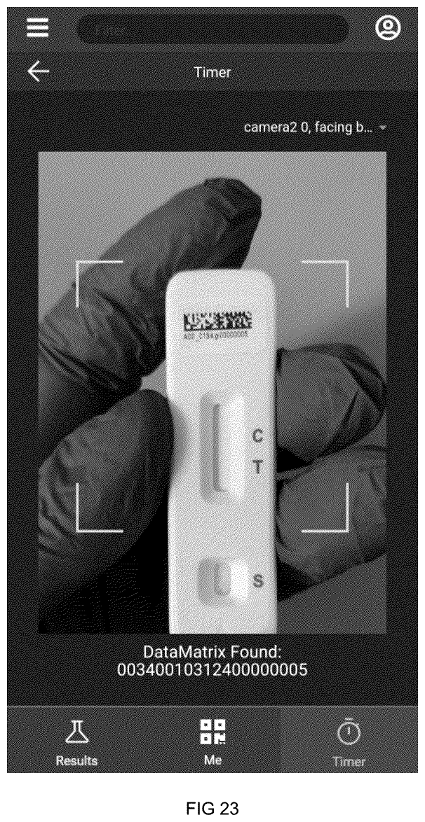

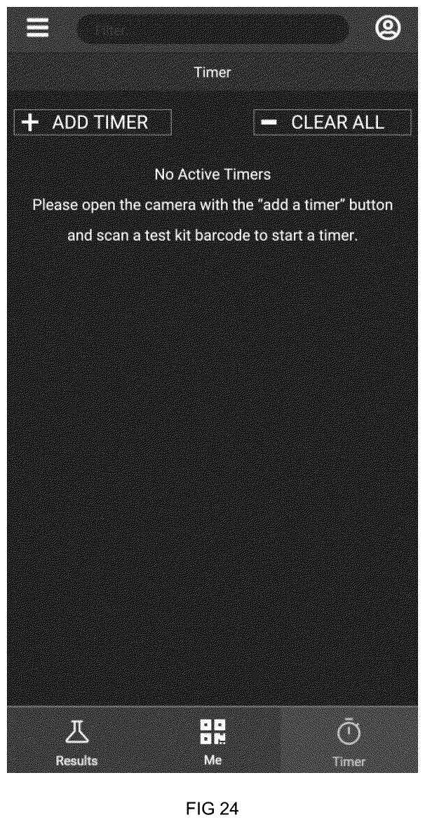

The next invention relates to the field of packaging for medical consumables, such as lateral flow assays (LFAs), and addresses the current lack of standardization in the packaging of such consumables. The absence of standardized packaging can result in increased costs, inefficient use of storage and shipping space, difficulty in handling, and potential confusion among healthcare professionals and end-users. In order to overcome these issues, the present invention proposes a packaging system that utilizes the standardized dimensions of a cigarette carton. The use of a uniform packaging size provides several advantages, including streamlined manufacturing, reduced packaging material waste, improved storage and transportation efficiency, and ease of handling for healthcare professionals and end-users. Furthermore, this standardized packaging system promotes consistency and familiarity across different medical consumables, thereby reducing the risk of errors and enhancing overall user experience. By incorporating the dimensions of a standard cigarette carton into the packaging design for LFAs and in particular LEIQA kits, the present invention provides a practical, efficient, and user-friendly solution to the challenges posed by the lack of packaging standardization in the medical consumables industry. Furthermore, using standard cigarette packaging allows pharmacies to use standard furniture traditionally used for storing and offering cigarette cartons for sale to be used for offering LEIQA test kits for sale, as is shown in FIG. 10h, where 3 LEIQA packs with 100 test kits each is placed on top of each other to create an unprecedented density of product while using existing standard packing materials, volume shipping materials and store furniture and fixtures.

In one embodiment of the invention, the packaging system features an innovative design that incorporates paper “ears” or “flaps” that are typically used to retain the lid of the box. In this embodiment, the paper ears or flaps are designed to be easily torn off and utilized as holders for “spackets,” small packets containing reagents or other consumables essential for the lateral flow assay or other medical tests. By integrating the spacket holders into the packaging itself, the need for additional components to hold and carry spackets is eliminated, resulting in reduced material waste and more efficient shipping. This design also simplifies the end-user experience, as healthcare professionals and users can readily access the spackets by repurposing the packaging components. Additionally, this embodiment contributes to the sustainability of the packaging system, as it minimizes the overall volume of materials used in the manufacturing and shipping processes, while still providing a functional and user-friendly solution.

FIG. 10a shows the flat pattern or cardboard cutout before the box is folded. FIG. 10b shows the box after it has been folded up and assembled, with two “ears” or flaps” 1001 and 1002, that can be torn off the box at semi-perforated lines 1003 and 1004. After these flaps have been torn off from the box, they can be placed on a table or flat surface. FIG. 10c shows one such flap placed on a table top, and where one or more spackets 800 slide into groves in the cardboard 1005, which are simple cut slots with rounded edges for ease of sliding the spackets into the flap.

Because of friction and that the spacket slightly bulges after the bottom due to its contents, the spackets with stay in the slid in position even if the entire flap carrier is lifted up, which gives this invention the additional advantage of being able to be used as a test kit carrier, which is useful for laboratory work, or to carry used products to where they need to get disposed off at. Note that this “flap carrier” 1006 additionally prevents the spacket from falling over during the operation of tearing off the top part of the spacket, during the insertion of a swab, a SWAC, and when inserting the LEIQA strips themselves.

### 8. Specimen Collection Type Color Coding and Iconography

This next part of the present invention introduces a novel color coding and iconography scheme to effectively and efficiently communicate the specimen collection method associated with various diagnostic test kits, particularly those incorporating Low Environmental Impact Quantitative Assays (LEIQAs). As the number and types of LEIQAs expand, healthcare workers and users may face difficulties in quickly and accurately determining the appropriate test kit usage method. The invention aims to address this issue by providing a clear, visual indication of the specimen collection method on the packaging of the test kits, streamlining the process and minimizing potential errors.

The invention, titled “Specimen Collection Type Color Coding and Iconography,” is presented as the eighth invention in the series of innovations described earlier. It comprises a color coding scheme, text label, and an icon that together indicate the specimen collection method required for each test kit. The color codes, icons, and text labels are applied to the packaging of the test kits, ensuring that users can easily and quickly identify the correct usage method.

FIG. 10d presents a table containing various specimen collection types, along with their corresponding color codes and hexadecimal color values. The table lists a diverse range of collection types, including nostril swabs, finger prick blood collection, saliva samples, and more, each represented by a unique color code.

FIG. 10e and 10f depict two different products utilizing LEIQAPACKs, recyclable cardboard boxes with outer dimensions similar to standard cigarette cartons. This packaging form factor is advantageous due to the widespread availability of inexpensive materials, innovative machinery, and large-scale processing technology for this specific size, as well as the efficient use of shipping pallets for bulk transportation.

In FIG. 10e, the main LEIQAPACK carton 1007 features a two-color label 1008 affixed to the outside. The label may consist of one wrap-around piece (1008 and 1009) or two separate labels for the front (1008) and side (1009). The top right corner of the box displays the text “Nostril Swab” and an icon representing the Sterile Wood Abrasive Collector (SWAC) used for nostril swabbing. This provides users with a clear and precise illustration of the intended usage method for the kit. Additionally, a QR code 1011 is included, linking to the electronic Instructions For Use (eIFU) described earlier.

Similarly, in FIG. 10f, the main LEIQAPACK carton 1012 has a two-color label 1013 affixed to the outside, which may also be a single wrap-around piece (1013 and 1014) or two separate labels for the front (1013) and side (1014). The top right corner of the box displays the text “Fingerprick Blood Collector” and an icon representing the Sterile Wood Abrasive Collector (SWAC) used for finger prick blood collection and subsequent transfer into the spacket. This offers users a clear and precise illustration of the correct usage method for the kit. A QR code 1016 is also present, linking to the electronic Instructions For Use (eIFU).

FIG. 10g demonstrates how a complete label set for three LEIQAPACKs can be printed on a standard A4 sheet by separating the two labels, enabling efficient label production. After the sheet has been printed, typically on paper with adhesive backing, and on paper that has been treated to avoid smudging from moisture or droplets, the sheet can be cut with a stamping tool to produce the 3 sets of individual labels.

Overall, the “Specimen Collection Type Color Coding and Iconography” invention provides a highly effective means of conveying essential information regarding the appropriate usage method for various diagnostic test kits, enhancing clarity and minimizing potential errors in specimen collection.

9. Smart Phone App with Multi-Timer

This next invention can be called “Secure Cloud-based Medical Diagnostic Test Data Management and Sharing System”, and relates generally to medical diagnostic test data management, and more specifically, to a secure cloud-based system for storing, accessing, and sharing medical diagnostic test data through a smart device app while preserving user privacy.

Medical diagnostic tests, such as Low Environmental Impact Assays (LEIQAs) and Lateral Flow Assays (LFAs), are widely used for diagnosing various medical conditions. The results of these tests are critical for healthcare providers and patients to make informed decisions about treatment and care. However, current systems for managing and sharing diagnostic test data often require users to disclose personal information, which raises privacy concerns and may deter some individuals from using these systems.

Furthermore, existing systems may not provide efficient means for sharing medical diagnostic test data between patients, healthcare providers, and other authorized individuals, such as family members or caregivers. This can result in delays in accessing test results and potential miscommunication between parties involved in patient care.

The present invention addresses the above-mentioned problems by providing a secure cloud-based system for managing and sharing medical diagnostic test data while preserving user privacy. The system utilizes a smart device app to generate unique identifiers called “me-codes” for each user profile, which are linked to personal data inputs but do not contain any discernible personal information. Users can create multiple profiles, share them with others, and access test results in real-time without disclosing their personal information.

The invention also allows healthcare providers to verify the authenticity of medical diagnostic tests by scanning unique DataMatrix codes on test kits and me-codes provided by patients. Additionally, the app includes features for collecting symptoms and vital sign data, visualizing test results graphically, and timing the development and validity of diagnostic tests.

We will now describe the invention in detail. In the following detailed description, reference is made to the accompanying drawings, which form a part hereof, and in which are shown by way of illustration specific embodiments in which the invention may be practiced. These embodiments are described in sufficient detail to enable those skilled in the art to practice the invention, and it is to be understood that other embodiments may be utilized, and that structural, logical, and electrical changes may be made without departing from the spirit and scope of the present invention.

The system comprises a smart device app that connects to a web application portal called assayaDX through an API called resultsDX. The app can be run as a progressive web application or as an app on iPhone or Google Play app stores. The backend system involves a processor in a cloud computing data center, such as AWS, Azure, or Google Compute Cloud (GCP), which runs an API handler to process requests from the app and access stored medical diagnostic test results from the cloud database.

Users can create one or more profiles within the app, which may contain personal information. However, this personal information is not transmitted to or stored in the cloud database. Instead, the profiles generate unique identifiers called “me-codes” based on the date and time the profile is created and the user's personal data inputs. The personal data undergoes a non-symmetrical hashing algorithm and data scrambling process, resulting in a me-code that is linked to the data input without containing any discernible data.

The app allows users to share profiles with others by exporting an encrypted string as text with a passcode, which can be sent via text message, email, private message, or stored as a text file. The recipient can then use the passcode to unlock the code and open a duplicate profile on their device.

Medical diagnostic test data is generated through the use of LEIQA or LFA tests, typically performed in a point-of-care healthcare setting. Patients present their me-code to healthcare workers, who use an LFA reader (e.g., iaX-2101 from assaya) to scan the unique DataMatrix code on the test kit and me-code provided by the patient. The test data and me-code are combined and stored in the cloud database.

The app also prompts users to enter symptoms, vital signs, and vaccination data, which are stored in a metadata section of a data packet transmitted to the cloud database with the me-code as reference.

Test results can be viewed in the app in list form or as graphical presentations with quantitative results. The app also includes a timer feature for managing test development and validity times, with an option for in-app purchase to enable multiple parallel timers.

### 10. Spacket Carrier and Multi Timer Sheet

The present invention relates to a water-resistant cardboard sheet 2500 designed to simplify the process of tracking and handling multiple diagnostic assays, particularly LEIQA or LFA tests, in laboratories, hospitals, and mass testing facilities. The sheet serves as a convenient spacket carrier, preventing spackets from spilling or falling over during use. It comprises multiple sections (10 shown, but any number is possible), the first slot a slot number text indicator 2501, a QR code 2502, and a rectangular groove 2530 for sliding in a spacket 2504. The QR code resolves to a URL containing parameters such as development time, test result validity time, and slot number, e.g., “https://assaya.com/timerdx/?time=600&valid=300&slot=1”. When scanned with a smart device, the URL directs users to a web page application that keeps track of individual timers for each section and its corresponding spacket, reducing the risk of confusion, mixup, and human error often associated with traditional laboratory timing devices.

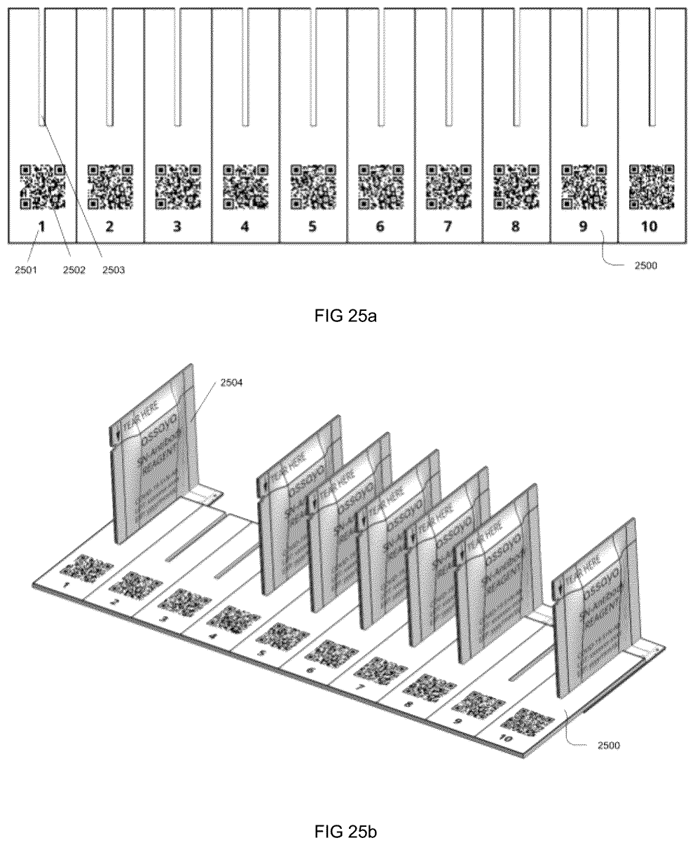

FIG. 25a shows a top view of the water-resistant cardboard sheet 2500 separated into 10 sections, although any number of sections may be used in other embodiments. Each section features a slot number 2501 for identification, a QR code 2502 for accessing the timer web application, and a rectangular groove 2530 designed for sliding in a spacket 2504, facilitating secure carrying and preventing spillage or falling over during use.

FIG. 25b provides an isometric view of the cardboard sheet from FIG. 25a, illustrating the insertion of spackets 2504 into some of the slots, while other slots remain unpopulated for clarity.

### 11. Complete Improved Diagnostic Test Solution

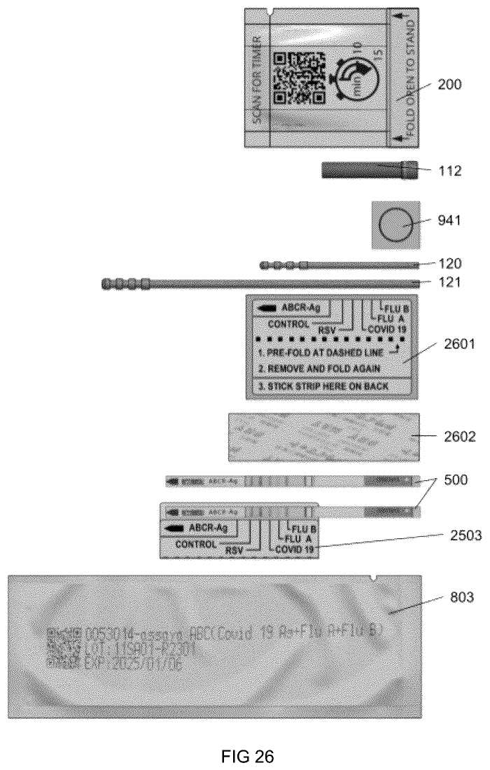

FIG. 26 shows a complete summary overview of all the various components disclosed in this specification, starting with the spacket 200, the PEC—Pathogen Exhalate Collector 112, a Sterile Blood Extractor (SBEx) 941, a Sterile Wood Abrasive Collector (SWAC) for human orifices 120 and for larger animal orifices 121, a printed sticker or label 2601 that shows the position of the various control and test lines. The sticker may be folded and placed on a dry-mat or dry-pad 2602 to produce a shape similar to a traditional plastic test cassette, or simply folded such that part of the adhesive of the sticker adheres to the actual LEIQA 500 shown in 2503. Lastly the pouch 803 is shown that contains or may contain some or all of the items listed above, depending on the specific configuration of the test kit.

1-8. (canceled)

9. A Sterile Wood Abrasive Collector (SWAC) for collecting specimens from one or more of: nostril, nasal cavity, mouth, ears, genital and rectal areas, for diagnostic testing, the SWAC comprising:
a single-piece construction of a biodegradable, renewable material with an integrated cylindrical handle and a collector head; and
the collector head comprising:
a series of alternating larger and smaller cylinders; and
a rounded front at a tip of the collector head.

10. The Sterile Wood Abrasive Collector (SWAC) of claim 9, wherein natural hydrophilicity, surface roughness, swelling properties, and enhanced detergent interaction due to natural properties of the biodegradable, renewable material facilitate release of a collected specimen from the collector head when the SWAC is contacted with a reagent buffer containing a detergent.

11. The SWAC of claim 9, wherein the biodegradable, renewable material is bamboo.

12. A method of releasing a collected specimen of a biological tissue sample from a Sterile Wood Abrasive Collector (SWAC), comprising:
contacting a collector head of the SWAC with a reagent buffer containing a detergent;
allowing detergent molecules in the reagent buffer to interact with the collector head thereby causing fibers of the SWAC to swell upon immersion in the reagent buffer, causing deformation of the fibers; and
as a result of a dual interaction between hydrophilic regions of the detergent and hydroxyl groups present on the collector head, as well as hydrophobic regions of the detergent, and the biological tissue sample to solubilize the biological tissue sample and effectively release the biological tissue sample from the fibers.

13-14. (canceled)

15. A method of making a molded swab to collect a biological tissue sample, the method comprising:
filtering and grinding wood particles and sawdust;
mixing the wood particles with a biodegradable adhesive to create a homogeneous mixture;
molding the homogeneous mixture in a mold including a handle and a collector head, the collector head comprising a plurality of alternating sized cylinders to form a swab;
applying heat and pressure to the mold to sterilize the swab and cure the adhesive; and
sanding the swab.

16. A nasopharyngeal swab comprising:
a single-piece construction comprising a handle and a collector head made of a natural material;
the collector head comprising a plurality of cylinders that create surfaces for specimen collection.

17. The swab of claim 16, further comprising:
wherein the collector head is cylindrical, and the collector head comprises alternating smaller and larger cylinders, where the larger cylinders scrape off epithelial cells and the smaller cylinders collect and retain the scraped epithelial cells.

18. The swab of claim 16, further comprising a rounded front to reduce resistance.

19. The swab of claim 16, wherein the natural material comprises one of: bamboo, wood, and molded wood particles with a biodegradable adhesive.

20. The swab of claim 19, wherein the biodegradable adhesive comprises one of: starch-based glue, lignin-based binder, and protein-based adhesive.

21. The swab of claim 16, wherein the collector head releases a specimen upon insertion into a liquid buffer.

22. The swab of claim 21, wherein the natural material is hydrophilic, and an absorption of water releases epithelial cells for a nasal swab.

23. The swab of claim 16, wherein the swab is sterilized using gamma irradiation.

24. A method of manufacturing a swab comprising:
receiving a piece of a natural material;
machining the piece to create cylindrical cuts in a first portion of the piece to form a collector head, and retaining a second portion of the piece without cuts to form a handle, the collector head and handle together comprising the swab;
sterilizing the swab; and
packaging the swab for use.

25. The method of claim 24, wherein the sterilizing comprises:
applying gamma irradiation to the swab.

26. The method of claim 24, wherein the sterilization comprises:
sterilizing the swab with ethylene oxide.

27. The method of claim 24, further comprising:
sanding and smoothing the swab, prior to the sterilizing.

28. A one-piece swab for collecting biological material specimen from an orifice for diagnostic testing, the swab comprising:
a handle portion for controlling the swab; and
a collector head for insertion into the orifice, the collector head comprising a cylindrical shape with a plurality of grooves cut into the cylindrical shape, the grooves to collect the biological material specimen from the orifice;
wherein a material of the swab is hydrophilic, such that the biological material is released from the grooves when the swab is inserted into a liquid buffer.

29. The swab of claim 28, wherein the swab is designed to collect one or more of: epithelial cells from a nasal cavity, gingival crevicular fluid (GCF) from a mouth.

30. The swab of claim 28, wherein the one-piece swab is made of one of: bamboo and wood.
